SEQUENCE LISTING (247 sequences)

SEQ ID NO: 1 - protein, 9 aa, Artificial Sequence
  Synthetic sequence derived from erythropoietin
  MISC_FEATURE: Synthetic sequence derived from erythropoietin
  MISC_FEATURE (1)..(1): X = phe, met ,tyr, leu, ile, val, or trp
  MISC_FEATURE (6)..(6): X = val, ser, thr, cys, pro, ala, leu, ile, val or phe
  MISC_FEATURE (7)..(7): X = met, his, or arg
  MISC_FEATURE (8)..(8): X = unknown
  MISC_FEATURE (9)..(9): X = met, his, trp, asp, or glu
XMTWIXXXX

SEQ ID NO: 2 - protein, 9 aa, Artificial Sequence
  Synthetic sequence derived from erythropoietin
  MISC_FEATURE: Synthetic sequence derived from erythropoietin
  MISC_FEATURE (1)..(1): X = phe, met, or tyr
  MISC_FEATURE (4)..(4): X = unknown
  MISC_FEATURE (6)..(6): X = val, ser, or thr
  MISC_FEATURE (7)..(7): X = met or his
  MISC_FEATURE (8)..(8): X = unknown
  MISC_FEATURE (9)..(9): X = met or his
XMTXIXXXX

SEQ ID NO: 3 - protein, 193 aa, Homo sapiens
  MISC_FEATURE: Erythropoietin precursor, NCBI Entrez Protein Database Accession number P01588
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 4 - protein, 136 aa, Oncorhynchus keta
  MISC_FEATURE: Calcitonin 1 precursor,  NCBI Entrez Protein Database Accession No. P01263
MVMMKLSALLIAYFLVICQMYSSHAAPARTGLESMTDQVTLTDYEARRLLNAIVKEFVQMTSEELEQQANEGNSLDRPMSKRCSNLSTCVLGKLSQELHKLQTYPRTNTGSGTPGKKRSLPESNRYASYGDSYDGI

SEQ ID NO: 5 - protein, 217 aa, Homo sapiens
  MISC_FEATURE: Somatotropin precursor, NCBI Entrez Protein Database Accession No. P01241
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 6 - protein, 110 aa, Homo sapiens
  MISC_FEATURE: Insulin precursor, NCBI Entrez Protein Database Accession No. P01308
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 7 - protein, 110 aa, Homo sapiens; identical to SEQ ID NO: 6
  MISC_FEATURE: Insulin Precursor, NCBI Entrez Protein Database Accession No. P01308

SEQ ID NO: 8 - protein, 187 aa, Homo sapiens [sequence omitted]

SEQ ID NO: 9 - protein, 193 aa, Homo sapiens; identical to SEQ ID NO: 3
  MISC_FEATURE: Amino Acid Sequence of Wild Type Human Erythropoietin (Fig. 10A)
  MISC_FEATURE (1)..(27): Signal sequence amino acids
  MISC_FEATURE (28)..(193): EPO protein amino acids

SEQ ID NO: 10 - protein, 193 aa, Artificial Sequence
  Synthetic sequence derived from erythropoietin
  MISC_FEATURE: Amino Acid Sequence of Modified Human Erythropoietin Construct 1 (Fig. 10B)
  MISC_FEATURE (1)..(27): Signal sequence amino acid
  MISC_FEATURE (28)..(193): EPO protein amino acids
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGPRSLTTLLRALGAQKEAISPPDAASAAPLRTITADTFRKLFRVYDNFLRGKLKLYTGEACRTGDR

SEQ ID NO: 11 - protein, 193 aa, Artificial Sequence
  Synthetic sequence derived from erythropoietin
  MISC_FEATURE: Amino Acid Sequence of Modified Human Erythropoietin Construct 2 (Fig. 10C)
  MISC_FEATURE (1)..(27): Signal sequence amino acids
  MISC_FEATURE (28)..(193): EPO protein amino acids
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTDLLRALGAQKEAISPPDAASAAPLRTITADTFRKLFRVYDNFLRGKLKLYTGEACRTGDR

SEQ ID NO: 12 - protein, 193 aa, Artificial Sequence
  Synthetic sequence derived from erythropoietin
  MISC_FEATURE: Amino Acid Sequence of Modified Human Erythropoietin Construct 3 (Fig. 10D)
  MISC_FEATURE (1)..(27): Signal sequence amino acids
  MISC_FEATURE (28)..(193): EPO protein amino acids
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGGRSLTDLLRALGAQKEAISPPDAASAAPLRTITADTFRKLFRVYDNFLRGKLKLYTGEACRTGDR

SEQ ID NO: 13 - protein, 193 aa, Artificial Sequence
  Synthetic sequence derived from erythropoietin
  MISC_FEATURE: Amino Acid Sequence of Modified Human Erythropoietin Construct 4 (Fig. 10E)
  MISC_FEATURE (1)..(27): Signal sequence amino acids
  MISC_FEATURE (28)..(193): EPO protein amino acids
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGPRSLTDLLRALGAQKEAISPPDAASAAPLRTITADTFRKLFRVYDNFLRGKLKLYTGEACRTGDR

SEQ ID NO: 14 - protein, 193 aa, Artificial Sequence
  Synthetic sequence derived from erythropoietin
  MISC_FEATURE: Amino Acid Sequence of Modified Human Erythropoietin Construct 5 (Fig. 10F)
  MISC_FEATURE (1)..(27): Signal sequence amino acids
  MISC_FEATURE (28)..(193): EPO protein amino acids
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGSRSLTDLLRALGAQKEAISPPDAASAAPLRTITADTFRKLFRVYDNFLRGKLKLYTGEACRTGDR

SEQ ID NO: 15 - protein, 14 aa, Artificial Sequence
  Synthetic sequence derived from erythropoietin
  MISC_FEATURE: Synthetic Sequence derived from erythropoietin
QYIKANSKFIGITE

SEQ ID NO: 16 - protein, 13 aa, Artificial Sequence
  Synthetic sequence derived from erythropoietin
  MISC_FEATURE: Synthetic sequence derived from erythropoietin
PKYVKQNTLKLAT

SEQ ID NO: 17 - protein, 20 aa, Artificial Sequence
  Synthetic sequence derived from erythropoietin
  MISC_FEATURE: Synthetic sequence derived from erythropoietin
AAYAAQGYKVLVLNPSVAAT

SEQ ID NO: 18 - protein, 15 aa, Artificial Sequence
  Synthetic sequence derived from erythropoietin
  MISC_FEATURE: Synthetic sequence derived from erythropoietin
LQSLTNLLSSNLSWL

SEQ ID NO: 19 - protein, 15 aa, Artificial Sequence
  Synthetic sequence derived from erythropoietin
  MISC_FEATURE: Synthetic sequence derived from erythropoietin
RHNWVNHAVPLAMKL

SEQ ID NO: 20 - protein, 15 aa, Artificial Sequence
  Synthetic sequence derived from erythropoietin
  MISC_FEATURE: Synthetic sequence derived from erythropoietin
APPRLICDSRVLERY

SEQ ID NO: 21 - protein, 15 aa, Artificial Sequence
  Synthetic sequence derived from erythropoietin
  MISC_FEATURE: Synthetic sequence derived from erythropoietin
ICDSRVLERYLLEAK

SEQ ID NO: 22 - protein, 15 aa, Artificial Sequence
  Synthetic sequence derived from erythropoietin
  MISC_FEATURE: Synthetic sequence derived from erythropoietin
VLERYLLEAKEAENI

SEQ ID NO: 23 - protein, 15 aa, Artificial Sequence
  Synthetic sequence derived from erythropoietin
  MISC_FEATURE: Synthetic sequence derived from erythropoietin
LLEAKEAENITTGCA

SEQ ID NO: 24 - protein, 15 aa, Artificial Sequence
  Synthetic sequence derived from erythropoietin
  MISC_FEATURE: Synthetic sequence derived from erythropoietin
EAENITTGCAEHCSL

SEQ ID NO: 25 - protein, 15 aa, Artificial Sequence
  Synthetic sequence derived from erythropoietin
  MISC_FEATURE: Synthetic sequence derived from erythropoietin
TTGCAEHCSLNENIT

SEQ ID NO: 26 - protein, 15 aa, Artificial Sequence
  Synthetic sequence derived from erythropoietin
  MISC_FEATURE: Synthetic sequence derived from erythropoietin
EHCSLNENITVPDTK

SEQ ID NO: 27 - protein, 15 aa, Artificial Sequence
  Synthetic sequence derived from erythropoietin
  MISC_FEATURE: Synthetic sequence derived from erythropoietin
NENITVPDTKVNFYA

SEQ ID NO: 28 - protein, 15 aa, Artificial Sequence
  Synthetic sequence derived from erythropoietin
  MISC_FEATURE: Synthetic sequence derived from erythropoietin
VPDTKVNFYAWKRME

SEQ ID NO: 29 - protein, 15 aa, Artificial Sequence
  Synthetic sequence derived from erythropoietin
  MISC_FEATURE: Synthetic sequence derived from erythropoietin
VNFYAWKRMEVGQQA

SEQ ID NO: 30 - protein, 15 aa, Artificial Sequence
  Synthetic sequence derived from erythropoietin
  MISC_FEATURE: Synthetic sequence derived from erythropoietin
WKRMEVGQQAVEVWQ

SEQ ID NO: 31 - protein, 15 aa, Artificial Sequence
  Synthetic sequence derived from erythropoietin
  MISC_FEATURE: Synthetic sequence derived from erythropoietin
VGQQAVEVWQGLALL

SEQ ID NO: 32 - protein, 15 aa, Artificial Sequence
  Synthetic sequence derived from erythropoietin
  MISC_FEATURE: Synthetic sequence derived from erythropoietin
VEVWQGLALLSEAVL

SEQ ID NO: 33 - protein, 15 aa, Artificial Sequence
  Synthetic sequence derived from erythropoietin
  MISC_FEATURE: Synthetic sequence derived from erythropoietin
GLALLSEAVLRGQAL

SEQ ID NO: 34 - protein, 15 aa, Artificial Sequence
  Synthetic sequence derived from erythropoietin
  MISC_FEATURE: Synthetic sequence derived from erythropoietin
SEAVLRGQALLVNSS

SEQ ID NO: 35 - protein, 15 aa, Artificial Sequence
  Synthetic sequence derived from erythropoietin
  MISC_FEATURE: Synthetic sequence derived from erythropoietin
RGQALLVNSSQPWEP

SEQ ID NO: 36 - protein, 15 aa, Artificial Sequence
  Synthetic sequence derived from erythropoietin
  MISC_FEATURE: Synthetic sequence derived from erythropoietin
LVNSSQPWEPLQLHV

SEQ ID NO: 37 - protein, 15 aa, Artificial Sequence
  Synthetic sequence derived from erythropoietin
  MISC_FEATURE: Synthetic sequence derived from erythropoietin
QPWEPLQLHVDKAVS

SEQ ID NO: 38 - protein, 15 aa, Artificial Sequence
  Synthetic sequence derived from erythropoietin
  MISC_FEATURE: Synthetic sequence derived from erythropoietin
LQLHVDKAVSGLRSL

SEQ ID NO: 39 - protein, 15 aa, Artificial Sequence
  Synthetic sequence derived from erythropoietin
  MISC_FEATURE: Synthetic sequence derived from erythropoietin
DKAVSGLRSLTTLLR

SEQ ID NO: 40 - protein, 15 aa, Artificial Sequence
  Synthetic sequence derived from erythropoietin
  MISC_FEATURE: Synthetic sequence derived from erythropoietin
GLRSLTTLLRALGAQ

SEQ ID NO: 41 - protein, 15 aa, Artificial Sequence
  Synthetic sequence derived from erythropoietin
  MISC_FEATURE: Synthetic sequence derived from erythropoietin
TTLLRALGAQKEAIS

SEQ ID NO: 42 - protein, 15 aa, Artificial Sequence
  Synthetic sequence derived from erythropoietin
  MISC_FEATURE: Synthetic sequence derived from erythropoietin
ALGAQKEAISPPDAA

SEQ ID NO: 43 - protein, 15 aa, Artificial Sequence
  Synthetic sequence derived from erythropoietin
  MISC_FEATURE: Synthetic sequence derived from erythropoietin
KEAISPPDAASAAPL

SEQ ID NO: 44 - protein, 15 aa, Artificial Sequence
  Synthetic sequence derived from erythropoietin
  MISC_FEATURE: Synthetic sequence derived from erythropoietin
PPDAASAAPLRTITA

SEQ ID NO: 45 - protein, 15 aa, Artificial Sequence
  Synthetic sequence derived from erythropoietin
  MISC_FEATURE: Synthetic sequence derived from erythropoietin
SAAPLRTITADTFRK

SEQ ID NO: 46 - protein, 15 aa, Artificial Sequence
  Synthetic sequence derived from erythropoietin
  MISC_FEATURE: Synthetic sequence derived from erythropoietin
RTITADTFRKLFRVY

SEQ ID NO: 47 - protein, 15 aa, Artificial Sequence
  Synthetic sequence derived from erythropoietin
  MISC_FEATURE: Synthetic sequence derived from erythropoietin
DTFRKLFRVYSNFLR

SEQ ID NO: 48 - protein, 15 aa, Artificial Sequence
  Synthetic sequence derived from erythropoietin
  MISC_FEATURE: Synthetic sequence derived from erythropoietin
LFRVYSNFLRGKLKL

SEQ ID NO: 49 - protein, 15 aa, Artificial Sequence
  Synthetic sequence derived from erythropoietin
  MISC_FEATURE: Synthetic sequence derived from erythropoietin
SNFLRGKLKLYTGEA

SEQ ID NO: 50 - protein, 15 aa, Artificial Sequence
  Synthetic sequence derived from erythropoietin
  MISC_FEATURE: Synthetic sequence derived from erythropoietin
KLKLYTGEACRTGDR

SEQ ID NO: 51 - protein, 15 aa, Artificial Sequence
  Synthetic sequence derived from erythropoietin
  MISC_FEATURE: Synthetic sequence derived from erythropoietin
CSNLSTCVLGKLSQE

SEQ ID NO: 52 - protein, 15 aa, Artificial Sequence
  Synthetic sequence derived from erythropoietin
  MISC_FEATURE: Synthetic sequence derived from erythropoietin
TCVLGKLSQELHKLQ

SEQ ID NO: 53 - protein, 15 aa, Artificial Sequence
  Synthetic sequence derived from erythropoietin
  MISC_FEATURE: Synthetic sequence derived from erythropoietin
KLSQELHKLQTYPRT

SEQ ID NO: 54 - protein, 15 aa, Artificial Sequence
  Synthetic sequence derived from erythropoietin
  MISC_FEATURE: Synthetic sequence derived from erythropoietin
LHKLQTYPRTNTGSG

SEQ ID NO: 55 - protein, 15 aa, Artificial Sequence
  Synthetic sequence derived from erythropoietin
  MISC_FEATURE: Synthetic sequence derived from erythropoietin
KLQTYPRTNTGSGTP

SEQ ID NO: 56 - protein, 12 aa, Artificial Sequence
  Synthetic sequence derived from erythropoietin
  MISC_FEATURE: Synthetic sequence derived from erythropoietin
TYPRTNTGSGTP

SEQ ID NO: 57 - protein, 15 aa, Artificial Sequence
  Synthetic sequence derived from erythropoietin
  MISC_FEATURE: Synthetic sequence derived from erythropoietin
FPTIPLSRLFDNASL

SEQ ID NO: 58 - protein, 15 aa, Artificial Sequence
  Synthetic sequence derived from erythropoietin
  MISC_FEATURE: Synthetic sequence derived from erythropoietin
RLFDNASLRAHRLHQ

SEQ ID NO: 59 - protein, 15 aa, Artificial Sequence
  Synthetic sequence derived from erythropoietin
  MISC_FEATURE: Synthetic sequence derived from erythropoietin
LRAHRLHQLAFDTYQ

SEQ ID NO: 60 - protein, 15 aa, Artificial Sequence
  Synthetic sequence derived from erythropoietin
  MISC_FEATURE: Synthetic sequence derived from erythropoietin
QLAFDTYQEFEEAYI

SEQ ID NO: 61 - protein, 15 aa, Artificial Sequence
  Synthetic sequence derived from erythropoietin
  MISC_FEATURE: Synthetic sequence derived from erythropoietin
QEFEEAYIPKEQKYS

SEQ ID NO: 62 - protein, 15 aa, Artificial Sequence
  Synthetic sequence derived from erythropoietin
  MISC_FEATURE: Synthetic sequence derived from erythropoietin
IPKEQKYSFLQNPQT

SEQ ID NO: 63 - protein, 15 aa, Artificial Sequence
  Synthetic sequence derived from erythropoietin
  MISC_FEATURE: Synthetic sequence derived from erythropoietin
SFLQNPQTSLCFSES

SEQ ID NO: 64 - protein, 15 aa, Artificial Sequence
  Synthetic sequence derived from erythropoietin
  MISC_FEATURE: Synthetic sequence derived from erythropoietin
TSLCFSESIPTPSNR

SEQ ID NO: 65 - protein, 15 aa, Artificial Sequence
  Synthetic sequence derived from erythropoietin
  MISC_FEATURE: Synthetic sequence derived from erythropoietin
SIPTPSNREETQQKS

SEQ ID NO: 66 - protein, 15 aa, Artificial Sequence
  Synthetic sequence derived from erythropoietin
  MISC_FEATURE: Synthetic sequence derived from erythropoietin
REETQQKSNLELLRI

SEQ ID NO: 67 - protein, 15 aa, Artificial Sequence
  Synthetic sequence derived from erythropoietin
  MISC_FEATURE: Synthetic sequence derived from erythropoietin
SNLELLRISLLLIQS

SEQ ID NO: 68 - protein, 15 aa, Artificial Sequence
  Synthetic sequence derived from erythropoietin
  MISC_FEATURE: Synthetic sequence derived from erythropoietin
ISLLLIQSWLEPVQF

SEQ ID NO: 69 - protein, 15 aa, Artificial Sequence
  Synthetic sequence derived from erythropoietin
  MISC_FEATURE: Synthetic sequence derived from erythropoietin
SWLEPVQFLRSVFAN

SEQ ID NO: 70 - protein, 15 aa, Artificial Sequence
  Synthetic sequence derived from erythropoietin
  MISC_FEATURE: Synthetic sequence derived from erythropoietin
FLRSVFANSLVYGAS

SEQ ID NO: 71 - protein, 15 aa, Artificial Sequence
  Synthetic sequence derived from erythropoietin
  MISC_FEATURE: Synthetic sequence derived from erythropoietin
NSLVYGASDSNVYDL

SEQ ID NO: 72 - protein, 15 aa, Artificial Sequence
  Synthetic sequence derived from erythropoietin
  MISC_FEATURE: Synthetic sequence derived from erythropoietin
SDSNVYDLLKDLEEG

SEQ ID NO: 73 - protein, 15 aa, Artificial Sequence
  Synthetic sequence derived from erythropoietin
  MISC_FEATURE: Synthetic sequence derived from erythropoietin
LLKDLEEGIQTLMGR

SEQ ID NO: 74 - protein, 15 aa, Artificial Sequence
  Synthetic sequence derived from erythropoietin
  MISC_FEATURE: Synthetic sequence derived from erythropoietin
GIQTLMGRLEDGSPR

SEQ ID NO: 75 - protein, 15 aa, Artificial Sequence
  Synthetic sequence derived from erythropoietin
  MISC_FEATURE: Synthetic sequence derived from erythropoietin
RLEDGSPRTGQIFKQ

SEQ ID NO: 76 - protein, 15 aa, Artificial Sequence
  Synthetic sequence derived from erythropoietin
  MISC_FEATURE: Synthetic sequence derived from erythropoietin
RTGQIFKQTYSKFDT

SEQ ID NO: 77 - protein, 15 aa, Artificial Sequence
  Synthetic sequence derived from erythropoietin
  MISC_FEATURE: Synthetic sequence derived from erythropoietin
QTYSKFDTNSHNDDA

SEQ ID NO: 78 - protein, 15 aa, Artificial Sequence
  Synthetic sequence derived from erythropoietin
  MISC_FEATURE: Synthetic sequence derived from erythropoietin
TNSHNDDALLKNYGL

SEQ ID NO: 79 - protein, 15 aa, Artificial Sequence
  Synthetic sequence derived from erythropoietin
  MISC_FEATURE: Synthetic sequence derived from erythropoietin
ALLKNYGLLYCFRKD

SEQ ID NO: 80 - protein, 15 aa, Artificial Sequence
  Synthetic sequence derived from erythropoietin
  MISC_FEATURE: Synthetic sequence derived from erythropoietin
LLYCFRKDMDKVETF

SEQ ID NO: 81 - protein, 15 aa, Artificial Sequence
  Synthetic sequence derived from erythropoietin
  MISC_FEATURE: Synthetic sequence derived from erythropoietin
DMDKVETFLRIVQCR

SEQ ID NO: 82 - protein, 16 aa, Artificial Sequence
  Synthetic sequence derived from erythropoietin
  MISC_FEATURE: Synthetic sequence derived from erythropoietin
FLRIVQCRSVEGSCGF

SEQ ID NO: 83 - protein, 15 aa, Artificial Sequence
  Synthetic sequence derived from erythropoietin
  MISC_FEATURE: Synthetic sequence derived from erythropoietin
MSYNLLGFLQRSSNC

SEQ ID NO: 84 - protein, 15 aa, Artificial Sequence [sequence omitted]

SEQ ID NO: 85 - protein, 15 aa, Artificial Sequence
  Synthetic sequence derived from erythropoietin
  MISC_FEATURE: Synthetic sequence derived from erythropoietin
RSSNCQCQKLLWQLN

SEQ ID NO: 86 - protein, 15 aa, Artificial Sequence
  Synthetic sequence derived from erythropoietin
  MISC_FEATURE: Synthetic sequence derived from erythropoietin
QCQKLLWQLNGRLEY

SEQ ID NO: 87 - protein, 15 aa, Artificial Sequence
  Synthetic sequence derived from erythropoietin
  MISC_FEATURE: Synthetic sequence derived from erythropoietin
LWQLNGRLEYCLKDR

SEQ ID NO: 88 - protein, 15 aa, Artificial Sequence
  Synthetic sequence derived from erythropoietin
  MISC_FEATURE: Synthetic sequence derived from erythropoietin
GRLEYCLKDRRNFDI

SEQ ID NO: 89 - protein, 15 aa, Artificial Sequence
  Synthetic sequence derived from erythropoietin
  MISC_FEATURE: Synthetic sequence derived from erythropoietin
CLKDRRNFDIPEEIK

SEQ ID NO: 90 - protein, 15 aa, Artificial Sequence
  Synthetic sequence derived from erythropoietin
  MISC_FEATURE: Synthetic sequence derived from erythropoietin
RNFDIPEEIKQLQQF

SEQ ID NO: 91 - protein, 15 aa, Artificial Sequence
  Synthetic sequence derived from erythropoietin
  MISC_FEATURE: Synthetic sequence derived from erythropoietin
PEEIKQLQQFQKEDA

SEQ ID NO: 92 - protein, 15 aa, Artificial Sequence [sequence omitted]

SEQ ID NO: 93 - protein, 15 aa, Artificial Sequence
  Synthetic sequence derived from erythropoietin
  MISC_FEATURE: Synthetic sequence derived from erythropoietin
QKEDAAVTIYEMLQN

SEQ ID NO: 94 - protein, 15 aa, Artificial Sequence
  Synthetic sequence derived from erythropoietin
  MISC_FEATURE: Synthetic sequence derived from erythropoietin
AVTIYEMLQNIFAIF

SEQ ID NO: 95 - protein, 15 aa, Artificial Sequence
  Synthetic sequence derived from erythropoietin
  MISC_FEATURE: Synthetic sequence derived from erythropoietin
EMLQNIFAIFRQDSS

SEQ ID NO: 96 - protein, 15 aa, Artificial Sequence
  Synthetic sequence derived from erythropoietin
  MISC_FEATURE: Synthetic sequence derived from erythropoietin
IFAIFRQDSSSTGWN

SEQ ID NO: 97 - protein, 15 aa, Artificial Sequence
  Synthetic sequence derived from erythropoietin
  MISC_FEATURE: Synthetic sequence derived from erythropoietin
RQDSSSTGWNETIVE

SEQ ID NO: 98 - protein, 15 aa, Artificial Sequence
  Synthetic sequence derived from erythropoietin
  MISC_FEATURE: Synthetic sequence derived from erythropoietin
STGWNETIVENLLAN

SEQ ID NO: 99 - protein, 15 aa, Artificial Sequence
  Synthetic sequence derived from erythropoietin
  MISC_FEATURE: Synthetic sequence derived from erythropoietin
ETIVENLLANVYHQR

SEQ ID NO: 100 - protein, 15 aa, Artificial Sequence
  Synthetic sequence derived from erythropoietin
  MISC_FEATURE: Synthetic sequence derived from erythropoietin
NLLANVYHQRNHLKT

SEQ ID NO: 101 - protein, 15 aa, Artificial Sequence
  Synthetic sequence derived from erythropoietin
  MISC_FEATURE: Synthetic sequence derived from erythropoietin
VYHQRNHLKTVLEEK

SEQ ID NO: 102 - protein, 15 aa, Artificial Sequence
  Synthetic sequence derived from erythropoietin
  MISC_FEATURE: Synthetic sequence derived from erythropoietin
NHLKTVLEEKLEKED

SEQ ID NO: 103 - protein, 15 aa, Artificial Sequence
  Synthetic sequence derived from erythropoietin
  MISC_FEATURE: Synthetic sequence derived from erythropoietin
VLEEKLEKEDFTRGK

SEQ ID NO: 104 - protein, 15 aa, Artificial Sequence [sequence omitted]

SEQ ID NO: 105 - protein, 15 aa, Artificial Sequence
  Synthetic sequence derived from erythropoietin
  MISC_FEATURE: Synthetic sequence derived from erythropoietin
FTRGKRMSSLHLKRY

SEQ ID NO: 106 - protein, 15 aa, Artificial Sequence
  Synthetic sequence derived from erythropoietin
  MISC_FEATURE: Synthetic sequence derived from erythropoietin
RMSSLHLKRYYGRIL

SEQ ID NO: 107 - protein, 15 aa, Artificial Sequence
  Synthetic sequence derived from erythropoietin
  MISC_FEATURE: Synthetic sequence derived from erythropoietin
HLKRYYGRILHYLKA

SEQ ID NO: 108 - protein, 15 aa, Artificial Sequence
  Synthetic sequence derived from erythropoietin
  MISC_FEATURE: Synthetic sequence derived from erythropoietin
YGRILHYLKAKEDSH

SEQ ID NO: 109 - protein, 15 aa, Artificial Sequence
  Synthetic sequence derived from erythropoietin
  MISC_FEATURE: Synthetic sequence derived from erythropoietin
HYLKAKEDSHCAWTI

SEQ ID NO: 110 - protein, 15 aa, Artificial Sequence
  Synthetic sequence derived from erythropoietin
  MISC_FEATURE: Synthetic sequence derived from erythropoietin
KEDSHCAWTIVRVEI

SEQ ID NO: 111 - protein, 15 aa, Artificial Sequence
  Synthetic sequence derived from erythropoietin
  MISC_FEATURE: Synthetic sequence derived from erythropoietin
CAWTIVRVEILRNFY

SEQ ID NO: 112 - protein, 15 aa, Artificial Sequence
  Synthetic sequence derived from erythropoietin
  MISC_FEATURE: Synthetic sequence derived from erythropoietin
VRVEILRNFYVINRL

SEQ ID NO: 113 - protein, 15 aa, Artificial Sequence [sequence omitted]

SEQ ID NO: 114 - protein, 15 aa, Artificial Sequence
  Synthetic sequence derived from erythropoietin
  MISC_FEATURE: Synthetic sequence derived from erythropoietin
GIVEQCCTSICSLYQ

SEQ ID NO: 115 - protein, 15 aa, Artificial Sequence
  Synthetic sequence derived from erythropoietin
  MISC_FEATURE: Synthetic sequence derived from erythropoietin
EQCCTSICSLYQLEN

SEQ ID NO: 116 - protein, 14 aa, Artificial Sequence
  Synthetic sequence derived from erythropoietin
  MISC_FEATURE: Synthetic sequence derived from erythropoietin
TSICSLYQLENYCN

SEQ ID NO: 117 - protein, 15 aa, Artificial Sequence
  Synthetic sequence derived from erythropoietin
  MISC_FEATURE: Synthetic sequence derived from erythropoietin
FVNQHLCGSHLVEAL

SEQ ID NO: 118 - protein, 15 aa, Artificial Sequence
  Synthetic sequence derived from erythropoietin
  MISC_FEATURE: Synthetic sequence derived from erythropoietin
QHLCGSHLVEALYLV

SEQ ID NO: 119 - protein, 15 aa, Artificial Sequence
  Synthetic sequence derived from erythropoietin
  MISC_FEATURE: Synthetic sequence derived from erythropoietin
GSHLVEALYLVCGER

SEQ ID NO: 120 - protein, 15 aa, Artificial Sequence
  Synthetic sequence derived from erythropoietin
  MISC_FEATURE: Synthetic sequence derived from erythropoietin
VEALYLVCGERGFFY

SEQ ID NO: 121 - protein, 15 aa, Artificial Sequence
  Synthetic sequence derived from erythropoietin
  MISC_FEATURE: Synthetic sequence derived from erythropoietin
YLVCGERGFFYTPKT

SEQ ID NO: 122 - protein, 13 aa, Artificial Sequence
  Synthetic sequence derived from erythropoietin
  MISC_FEATURE: Synthetic sequence derived from erythropoietin
VCGERGFFYTPKT

SEQ ID NO: 123 - protein, 20 aa, Artificial Sequence
  Synthetic sequence derived from erythropoietin
  MISC_FEATURE: Synthetic sequence derived from erythropoietin
APYHFDLSGHAFGSMAKKGE

SEQ ID NO: 124 - protein, 16 aa, Artificial Sequence
  Synthetic sequence derived from erythropoietin
  MISC_FEATURE: Synthetic sequence derived from erythropoietin
AVLEDPYILLVSSKVS

SEQ ID NO: 125 - protein, 15 aa, Artificial Sequence
  Synthetic sequence derived from erythropoietin
  MISC_FEATURE: Synthetic sequence derived from erythropoietin
DALESIMTTKSVSFR

SEQ ID NO: 126 - protein, 21 aa, Artificial Sequence
  Synthetic sequence derived from erythropoietin
  MISC_FEATURE: Synthetic sequence derived from erythropoietin
DIEKKIAKMEKASSVFNVVNS

SEQ ID NO: 127 - protein, 12 aa, Artificial Sequence
  Synthetic sequence derived from erythropoietin
  MISC_FEATURE: Synthetic sequence derived from erythropoietin
DNVLDHLTGRSC

SEQ ID NO: 128 - protein, 18 aa, Artificial Sequence
  Synthetic sequence derived from erythropoietin
  MISC_FEATURE: Synthetic sequence derived from erythropoietin
DTPYLDITYHFVMQRLPL

SEQ ID NO: 129 - protein, 15 aa, Artificial Sequence
  Synthetic sequence derived from erythropoietin
  MISC_FEATURE: Synthetic sequence derived from erythropoietin
DYSYLQDSDPDSFQD

SEQ ID NO: 130 - protein, 13 aa, Artificial Sequence
  Synthetic sequence derived from erythropoietin
  MISC_FEATURE: Synthetic sequence derived from erythropoietin
EFVVEFDLPGIKA

SEQ ID NO: 131 - protein, 20 aa, Artificial Sequence
  Synthetic sequence derived from erythropoietin
  MISC_FEATURE: Synthetic sequence derived from erythropoietin
ESWGAVWRIDTPDKLTGPFT

SEQ ID NO: 132 - protein, 15 aa, Artificial Sequence
  Synthetic sequence derived from erythropoietin
  MISC_FEATURE: Synthetic sequence derived from erythropoietin
EVWREEAYHAADIKD

SEQ ID NO: 133 - protein, 23 aa, Artificial Sequence
  Synthetic sequence derived from erythropoietin
  MISC_FEATURE: Synthetic sequence derived from erythropoietin
FNNFTVSFWLRVPKVSASHLEQY

SEQ ID NO: 134 - protein, 21 aa, Artificial Sequence
  Synthetic sequence derived from erythropoietin
  MISC_FEATURE: Synthetic sequence derived from erythropoietin
GDVVAVVDIKEKGKDKWIELK

SEQ ID NO: 135 - protein, 14 aa, Artificial Sequence
  Synthetic sequence derived from erythropoietin
  MISC_FEATURE: Synthetic sequence derived from erythropoietin
GYKVLVLNPSVAAT

SEQ ID NO: 136 - protein, 24 aa, Artificial Sequence
  Synthetic sequence derived from erythropoietin
  MISC_FEATURE: Synthetic sequence derived from erythropoietin
IVHATGFKQSSKALQRPVASDFEP

SEQ ID NO: 137 - protein, 15 aa, Artificial Sequence
  Synthetic sequence derived from erythropoietin
  MISC_FEATURE: Synthetic sequence derived from erythropoietin
LLPLLEKVIGAGKPL

SEQ ID NO: 138 - protein, 13 aa, Artificial Sequence
  Synthetic sequence derived from erythropoietin
  MISC_FEATURE: Synthetic sequence derived from erythropoietin
NGQIGNDPNRDIL

SEQ ID NO: 139 - protein, 20 aa, Artificial Sequence
  Synthetic sequence derived from erythropoietin
  MISC_FEATURE: Synthetic sequence derived from erythropoietin
NPVVHFFKNIVTPRTPPPSQ

SEQ ID NO: 140 - protein, 20 aa, Artificial Sequence
  Synthetic sequence derived from erythropoietin
  MISC_FEATURE: Synthetic sequence derived from erythropoietin
PHHTALRQAILCWGELMTLA

SEQ ID NO: 141 - protein, 13 aa, Artificial Sequence
  Synthetic sequence derived from erythropoietin
  MISC_FEATURE: Synthetic sequence derived from erythropoietin
PKYVKQNTLKLAT

SEQ ID NO: 142 - protein, 27 aa, Artificial Sequence
  Synthetic sequence derived from erythropoietin
  MISC_FEATURE: Synthetic sequence derived from erythropoietin
PLGFFPDHQLDPAFGANSNNPDWDFNP

SEQ ID NO: 143 - protein, 15 aa, Artificial Sequence
  Synthetic sequence derived from erythropoietin
  MISC_FEATURE: Synthetic sequence derived from erythropoietin
QNILLSNAPLGPQFP

SEQ ID NO: 144 - protein, 14 aa, Artificial Sequence
  Synthetic sequence derived from erythropoietin
  MISC_FEATURE: Synthetic sequence derived from erythropoietin
QYIKANSKFIGITE

SEQ ID NO: 145 - protein, 20 aa, Artificial Sequence
  Synthetic sequence derived from erythropoietin
  MISC_FEATURE: Synthetic sequence derived from erythropoietin
RDTGILDSIGRFFGGDRGAP

SEQ ID NO: 146 - protein, 20 aa, Artificial Sequence
  Synthetic sequence derived from erythropoietin
  MISC_FEATURE: Synthetic sequence derived from erythropoietin
VDAQGTLSKIFKLGGRDSRS

SEQ ID NO: 147 - protein, 20 aa, Artificial Sequence
  Synthetic sequence derived from erythropoietin
  MISC_FEATURE: Synthetic sequence derived from erythropoietin
WTTCQSIAFPSKTSASIGSL

SEQ ID NO: 148 - protein, 12 aa, Artificial Sequence
  Synthetic sequence derived from erythropoietin
  MISC_FEATURE: Synthetic sequence derived from erythropoietin
YKTIAYDEEARR

SEQ ID NO: 149 - protein, 18 aa, Artificial Sequence
  Synthetic sequence derived from erythropoietin
  MISC_FEATURE: Synthetic sequence derived from erythropoietin
YLDPLIRGLLARPAKLQV

SEQ ID NO: 150 - protein, 16 aa, Artificial Sequence
  Synthetic sequence derived from erythropoietin
  MISC_FEATURE: Synthetic sequence derived from erythropoietin
YSGPLKAEIAQRLEDV

SEQ ID NO: 151 - protein, 25 aa, Artificial Sequence
  Synthetic sequence derived from erythropoietin
  MISC_FEATURE: Synthetic sequence derived from erythropoietin
YTLLQAAPALDKLKLTGDEATGANI

SEQ ID NO: 152 - protein, 15 aa, Artificial Sequence
  Synthetic sequence derived from erythropoietin
  MISC_FEATURE: Synthetic sequence derived from erythropoietin
GARSLTTLLRALGAQ

SEQ ID NO: 153 - protein, 15 aa, Artificial Sequence
  Synthetic sequence derived from erythropoietin
  MISC_FEATURE: Synthetic sequence derived from erythropoietin
GDRSLTTLLRALGAQ

SEQ ID NO: 154 - protein, 15 aa, Artificial Sequence
  Synthetic sequence derived from erythropoietin
  MISC_FEATURE: Synthetic sequence derived from erythropoietin
GERSLTTLLRALGAQ

SEQ ID NO: 155 - protein, 15 aa, Artificial Sequence
  Synthetic sequence derived from erythropoietin
  MISC_FEATURE: Synthetic sequence derived from erythropoietin
GGRSLTTLLRALGAQ

SEQ ID NO: 156 - protein, 15 aa, Artificial Sequence
  Synthetic sequence derived from erythropoietin
  MISC_FEATURE: Synthetic sequence derived from erythropoietin
GHRSLTTLLRALGAQ

SEQ ID NO: 157 - protein, 15 aa, Artificial Sequence
  Synthetic sequence derived from erythropoietin
  MISC_FEATURE: Synthetic sequence derived from erythropoietin
GKRSLTTLLRALGAQ

SEQ ID NO: 158 - protein, 15 aa, Artificial Sequence
  Synthetic sequence derived from erythropoietin
  MISC_FEATURE: Synthetic sequence derived from erythropoietin
GNRSLTTLLRALGAQ

SEQ ID NO: 159 - protein, 15 aa, Artificial Sequence
  Synthetic sequence derived from erythropoietin
  MISC_FEATURE: Synthetic sequence derived from erythropoietin
GPRSLTTLLRALGAQ

SEQ ID NO: 160 - protein, 15 aa, Artificial Sequence
  Synthetic sequence derived from erythropoietin
  MISC_FEATURE: Synthetic sequence derived from erythropoietin
GQRSLTTLLRALGAQ

SEQ ID NO: 161 - protein, 15 aa, Artificial Sequence
  Synthetic sequence derived from erythropoietin
  MISC_FEATURE: Synthetic sequence derived from erythropoietin
GRRSLTTLLRALGAQ

SEQ ID NO: 162 - protein, 15 aa, Artificial Sequence
  Synthetic sequence derived from erythropoietin
  MISC_FEATURE: Synthetic sequence derived from erythropoietin
GSRSLTTLLRALGAQ

SEQ ID NO: 163 - protein, 15 aa, Artificial Sequence
  Synthetic sequence derived from erythropoietin
  MISC_FEATURE: Synthetic sequence derived from erythropoietin
GTRSLTTLLRALGAQ

SEQ ID NO: 164 - protein, 15 aa, Artificial Sequence
  Synthetic sequence derived from erythropoietin
  MISC_FEATURE: Synthetic sequence derived from erythropoietin
GLRSLTTLARALGAQ

SEQ ID NO: 165 - protein, 15 aa, Artificial Sequence
  Synthetic sequence derived from erythropoietin
  MISC_FEATURE: Synthetic sequence derived from erythropoietin
GLRSLTTLDRALGAQ

SEQ ID NO: 166 - protein, 15 aa, Artificial Sequence
  Synthetic sequence derived from erythropoietin
  MISC_FEATURE: Synthetic sequence derived from erythropoietin
GLRSLTTLERALGAQ

SEQ ID NO: 167 - protein, 15 aa, Artificial Sequence
  Synthetic sequence derived from erythropoietin
  MISC_FEATURE: Synthetic sequence derived from erythropoietin
GLRSLTTLGRALGAQ

SEQ ID NO: 168 - protein, 15 aa, Artificial Sequence
  Synthetic sequence derived from erythropoietin
  MISC_FEATURE: Synthetic sequence derived from erythropoietin
GLRSLTTLHRALGAQ

SEQ ID NO: 169 - protein, 15 aa, Artificial Sequence
  Synthetic sequence derived from erythropoietin
  MISC_FEATURE: Synthetic sequence derived from erythropoietin
GLRSLTTLKRALGAQ

SEQ ID NO: 170 - protein, 15 aa, Artificial Sequence
  Synthetic sequence derived from erythropoietin
  MISC_FEATURE: Synthetic sequence derived from erythropoietin
GLRSLTTLNRALGAQ

SEQ ID NO: 171 - protein, 15 aa, Artificial Sequence
  Synthetic sequence derived from erythropoietin
  MISC_FEATURE: Synthetic sequence derived from erythropoietin
GLRSLTTLPRALGAQ

SEQ ID NO: 172 - protein, 15 aa, Artificial Sequence
  Synthetic sequence derived from erythropoietin
  MISC_FEATURE: Synthetic sequence derived from erythropoietin
GLRSLTTLQRALGAQ

SEQ ID NO: 173 - protein, 15 aa, Artificial Sequence
  Synthetic sequence derived from erythropoietin
  MISC_FEATURE: Synthetic sequence derived from erythropoietin
GLRSLTTLRRALGAQ

SEQ ID NO: 174 - protein, 15 aa, Artificial Sequence
  Synthetic sequence derived from erythropoietin
  MISC_FEATURE: Synthetic sequence derived from erythropoietin
GLRSLTTLSRALGAQ

SEQ ID NO: 175 - protein, 15 aa, Artificial Sequence
  Synthetic sequence derived from erythropoietin
  MISC_FEATURE: Synthetic sequence derived from erythropoietin
GLRSLTTLTRALGAQ

SEQ ID NO: 176 - protein, 15 aa, Artificial Sequence
  Synthetic sequence derived from erythropoietin
  MISC_FEATURE: Synthetic sequence derived from erythropoietin
GLDSLTTLLRALGAQ

SEQ ID NO: 177 - protein, 15 aa, Artificial Sequence
  Synthetic sequence derived from erythropoietin
  MISC_FEATURE: Synthetic sequence derived from erythropoietin
GLGSLTTLLRALGAQ

SEQ ID NO: 178 - protein, 15 aa, Artificial Sequence
  Synthetic sequence derived from erythropoietin
  MISC_FEATURE: Synthetic sequence derived from erythropoietin
GLRDLTTLLRALGAQ

SEQ ID NO: 179 - protein, 15 aa, Artificial Sequence
  Synthetic sequence derived from erythropoietin
  MISC_FEATURE: Synthetic sequence derived from erythropoietin
GLRGLTTLLRALGAQ

SEQ ID NO: 180 - protein, 15 aa, Artificial Sequence
  Synthetic sequence derived from erythropoietin
  MISC_FEATURE: Synthetic sequence derived from erythropoietin
GLRRLTTLLRALGAQ

SEQ ID NO: 181 - protein, 15 aa, Artificial Sequence
  Synthetic sequence derived from erythropoietin
  MISC_FEATURE: Synthetic sequence derived from erythropoietin
GLRSDTTLLRALGAQ

SEQ ID NO: 182 - protein, 15 aa, Artificial Sequence
  Synthetic sequence derived from erythropoietin
  MISC_FEATURE: Synthetic sequence derived from erythropoietin
GLRSGTTLLRALGAQ

SEQ ID NO: 183 - protein, 15 aa, Artificial Sequence
  Synthetic sequence derived from erythropoietin
  MISC_FEATURE: Synthetic sequence derived from erythropoietin
GLRSRTTLLRALGAQ

SEQ ID NO: 184 - protein, 15 aa, Artificial Sequence
  Synthetic sequence derived from erythropoietin
  MISC_FEATURE: Synthetic sequence derived from erythropoietin
GLRSLDTLLRALGAQ

SEQ ID NO: 185 - protein, 15 aa, Artificial Sequence
  Synthetic sequence derived from erythropoietin
  MISC_FEATURE: Synthetic sequence derived from erythropoietin
GLRSLGTLLRALGAQ

SEQ ID NO: 186 - protein, 15 aa, Artificial Sequence
  Synthetic sequence derived from erythropoietin
  MISC_FEATURE: Synthetic sequence derived from erythropoietin
GLRSLRTLLRALGAQ

SEQ ID NO: 187 - protein, 15 aa, Artificial Sequence
  Synthetic sequence derived from erythropoietin
  MISC_FEATURE: Synthetic sequence derived from erythropoietin
GLRSLTDLLRALGAQ

SEQ ID NO: 188 - protein, 15 aa, Artificial Sequence
  Synthetic sequence derived from erythropoietin
  MISC_FEATURE: Synthetic sequence derived from erythropoietin
GLRSLTGLLRALGAQ

SEQ ID NO: 189 - protein, 15 aa, Artificial Sequence
  Synthetic sequence derived from erythropoietin
  MISC_FEATURE: Synthetic sequence derived from erythropoietin
GLRSLTRLLRALGAQ

SEQ ID NO: 190 - protein, 15 aa, Artificial Sequence
  Synthetic sequence derived from erythropoietin
  MISC_FEATURE: Synthetic sequence derived from erythropoietin
GLRSLTTDLRALGAQ

SEQ ID NO: 191 - protein, 15 aa, Artificial Sequence
  Synthetic sequence derived from erythropoietin
  MISC_FEATURE: Synthetic sequence derived from erythropoietin
GLRSLTTGLRALGAQ

SEQ ID NO: 192 - protein, 15 aa, Artificial Sequence
  Synthetic sequence derived from erythropoietin
  MISC_FEATURE: Synthetic sequence derived from erythropoietin
GLRSLTTRLRALGAQ

SEQ ID NO: 193 - protein, 15 aa, Artificial Sequence
  Synthetic sequence derived from erythropoietin
  MISC_FEATURE: Synthetic sequence derived from erythropoietin
GLRSLTTLLDALGAQ

SEQ ID NO: 194 - protein, 15 aa, Artificial Sequence
  Synthetic sequence derived from erythropoietin
  MISC_FEATURE: Synthetic sequence derived from erythropoietin
GLRSLTTLLGALGAQ

SEQ ID NO: 195 - protein, 15 aa, Artificial Sequence
  Synthetic sequence derived from erythropoietin
  MISC_FEATURE: Synthetic sequence derived from erythropoietin
GLRSLTTLLRALGDQ

SEQ ID NO: 196 - protein, 15 aa, Artificial Sequence
  Synthetic sequence derived from erythropoietin
  MISC_FEATURE: Synthetic sequence derived from erythropoietin
GLRSLTTLLRALGGQ

SEQ ID NO: 197 - protein, 15 aa, Artificial Sequence
  Synthetic sequence derived from erythropoietin
  MISC_FEATURE: Synthetic sequence derived from erythropoietin
GLRSLTTLLRALGRQ

SEQ ID NO: 198 - protein, 15 aa, Artificial Sequence
  Synthetic sequence derived from erythropoietin
  MISC_FEATURE: Synthetic sequence derived from erythropoietin
DTFRKAFRVYSNFLR

SEQ ID NO: 199 - protein, 15 aa, Artificial Sequence
  Synthetic sequence derived from erythropoietin
  MISC_FEATURE: Synthetic sequence derived from erythropoietin
DTFRKDFRVYSNFLR

SEQ ID NO: 200 - protein, 15 aa, Artificial Sequence
  Synthetic sequence derived from erythropoietin
  MISC_FEATURE: Synthetic sequence derived from erythropoietin
DTFRKEFRVYSNFLR

SEQ ID NO: 201 - protein, 15 aa, Artificial Sequence
  Synthetic sequence derived from erythropoietin
  MISC_FEATURE: Synthetic sequence derived from erythropoietin
DTFRKGFRVYSNFLR

SEQ ID NO: 202 - protein, 15 aa, Artificial Sequence
  Synthetic sequence derived from erythropoietin
  MISC_FEATURE: Synthetic sequence derived from erythropoietin
DTFRKHFRVYSNFLR

SEQ ID NO: 203 - protein, 15 aa, Artificial Sequence
  Synthetic sequence derived from erythropoietin
  MISC_FEATURE: Synthetic sequence derived from erythropoietin
DTFRKKFRVYSNFLR

SEQ ID NO: 204 - protein, 15 aa, Artificial Sequence
  Synthetic sequence derived from erythropoietin
  MISC_FEATURE: Synthetic sequence derived from erythropoietin
DTFRKNFRVYSNFLR

SEQ ID NO: 205 - protein, 15 aa, Artificial Sequence
  Synthetic sequence derived from erythropoietin
  MISC_FEATURE: Synthetic sequence derived from erythropoietin
DTFRKPFRVYSNFLR

SEQ ID NO: 206 - protein, 15 aa, Artificial Sequence
  Synthetic sequence derived from erythropoietin
  MISC_FEATURE: Synthetic sequence derived from erythropoietin
DTFRKQFRVYSNFLR

SEQ ID NO: 207 - protein, 15 aa, Artificial Sequence
  Synthetic sequence derived from erythropoietin
  MISC_FEATURE: Synthetic sequence derived from erythropoietin
DTFRKRFRVYSNFLR

SEQ ID NO: 208 - protein, 15 aa, Artificial Sequence
  Synthetic sequence derived from erythropoietin
  MISC_FEATURE: Synthetic sequence derived from erythropoietin
DTFRKSFRVYSNFLR

SEQ ID NO: 209 - protein, 15 aa, Artificial Sequence
  Synthetic sequence derived from erythropoietin
  MISC_FEATURE: Synthetic sequence derived from erythropoietin
DTFRKTFRVYSNFLR

SEQ ID NO: 210 - protein, 15 aa, Artificial Sequence
  Synthetic sequence derived from erythropoietin
  MISC_FEATURE: Synthetic sequence derived from erythropoietin
DTARKLFRVYSNFLR

SEQ ID NO: 211 - protein, 15 aa, Artificial Sequence
  Synthetic sequence derived from erythropoietin
  MISC_FEATURE: Synthetic sequence derived from erythropoietin
DTDRKLFRVYSNFLR

SEQ ID NO: 212 - protein, 15 aa, Artificial Sequence
  Synthetic sequence derived from erythropoietin
  MISC_FEATURE: Synthetic sequence derived from erythropoietin
DTERKLFRVYSNFLR

SEQ ID NO: 213 - protein, 15 aa, Artificial Sequence
  Synthetic sequence derived from erythropoietin
  MISC_FEATURE: Synthetic sequence derived from erythropoietin
DTGRKLFRVYSNFLR

SEQ ID NO: 214 - protein, 15 aa, Artificial Sequence
  Synthetic sequence derived from erythropoietin
  MISC_FEATURE: Synthetic sequence derived from erythropoietin
DTHRKLFRVYSNFLR

SEQ ID NO: 215 - protein, 15 aa, Artificial Sequence
  Synthetic sequence derived from erythropoietin
  MISC_FEATURE: Synthetic sequence derived from erythropoietin
DTKRKLFRVYSNFLR

SEQ ID NO: 216 - protein, 15 aa, Artificial Sequence
  Synthetic sequence derived from erythropoietin
  MISC_FEATURE: Synthetic sequence derived from erythropoietin
DTNRKLFRVYSNFLR

SEQ ID NO: 217 - protein, 15 aa, Artificial Sequence
  Synthetic sequence derived from erythropoietin
  MISC_FEATURE: Synthetic sequence derived from erythropoietin
DTPRKLFRVYSNFLR

SEQ ID NO: 218 - protein, 15 aa, Artificial Sequence
  Synthetic sequence derived from erythropoietin
  MISC_FEATURE: Synthetic sequence derived from erythropoietin
DTQRKLFRVYSNFLR

SEQ ID NO: 219 - protein, 15 aa, Artificial Sequence
  Synthetic sequence derived from erythropoietin
  MISC_FEATURE: Synthetic sequence derived from erythropoietin
DTRRKLFRVYSNFLR

SEQ ID NO: 220 - protein, 15 aa, Artificial Sequence
  Synthetic sequence derived from erythropoietin
  MISC_FEATURE: Synthetic sequence derived from erythropoietin
DTSRKLFRVYSNFLR

SEQ ID NO: 221 - protein, 15 aa, Artificial Sequence
  Synthetic sequence derived from erythropoietin
  MISC_FEATURE: Synthetic sequence derived from erythropoietin
DTTRKLFRVYSNFLR

SEQ ID NO: 222 - protein, 15 aa, Artificial Sequence
  Synthetic sequence derived from erythropoietin
  MISC_FEATURE: Synthetic sequence derived from erythropoietin
DTFRKLDRVYSNFLR

SEQ ID NO: 223 - protein, 15 aa, Artificial Sequence
  Synthetic sequence derived from erythropoietin
  MISC_FEATURE: Synthetic sequence derived from erythropoietin
DTFRKLGRVYSNFLR

SEQ ID NO: 224 - protein, 15 aa, Artificial Sequence
  Synthetic sequence derived from erythropoietin
  MISC_FEATURE: Synthetic sequence derived from erythropoietin
DTFRKLRRVYSNFLR

SEQ ID NO: 225 - protein, 15 aa, Artificial Sequence
  Synthetic sequence derived from erythropoietin
  MISC_FEATURE: Synthetic sequence derived from erythropoietin
DTFRKLFDVYSNFLR

SEQ ID NO: 226 - protein, 15 aa, Artificial Sequence
  Synthetic sequence derived from erythropoietin
  MISC_FEATURE: Synthetic sequence derived from erythropoietin
DTFRKLFGVYSNFLR

SEQ ID NO: 227 - protein, 15 aa, Artificial Sequence
  Synthetic sequence derived from erythropoietin
  MISC_FEATURE: Synthetic sequence derived from erythropoietin
DTFRKLFRDYSNFLR

SEQ ID NO: 228 - protein, 15 aa, Artificial Sequence
  Synthetic sequence derived from erythropoietin
  MISC_FEATURE: Synthetic sequence derived from erythropoietin
DTFRKLFRGYSNFLR

SEQ ID NO: 229 - protein, 15 aa, Artificial Sequence
  Synthetic sequence derived from erythropoietin
  MISC_FEATURE: Synthetic sequence derived from erythropoietin
DTFRKLFRRYSNFLR

SEQ ID NO: 230 - protein, 15 aa, Artificial Sequence
  Synthetic sequence derived from erythropoietin
  MISC_FEATURE: Synthetic sequence derived from erythropoietin
DTFRKLFRVDSNFLR

SEQ ID NO: 231 - protein, 15 aa, Artificial Sequence
  Synthetic sequence derived from erythropoietin
  MISC_FEATURE: Synthetic sequence derived from erythropoietin
DTFRKLFRVGSNFLR

SEQ ID NO: 232 - protein, 15 aa, Artificial Sequence
  Synthetic sequence derived from erythropoietin
  MISC_FEATURE: Synthetic sequence derived from erythropoietin
DTFRKLFRVRSNFLR

SEQ ID NO: 233 - protein, 15 aa, Artificial Sequence
  Synthetic sequence derived from erythropoietin
  MISC_FEATURE: Synthetic sequence derived from erythropoietin
DTFRKLFRVYDNFLR

SEQ ID NO: 234 - protein, 15 aa, Artificial Sequence
  Synthetic sequence derived from erythropoietin
  MISC_FEATURE: Synthetic sequence derived from erythropoietin
DTFRKLFRVYGNFLR

SEQ ID NO: 235 - protein, 15 aa, Artificial Sequence
  Synthetic sequence derived from erythropoietin
  MISC_FEATURE: Synthetic sequence derived from erythropoietin
DTFRKLFRVYRNFLR

SEQ ID NO: 236 - protein, 15 aa, Artificial Sequence
  Synthetic sequence derived from erythropoietin
  MISC_FEATURE: Synthetic sequence derived from erythropoietin
DTFRKLFRVYSDFLR

SEQ ID NO: 237 - protein, 15 aa, Artificial Sequence
  Synthetic sequence derived from erythropoietin
  MISC_FEATURE: Synthetic sequence derived from erythropoietin
DTFRKLFRVYSGFLR

SEQ ID NO: 238 - protein, 15 aa, Artificial Sequence
  Synthetic sequence derived from erythropoietin
  MISC_FEATURE: Synthetic sequence derived from erythropoietin
DTFRKLFRVYSRFLR

SEQ ID NO: 239 - protein, 15 aa, Artificial Sequence
  Synthetic sequence derived from erythropoietin
  MISC_FEATURE: Synthetic sequence derived from erythropoietin
DTFRKLFRVYSNDLR

SEQ ID NO: 240 - protein, 15 aa, Artificial Sequence
  Synthetic sequence derived from erythropoietin
  MISC_FEATURE: Synthetic sequence derived from erythropoietin
DTFRKLFRVYSNGLR

SEQ ID NO: 241 - protein, 15 aa, Artificial Sequence
  Synthetic sequence derived from erythropoietin
  MISC_FEATURE: Synthetic sequence derived from erythropoietin
DTFRKLFRVYSNRLR

SEQ ID NO: 242 - protein, 15 aa, Artificial Sequence
  Synthetic sequence derived from erythropoietin
  MISC_FEATURE: Synthetic sequence derived from erythropoietin
DTFRKLFRVYSNFDR

SEQ ID NO: 243 - protein, 15 aa, Artificial Sequence
  Synthetic sequence derived from erythropoietin
  MISC_FEATURE: Synthetic sequence derived from erythropoietin
DTFRKLFRVYSNFGR

SEQ ID NO: 244 - protein, 15 aa, Artificial Sequence
  Synthetic sequence derived from erythropoietin
  MISC_FEATURE: Synthetic sequence derived from erythropoietin
DTFRKLFRVYSNFRR

SEQ ID NO: 245 - protein, 15 aa, Artificial Sequence
  Synthetic sequence derived from erythropoietin
  MISC_FEATURE: Synthetic sequence derived from erythropoietin
GGRSLTDLLRALGAQ

SEQ ID NO: 246 - protein, 15 aa, Artificial Sequence
  Synthetic sequence derived from erythropoietin
  MISC_FEATURE: Synthetic sequence derived from erythropoietin
GSRSLTDLLRALGAQ

SEQ ID NO: 247 - protein, 15 aa, Artificial Sequence
  Synthetic sequence derived from erythropoietin
  MISC_FEATURE: Synthetic sequence derived from erythropoietin
DTFRKDFRVYDNFLR

(19) United States
(12) Patent Application Publication
(10) Pub. No.: US 2007/0184519 A1
(43) Pub. Date: Aug. 9, 2007

(54) Peptides, polypeptides, and proteins of reduced immunogenicity and methods for their production

(76) Inventors: Shabnam Tangri, San Diego, CA (US); Bianca Mothe, Oceanside, CA (US); Alessandro Sette, La Jolla, CA (US); Scott Southwood, Santee, CA (US); Kristen Briggs, Del Mar, CA (US); Robert W. Chesnut, Cardiff-by-the-Sea, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/550,675

(22) PCT Filed: Apr. 2, 2004
(86) PCT No.: PCT/US04/10353

§ 371 Date: May 22, 2006

Related U.S. Application Data
(60) Provisional application No. 60/459,939, filed on Apr. 2, 2003.

(51) Int. Cl. A61K 38/00; C07H 21/04; C12P 21/06; A61K 38/18; C07K 14/505

(52) U.S. Cl. 435/69.1; 435/320.1; 435/325; 530/399; 536/23.5; 514/12

(57) ABSTRACT

The subject invention provides peptides, polypeptides, proteins and/or antibodies of reduced immunogenicity. Also provided are methods of reducing the immunogenicity of peptides, polypeptides, proteins and/or antibodies. In certain embodiments, the immunogenicity of therapeutic peptides, polypeptides, proteins, and/or antibodies such as hormones, growth factors, and cytokines is reduced.

### CROSS-REFERENCE TO RELATED APPLICATIONS

This application claims benefit of priority under 35 U.S.C. §119(e)(1) to U.S. Provisional Patent Application No. 60/459,939, filed Apr. 2, 2003 (which is hereby incorporated by reference in its entirety). The present application also is a continuation-in-part of U.S. patent application Ser. No. 10/103,395, filed Mar. 20, 2002, which is a continuation-in-part of U.S. patent application Ser. No. 09/009,953, filed Jan. 21, 1998, now U.S. Pat. No. 6,413,517, issued Jul. 2, 2002, which claims benefit of priority under 35 U.S.C. § 119(e)(1) to U.S. Provisional Patent Application Nos. 60/036,713, filed Jan. 23, 1997 and 60/037,432 filed Feb. 7, 1997, each of which is incorporated herein by reference in its entirety, including all amino acid and/or polynucleotide sequences, sequence listings, figures, claims, and tables.

### BACKGROUND OF THE INVENTION

Helper T lymphocytes (HTL) play several important functions in immunity to pathogens. Firstly, they provide help for induction of both CTL and antibody responses. By both direct contact and by secreting cytokines such as IL2 and IL4, HTL promote and support the expansion and differentiation of T and B cell precursors into effector cells. In addition, HTL can also be effectors in their own right, an activity also mediated by direct cell contact and secretion of cytokines, such as IFNγ and TNFα. HTL have been shown to have direct effector activity in case of tumors, as well as viral, bacterial, parasitic, and fungal infections.

HTL recognize a complex formed between Class II MHC molecules and antigenic peptides, usually between 10 and 20 residues long, and with an average size of between 13 and 16 amino acids. Peptide-Class II interactions have been analyzed in detail, both at the structural and functional level, and peptide motifs specific for various human and mouse Class II molecules have been proposed.

Over the last few years, an ever-increasing number of therapeutic protein and antibody drugs have entered clinical trials or received approval for product registration. Several of these drugs are recombinant hormones, lymphokines and growth factors, such as insulin, factor VIII, interferons (IFN), Interleukin-2 (IL-2) and granulocyte macrophage colony stimulating factor (GM-CSF). Antibodies, such as the registered products Remicade (anti-TNF), Rituxan (anti-CD20) and Herceptin (anti-Her2neu) also comprise this category. In fact, 30-40% of all drug products currently in development are monoclonal antibodies targeting a wide variety of indications ranging from metabolic disorders to cancer and autoimmune diseases. Finally, as a result of the recent explosion in the volume of sequence and expression data provided by genomic/proteomic projects, new links between protein expression and pathological conditions are being discovered. These advances are predicted to further amplify the number of protein drug candidates developed to treat various pathological conditions.

Protein and antibody drugs are accessible to several different therapeutic applications. For example, in cases where pathology is caused by a protein deficiency (e.g., factor VIII in hemophiliacs) administration of a recombinant or purified protein product is often of therapeutic value. By contrast, pathologies associated with over-expression of a given protein may be treated by the administration of monoclonal antibodies directed against the overexpressed protein (e.g., use of Herceptin antibody targeted against HER 2/neu protein in breast cancer). In addition, synergistic to the increase in protein drugs and drug candidates is the development of exciting new technologies that allow the engineering of human proteins to achieve novel or dramatically improved pharmacological properties, for example, the development of Lispro, the fast-acting analog of insulin and the development of PROLEUKIN®, a single-substitution analog that is a non-aggregated and rapidly acting form of Interleukin-2.

Within this rapidly-growing category of therapeutics, a major and significant complication is frequently encountered, namely the development of immune reactions to the protein or antibody drug. The risk is especially high for chronically administered products. The route of delivery also plays an important role in rendering a protein or antibody drug immunogenic. Regardless of the cause, following activation of specific immune lymphocytes, a cascade of immune recognition events results in the formation of anti-drug antibodies. These antibodies, in turn, make the drugs less effective, ineffective, and/or cause other safety issues.

Substantial evidence indicates that immunogenicity issues exist with many of the currently marketed protein therapeutics. Well-documented examples include the development of antibodies against factor VIII in hemophiliacs treated with the drug (Bristol, et al. (2001) Hum. Gene. Ther, 12(13):1651; Kulkarni, et al. (2000) Am. J. Haematol, 67(4):240), against calcitonin in patients treated for osteoporosis (Grauer, A., et al. (1995) Exp. Clin Endocrinol., 103(6):345-351; Reginster, J., et al. (1993) Osteoporosis Int., 3:261; Kozono, et al. (1992) Endocrinology, 131(6):2885), against erythropoietin in patients undergoing therapy for chronic renal failure (Casadevall, N., et al. (2002) N. Engl. J. Med., 346(7):469; Gershon et al. (2002) N. Engl. J. Med., 346(20):1584) and against interferon antibodies in individuals undergoing treatment for multiple sclerosis (Deisenhammer, F., et al. (2001) Neurology, 52:1239). In the cases of IFNβ and factor VIII, the percentage of individuals developing neutralizing antibodies is as high as 30-40% (Bristol, et al. (2001) Hum. Gene. Ther., 12(13):1651; Deisenhammer, et al. (2001) Neurology, 52:1239). Furthermore, recent reports regarding the development of antibodies against erythropoietin, which leads to aplasia, provide clear evidence that the development of anti-drug antibodies can cause severe safety problems (Casadevall, N., et al. (2002) N. Engl. J. Med., 346(7):469; Gershon, et al. (2002) N. Engl. J. Med., 346(20):158).

Calcitonin is used for treatment of Paget's disease, hypercalcemia and osteoporosis and is a 32 amino acid polypeptide derived from salmon origin. Salmon calcitonin is preferred for therapeutic use over human calcitonin since it is 50-100 times more potent. Salmon calcitonin differs from human calcitonin in 17 out of 32 amino acids. These differences generate a protein that is seen as foreign by the human immune system. Consequently, the administration of salmon calcitonin results in the formation of neutralizing antibodies in a large number of patients taking this drug (Kozono, et al. (1992) Endocrinology, 131(6):2885). The antibody response causes some patients to either become resistant to the treatment or results in other side effects (Muff, et al. (1991) Osteoporosis Int., 1(2):72). Several chimeric human and salmon analogs have been described with biological activity comparable to salmon calcitonin, however, it is not clear if the use of these chimeric molecules reduces the formation of neutralizing antibodies (Kozono, et al. (1992) Endocrinology, 131(6):2885); Maier, et al. (1976) Endocrinology, 5(suppl), 327s).

Human erythropoietin is a heavily glycosylated endogenous protein used for the treatment of anemia in patients with chronic renal failure. Some of the commercially available products include various forms of erythropoietin designated, for example, erythropoietin alpha, erythropoietin beta and darbepoietin alpha; these products differ from each other in their glycosylation patterns. Recent reports have demonstrated that treatment with recombinant erythropoietin can result in pure-red cell aplasia, causing a significant safety risk (Casadevall. N., et al (2002) N. Engl. J. Med., 346(7):469; Gershon, et al. (2002) N. Engl. J. Med., 346(20):158; Mercadal, et al. (2002) Nephrol. Dial. Transplant, 17 (5):943). Moreover, it has been established that the development of aplasia was due to the formation of anti-erythropoietin antibodies. These antibodies cross-reacted with all known brands of erythropoietin and also recognized deglycosylated erythropoietin, suggesting that the reactivity was targeted against the protein moiety of the molecule. Since the initial report and within the last 1-2 years, several new cases of aplasia have been reported and are being characterized, highlighting the immediacy and importance of addressing this issue in patients undergoing erythropoietin therapy.

Interferon beta 1_{b} (IFNβ) therapy is an effective treatment for patients with relapsing-remitting Multiple Sclerosis. One disadvantage of this treatment is the occurrence of antibodies against IFNβ that inhibit its biological activity (Deisenhammer. F., et al. (2001) Neurology, 52:1239). These antibodies are neutralizing in nature and patients with these neutralizing antibodies respond to IFNβ less well than patients without antibodies (Abdul-Ahad, et al. (1997) Cytokines Cell Mol. Ther., 3(1):27). The two commercially available forms are the glycosylated IFN-β₁ₐ and the non-glycosylated IFN-β_{1b}. Both forms induce the production of neutralizing antibodies, however, the IFN-β_{1b} molecule is reported to be more immunogenic than the IFNβ₁ₐ molecule (Fernandez, et al. (2001) J. Neurol., 248:383). Although not proven, this difference may be due to the chemical structure of the former, which can produce aggregates that enhance antibody production.

Human growth hormone (“hGH”) is a 191 amino acid protein produced by recombinant DNA technology for therapeutic uses including, skeletal growth in children and adults with pituitary growth hormone deficiency, various metabolic disorders that ultimately result in growth retardation, chronic renal insufficiency, and Turner syndrome. Thirty percent of patients develop antibodies to hGH. Although these antibodies are claimed to be non-neutralizing, anecdotal problems have been reported.

Insulin is a 58 amino acid protein, consisting of alpha and beta chains with several inter and intrachain disulphide bonds. Insulin is used for treatment of Type I and Type II diabetes. A significant number of patients receiving insulin via a pulmonary route have developed antibodies. Moreover, development of antibodies to insulin has also been reported in a small number of patients taking the drug subcutaneously. While the clinical significance of these antibodies is unclear, the findings have caused a concern with the FDA authorities and IND filing of the product from hale/Pfizer has been put on hold.

As mentioned above, the field of protein and antibody therapeutics is also undergoing a revolutionary change with the advent of protein modification technologies aimed at improving therapeutic proteins. To this effect, several different approaches have been undertaken to analog proteins to improve their pharmacological properties. However, several studies have demonstrated that even a single amino acid residue change can dramatically increase the immunogenicity of a protein molecule (Maizels, et al. (1980) Eur. J. Immunol., 10(7):509). Moreover, it has been demonstrated that T lymphocyte reactivity is focused on the region immediately adjacent to the changed amino acid and is not due to any conformational effect (Grewal, I., et al. (1995) PNAS, 92(5):1779). Because these immunogenic regions correspond to short linear stretches of the protein sequence, they can be modified rationally to reduce or eliminate immunogenicity with minimal impact on the structure or function of the molecule. The technology based on this approach, comprises identifying an MHC class II epitope in a protein or antibody drug, and modifying the epitope so that it will no longer elicit a class II-mediated immune response. This technology has been termed “ImmunoStealth™”.

Monoclonal antibodies can also be powerful immunogens. If antibodies of murine origin are administered to patients, a human anti-mouse response promptly develops leading to inactivation or decreased efficacy of the monoclonal antibody drug (Siegel, (2002) Tranfus. Clin. Biol., 9(1):15). These results provide a clear demonstration that development of anti-drug antibodies can lead to decreased drug efficacy.

Indeed, the immunogenicity of mouse monoclonal antibodies has prompted the development of new technologies aimed at overcoming the problem. Chimeric and humanized antibodies have been developed, in which the immunoglobulin backbone is of human origin, but the variable regions are either of mouse origin, or fully human, respectively. Despite these approaches, neutralizing antibodies (human anti-chimeric antibodies; HACA, or human anti-human antibodies; HAHA) develop in many instances (Tcheng, J., et al. (2001) Circulation, 104(8):870; Ritter, et al. (2001) Cancer Research, 61(18):6851).

Even with only minor differences in the CDR region or V region sequences, an immune response can develop against the antibody. While the human immune system is normally physiologically exposed to large amounts of circulating human immunoglobulin (Ig), high concentrations of antibody molecules all carrying the same unique CDR regions are detected as a perturbation of immune homeostasis. Therefore, the use of chimeric and humanized antibodies is not expected to eliminate unwanted immune responses directed against the antibody therapeutics. A recent example of the immunogenic potential of humanized antibodies has been exemplified by the MEDI-507 humanized antibody from Medimmune which is in clinical trials for treatment of psoriasis. It has been reported that 50% of patients treated with the humanized antibody in Phase II testing showed immunogenicity to MEDI-507 (BioCentury Extra, Oct. 24, 2002). Furthermore, even fully human antibodies generated in transgenic mice might be capable of eliciting an immune response as high concentrations of unique CDR regions in such antibodies may not be tolerated by the immune system.

Because the field of human and humanized antibody therapy is still in its infancy, there is a need for the development of technologies aimed at disruption of undesired immune responses. The present invention describes a technology aimed at addressing some of these and other needs. This ImmunoStealth™ technology is based on the disruption of molecular mechanisms involved in the development of antibody responses. In certain embodiments, the present invention may be used to identify immunodominant T helper epitopes within proteins of interest using an integrated bioinformatic, biochemical, and cellular immunological approach. The identification, and subsequent modification, of such epitopes will aid in the reduction of immunogenicity of proteins and antibodies used in a therapeutic capacity. Moreover, various embodiments of the invention will enable the design of highly effective and potent vaccines that exhibit a reduced immunogenicity when compared to their unmodified parent molecules.

The basis for the initial immunological response to many therapeutic proteins and antibodies is the recognition of peptide fragments of these molecules as “foreign” by the immune system, and the accompanying activation of specific helper T lymphocytes (HTL) that, in turn, direct the formation of antibodies against the therapeutic protein or antibody. The antibodies may then bind and neutralize the therapeutic protein or antibody. The result is a decreased efficiency or a complete inactivation of the therapeutic molecule. Various other immunological responses including, but not limited to, allergic reaction, are also frequently associated with the formation of anti-therapeutic molecule antibodies. Accordingly, certain embodiments of the present invention are directed to the discovery that immune reactivity within a protein may generally be ascribed to one or more immunodominant epitopes, and that the identification and modification of such epitopes will lead to a decrease in the immune response thereto.

Other technologies have attempted to address the same issues, but have been largely unsuccessful. For example, the process of adding polyethylene glycol moieties to a protein or antibody (termed “PEGylation”) aims to improve pharmacokinetics and to potentially reduce the immunogenicity of proteins (Hinds, K. D., et al. (2002) Adv. Drug Deliv. Rev. 54(4):505; Chen, A. M., et al. (2001) Bio. Drugs 15(12):833) by providing an “immunocamouflage.” This approach has been used to generate compounds with reduced immunogenicity (Chen, A. M., ibid.; Hu, R. G., et al. (2002) Int. J. Biochem. Cell Biol. 34(4):396). However, some PEGylated molecules may have deleterious side effects when used therapeutically. Consequently, such PEGylated molecules have thus far failed development. A second example has been designated “exon shuffling.” The process exon shuffling alters the position of protein coding regions or domains within a full-length protein in order to select for improved characteristics. Currently, no therapeutic protein or antibody modified as a result of exon shuffling technology has yet risen to the level of a clinical trial. Antibody therapy with humanized anti-CD4 antibodies is an additional example of a technology that has unsuccessfully pursued some of the same ends of the present invention. The potential success of such an approach is further complicated by the fact that such antibodies would have to be co-administered with the therapeutic protein or antibody.

### SUMMARY OF THE INVENTION

The present invention is based, at least in part, on the discovery and validation of specific motifs and assay systems for quantitative binding affinity measurements for HTL epitopes against various DR and DQ molecules, representative of the worldwide population. The present invention validates the use of various human in vitro cellular assays for the detection of immunodominant epitopes. The invention also provides a means of reducing the immunogenicity of therapeutic drugs (including, but not limited to, therapeutic proteins and antibodies) through an integrated approach that uses a combination of the above described methods.

Definitions

The term “peptide” is used interchangeably with “oligopeptide” in the present specification to designate a series of residues, typically L-amino acids, connected one to the other typically by peptide bonds between the alpha-amino and carbonyl groups of adjacent amino acids. The oligopeptides of the invention are less than about 50 residues in length and usually consist of between about 10 and about 30 residues, more usually between about 12 and 25, and often 15 and about 20 residues. The oligopeptides or peptides can be a variety of lengths, either in their neutral (uncharged) forms or in forms which are salts, and either free of modifications such as glycosylation, side chain oxidation, or phosphorylation or containing these or other modifications, subject to the condition that, although the modification may alter the biological activity of the polypeptides described herein, the modification may not destroy the biological activity of these polypeptides.

Throughout this disclosure, results are expressed in terms of IC50's. Given the conditions in which the assays are run (i.e., limiting MHC and labeled peptide concentrations), these values approximate K_{D} values. It should be noted that IC50 values can change, often dramatically, if the assay conditions are varied, and depending on the particular reagents used (e.g., MHC preparation, etc.). For example, excessive concentrations of MHC will increase the apparent measured IC50 of a given ligand. An alternative way of expressing the binding data is a relative value to a reference peptide. The reference peptide is included in every assay. As a particular assay becomes more or less, sensitive, the IC50's of the peptides tested may change somewhat. However, the binding relative to the reference peptide will not change. For example, in an assay run under conditions such that the IC50 of the reference peptide increases 10-fold, all IC50 values will also shift approximately 10-fold. Therefore, to avoid ambiguities, the assessment of whether a peptide is a good, intermediate, weak or negative binder should be based on its IC50, relative to the IC50 of the standard peptide. The threshold affinity associated with immunogenicity in the context of DR molecules has been previously defined to be 1000 nM (U.S. Pat. No. 6,413,517 B1, incorporated by reference in its entirety). A “degenerate” binding peptide is defined as a peptide that has a binding affinity equal to greater than 1000 nM (i.e., a binding affinity represented by 1000 nM or less) against at least 33% of the MHC molecules tested (e.g., if binding is measured against 17 molecules, a degenerate peptide must bind to at least 5 out of the 15 molecules with a binding affinity of 1000 nM or less).

An “immunogenic peptide” is a peptide which comprises an allele-specific motif such that the peptide will bind an MHC molecule and induce an HTL response. Immunogenic peptides of the invention are capable of binding to an appropriate HLA molecule and inducing HTL response against the antigen from which the immunogenic peptide is derived. Peptides having immunogenic properties can be modified as necessary to provide certain desired attributes, e.g., disruption of immunogenic properties against the appropriate T cell, and decrease in binding affinity against one or more MHC molecules. For instance, the peptides may be subject to various changes, such as substitutions where one amino acid residue is replaced by another to affect the MHC binding capacity of the peptide. Amino acid substitutions are often of single residues. However, multiple substitutions, insertions, deletions or any combination thereof may be combined to arrive at a final peptide. Such modified peptides may be referred to as “analog” peptides.

A “conserved residue” is a conserved amino acid occupying a particular position in a peptide motif; typically, but not always, a position where the MHC structure may provide a contact point with the immunogenic peptide. One to three, typically two, conserved residues within a peptide of defined length defines a motif for an immunogenic peptide. These residues are typically in close contact with the peptide binding groove, with their side chains buried in specific pockets of the groove itself.

The term “motif” refers to the pattern of residues of defined length, usually between about 8 to about 11 amino acids, which is recognized by a particular MHC allele. The term “supermotif” refers to motifs that, when present in an immunogenic peptide, allow the peptide to bind more than one HLA antigen. The supermotif preferably is recognized by at least one HLA allele having a wide distribution in the human population, preferably recognized by at least two alleles, more preferably recognized by at least three alleles, and most preferably recognized by more than three alleles.

The phrases “isolated” or “biologically pure” refer to material which is substantially or essentially free from components which normally accompany it as found in its native state. Thus, the peptides of this invention do not contain materials normally associated with their in situ environment, e.g., MHC I molecules on antigen presenting cells. Even where a protein has been isolated to a homogenous or dominant band, there may be trace contaminants in the range of 5-10% of native protein which co-purify with the desired protein. In preferred embodiments, isolated peptides of this invention do not contain such endogenous co-purified protein.

The term “residue” refers to an amino acid or amino acid mimetic incorporated in an oligopeptide by an amide bond or amide bond mimetic.

The term “fragment” as applied to polynucleotides of the invention, refers to some portion of the reference polynucleotide less than its full length. For example, a fragment of a 100 bp polynucleotide may be of any length from several contiguous nucleotides to 99 contiguous nucleotides of the reference 100 bp polynucleotide. That is to say, the length of the fragment may be any number of nucleotides expressed as any whole integer from several to, and including, 99 contiguous nucleotides of the 100 bp reference polynucleotide. In preferred embodiments, “several” is equal to 5, 6, 7, 8, 9, 10, 11, 12, 13, 14, 15, 16, 17, 18, 19 or 20 contiguous nucleotides of the reference polynucleotides. In additional preferred embodiments, a fragment is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length. In more preferred embodiments, a fragment is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In even more preferred embodiments a fragment is 9, 12, 15, 18, 21, 24, 27, 30, 33, 36, 39, 42, 45, 48 or 51 nucleotides in length. In certain embodiments, a fragment need not be free-standing. For example, 2 or more fragments of the same reference polynucleotide may be joined end-to-end in a single polynucleotide molecule in such embodiments, additional polynucleotide sequences may also be present, such as, for example, linkers, spacers, restriction endonuclease recognition sequences, vector sequences, and the like.

### BRIEF DESCRIPTION OF THE DRAWINGS AND TABLES

FIG. 1 provides an illustration of the ImmunoStealth™ process.

FIG. 2 illustrates an epitope bound to its HLA Class II molecule.

FIG. 3 shows an example of an HLA-DR motif. In one embodiment, peptides that correspond to this motif have an F, M, Y, L, 1, V or W at position 1 (relative to the N-terminus); an M at position 2; a T at position 3; a W at position 4; an 1 at position 5; a V, S, T, C, P, A, L, I, V or F at position 6; an M, H or R at position 7; any amino acid at position 8; and an M, H, W, D or E at position 9 (expressed as SEQ ID NO:1). In another embodiment, peptides that correspond to this motif have an F, M or Y at position 1; an M at position 2; a T at position 3; any amino acid at position 4; an I at position 5; a V, S or T at position 6; an M or H at position 7; any amino acid at position 8; and an M or H at position 9 (expressed as SEQ ID NO:2).

FIGS. 4A-E. Predicted IC50 (PIC) versus Measured IC50 for DRB1*0101: FIG. 4 depicts the predicted binding by our algorithm versus measured binding affinity data for each of the following proteins: human erythropoietin (“EPO”) (amino acids 28-193 of P01588) (SEQ ID NO:3), salmon calcitonin (“Calcitonin”) (amino acids 83-114 of P01263) (SEQ ID NO:4), human growth hormone 1 isoform 1 (“hGH”) (amino acids 27-217 of P01241) (SEQ ID NO:5), human insulin alpha (amino acids 90-110 of P01308) (SEQ ID NO:6) and human insulin beta (amino acids 25-54 of P01308) (SEQ ID NO:7) (alpha and beta collectively, “insulin”), and human interferon beta (“IFNb”) (amino acids 22-187 of AAC41702) (SEQ ID NO:8). The lower the IC50 value represents a higher measured binding affinity or a greater chance that a predicted epitope will bind. For all other predictive algorithms, peptides with higher values have a greater likelihood of binding. To reflect this, measured binding and PIC results were normalized by performing 1/IC50 calculations. The output of each algorithm (for each protein) is then plotted against the results of binding to DRB1*0101, using overlapping 15mer peptides.

FIG. 5. Regression Analysis of Algorithm Prediction versus Binding: FIG. 5 illustrates the ability of each algorithm to predict binding to DRB1*0101 as compared to measured binding. Each of the following proteins: salmon calcitonin (amino acids 83-114 of P01263) (SEQ ID NO:4), human erythropoietin (amino acids 28-193 of P01588) (SEQ ID NO:3), human growth hormone 1 isoform 1 (amino acids 27-217 of P01241) (SEQ ID NO:5), human insulin alpha (amino acids 90-110 of P01308) (SEQ ID NO:6), human insulin beta (amino acids 25-54 of P01308) (SEQ ID NO:7), and human interferon beta (amino acids 22-187 of AAC41702) (SEQ ID NO:8) were analyzed together using PIC, Propred, SYFPEITHI and MHC Thread. The algorithm results are plotted versus the measured binding data to DRB1*0101.

FIG. 6. Binding Characteristics of Peptides: Overlapping 15mer peptides from each of the following proteins were synthesized: salmon calcitonin (amino acids 83-114 of P01263) (SEQ ID NO:4), human erythropoietin (amino acids 28-193 of P01588) (SEQ ID NO:3), human growth hormone 1 isoform 1 (amino acids 27-217 of P01241) (SEQ ID NO:5), human insulin alpha (amino acids 90-110 of P01308) (SEQ ID NO:7), human insulin beta (amino acids 25-54 of P01308) (SEQ ID NO:6), and human interferon beta (amino acids 22-187 of AAC41702). (SEQ ID NO:8) These peptides were tested for binding against a panel of MHC class II molecules. FIG. 6 illustrates the quantity of peptides binding to the MHC class II molecules.

FIG. 7. Degenerate Regions in Candidate Proteins: Peptides from human erythropoietin (“EPO”) (amino acids 28-193 of P01588) (SEQ ID NO:3), human growth hormone 1 isoform 1 (“huGH”) (amino acids 27-217 of P01241) (SEQ ID NO:5), salmon calcitonin (“SCalci”) (amino acids 83-114 of P01263) (SEQ ID NO:4), human insulin alpha (amino acids 90-110 of P01308) (SEQ ID NO:6), human insulin beta (alpha and beta collectively “insulin”) (amino acids 25-54 of P01308) (SEQ ID NO:7), and human interferon beta (“IFNb”) (amino acids 22-187 of AAC41702) (SEQ ID NO:8) are plotted based on the numbers of MHC molecules bound. A threshold of molecules bound is depicted in the graph.

FIGS. 8A-B. Antigenicity analysis of overlapping EPO peptides: Overlapping peptides spanning the entire EPO sequence were analyzed for antigenicity against T cell lines generated against whole EPO protein. The magnitude (10A) and frequency (10B) of ELISPOT responses obtained against each peptide are shown.

FIG. 9. Immunodominant EPO peptides are presented by DR molecules: Single transfected fibroblast or EBV transformed cells carrying appropriate MHC molecules corresponding to the donor were used to determine how well the immunodominant EPO-101 peptide is presented by the relevant HLA DR molecules. Presentation of the EPO-101 peptide by fibroblasts and EBV cells are shown. Two analogs, EPO101P and EPO101D that do not bind the DR molecules were also tested in this experiment and the results are shown.

FIGS. 10A-F. Recommended EPO molecules with potentially reduced immunogenicity: Results from all the above sections were analyzed and recommendations for the generation of modified EPO proteins generated are indicated. The recommendations were based on binding analysis, antigenicity, structural analysis, targeting of both the immunodominant EPO regions and non-creation of any additional DR3 MHC binding sites. FIG. 10A shows the amino acid sequence of the wild type EPO sequence (SEQ ID NO:9). FIGS. 10B-F show the amino acid sequences of Modified Human Erythropoietin-constructs 1-5, respectively (SEQ ID NOs:10-14, respectively).

FIG. 11 depicts amino acid sequences of polypeptides.

FIG. 12. Immunogenicity of wild-type and analog peptide combinations. Immunogenicity of the two wild-type EPO epitopes (EPO 101-115 and EPO 136-150) in the form of synthetic peptides, or EPO epitope analog combinations C2 (L102P and S146D), C3 (T107D and S146D) C4 (L102G, T107D and S146D) and C5 (L102S, T107D and S146D) were tested in primary in vitro induction assays. Ten individual cultures each from five different donors were tested. All the data points are plotted as net SFC/5×10⁴ effector cells.

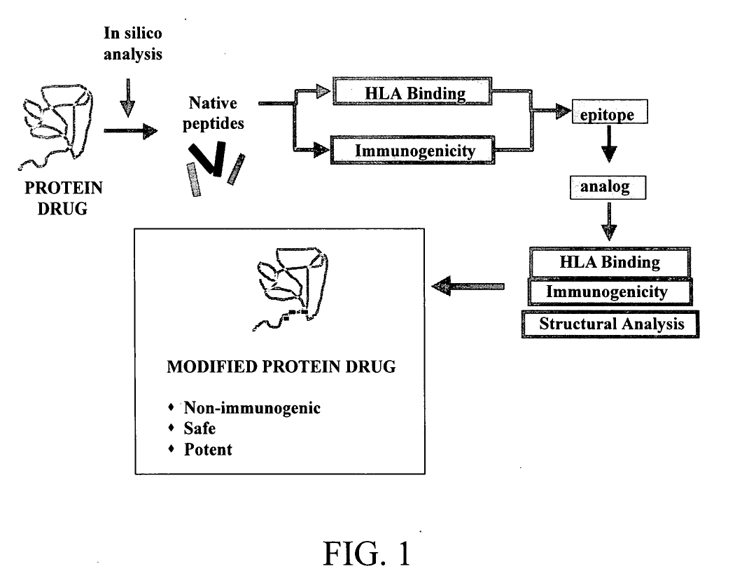

Table 1 illustrates the high degree of polymorphism in HLA-DR molecules, and the representation of each allele in several major ethnic groups.

Table 2 depicts the binding of immunodominant epitopes by multiple DR molecules and shows representative binding data for immunodominant epitopes derived from Tetanus Toxin (“Tet Tox 830”) (Panina-Bordignon, et al. (1989) Eur. J. Immunol, 19(12):2237) (SEQ ID NO:15), Influenza Haemagglutinin (“HA 307”) (Rothbard, et al. (1988) Cell, 52(4):515) (SEQ ID NO:16), Hepatitis C Virus NS3 Protein (“HCV NS3 1242”) [Diepolder, et al. (1997) J. Virol., 71(8):601] (SEQ ID NO:17), Hepatitis B Virus Polymerase Protein (“HBV POL 412”) (Ferrari C., et al. (1991) J. Clin. Invest., 88(1):214) (SEQ ID NO:18) and Plasmodium falciparum (P. fal. SSP2.61”) (Doolan, et al. (2000) J. Immunol., 165(2):1123) (SEQ ID NO:19). In the Table, a hyphen (“−”) indicates that no appreciable binding was observed, and shading indicates that binding with an IC50>1000 nM was observed.

Table 3. A summary of candidate molecules considered for validation of the ImmunoStealth™ technology. Several different criteria (recited in the column labeled “PARAMETERS”) were analyzed for selecting which of these candidates would be further validated.

Table 4. Efficiency of Predictive Algorithms for DRB1*0101 Binding: The efficiency (Number of binders/total number of peptides synthesized) at several algorithm sensitivity levels was calculated.

Tables 5A-E. PIC and Measured Binding for Overlapping Peptides from EPO, hGH, Calcitonin, IFNb and Insulin: The PIC algorithm was performed using each of the overlapping 15mer peptides from EPO (Table 5A; SEQ ID NOs:20-50), Calcitonin (Table 5B; SEQ ID NOs:51-56), hGH (Table 5C; SEQ ID NOs:57-82), IFNb (Table 5D; SEQ ID NOs:83-113) and Insulin (Table 5E; SEQ ID NOs:114-122). Binding to 15 different MHC class II molecules was also tested using the 15mer peptides. The results are shown in Tables 5A-E including a crossreactivity column (labeled “xrn (1000 nM)”), representing the number of MHC class II molecules which the peptide bound at a threshold of 1000 nM or less.

Table 6. Binding Capacity of Known HLA-DR and DQ restricted epitopes: Table 6 depicts known Class II-restricted epitopes and their binding affinity to fifteen different MHC class II molecules (SEQ ID NOs:123-151). These epitopes and their HLA-restriction are described in the literature (See, e.g., Anderson, D. C., et al. (1988) Science, 242:259-61; Bocchia, M., et al. (1996) Blood, 87:3587-92; Celis, E., et al. (1988) J. Immunol, 141:2721-28; Dayan, C., et al (1991) Proc. Natl. Acad. Sci. U.S.A., 88:7415-19; De Magistris, M. T., et al. (1989) J. Exp. Med., 169:1519-32; Ferrari, C., et al. (1989) J. Clin. Invest., 84:1314-1319; Lamonaca, V., et al. (1999) Hepatology, 30:1088-98; Markovic-Plese, S., et al. (1995) J. Immunol., 155:982-92; Oftung, F., et al. (1988) J. Immunol., 141:2749-54; Oftung, F., et al. (1990) J. Immunol., 144:1478-83; Panina-Bordignon, P., et al. (1989) Eur. J. Immunol., 19:2237-42; Perez, M., et al., (1990) J. Biol. Chem., 265:16210-15; Rothbard, J. B. and Taylor, W. R. (1988) EMBO J., 7:93-100; Rothbard, J. B., et al. (1988) Cell, 52:515-23; Sinigaglia, F., et al. (1988) Nature, 336:778-80; Topalian, S. L., et al. (1996) J. Exp. Med., 183:1965-71; Valli, A., et al. (1993) J. Clin. Invest., 91:616-28; and Van Schooten, W. C., et al. (1989) Eur. J. Immunol., 19:2075-79) The binding capacity and cross-reactivity of these peptides were tested against a panel of 15 HLA Class II molecules. The epitopes tested were positive for the previously described restriction, in which 1000 nM or less peptide concentration was required for binding (see Doolan, D. L., et al. (2000) J. Immunol., 165:1123-37; Wilson, C. C., et al. (2001) J. Virol., 75:4195-4207). In addition to the reported restriction, the peptides tested bound additional class II molecules. On average, cross-reactivity encompassed 6 out of the 15 Class II molecules tested. Therefore, HLA Class II-restricted epitopes are cross-reactive and mutations in molecule composition should modify the binding to not just the known restriction but also to the other molecules to which the peptide can bind. If class II peptides were not cross-reactive, immunogenicity in the general population would not be affected as efficiently by a therapeutic molecule modified according to the ImmunoStealth process as described herein.

Table 7. Binding Capacity of Cross-reactive Peptides: Peptides from EPO, hGH, and IFNb, and which bind 5 or more MHC Class II molecules are shown. Peptides that bind 8 or more MHC molecules are shown in dark shading. Peptides that bind 6 or 7 molecules are shown in light shading. Peptides that bind 5 MHC are shown with no shading.

Tables 8A-B. Immunogenicity of Candidate Molecules: Immunogenicity of hGH and Erythropoietin was evaluated using in vitro systems. MHC typing of the donors used in the study was carried out and the results are detailed in Table 8A. The results of the immunogenicity analyses are shown in Table 8B. Cell lines were tested for immunogenicity in standard IFNδ ELISPOT assays. A positive immune response is P<0.05.

Table 9. Correlation of Antigenic EPO peptides with binding and PIC analysis. A summary of the antigenic peptides from the EPO protein is shown. A determination of how many of these peptides are predicted by binding and PIC analysis is indicated. The criteria for a “+” antigenic response is a positive response in ≧2 donors, a “+” for binding is one where the peptide bound ≧5 alleles with an IC50≦1000 nM and a “+” for PIC is a peptide with a score of ≦125 nM.

Table 10A-B. Analog Binding Results. The binding results for each analoged peptide according to MHC molecule is shown. Peptide analogs based on the EPO-101 peptide are shown in Table 10A. Peptide analogs based on the EPO-136 peptide are shown in Table 10B. The bound column represents the numbers of MHC class II molecules bound by each peptide at a threshold of 1000 nM. The comp ratio depicts the comparative binding ratio of the bound/tested compared to the wild-type peptide (comp ratio=1.000). The 10× red column represents the numbers of MHC molecules for which peptide binding affinity of the analog was reduced. The selected candidate column depicts the analogs for which there were at least 5 molecules which had reduced binding affinity and the comparative ratio was 1.000 or less. In the Table, a hyphen (“−”) indicates that no appreciable binding was observed, while a blank space indicates that the combination was not tested.

Table 11A-B. Binding Analysis of EPO Analogs with Reduced Binding Capacity. Analogs for EPO 101 (Table 11A) and EPO 136 (Table 11B) which had a ten-fold reduction in 5 or more MHC molecules are shown. Binding against all the 15 DR and DQ molecules is shown. The 10× red column represents the numbers of MHC molecules for which peptide binding affinity of the analog was reduced. The wild type EPO101 and EPO136 peptides are highlighted with light shading.

Table 12. Antigenicity Analysis of EPO Analogs with Reduced Binding Capacity. The analogs identified above were tested in an antigenicity assay against T cell lines from 4 different donors. The results are shown.

Table 13A-B. Structural analysis of functionally important domains in erythropoietin. A literature and conservation analysis of the various single amino acid substitutions reported was compiled and an analysis was carried out to determine the impact of various substitutions on biological activity of the protein (Table 13A). Specific amino acids substitutions identified in Table 12 were subjected to structural modeling and recommendations on the most favorable substitutions to generate were provided (Table 13B).

Table 14A-C. Improving analoging strategies. Several double amino acid substitution analogs were generated and binding analyses were done. The results were compared to the binding analyses of the corresponding single analogs. The 10× red column represents the numbers of MHC molecules for which peptide binding affinity of the analog was reduced. Table 14A shows the results of double analogs generated from the EPO-101 degenerate region. Table 14B shows the results of double analogs generated from the EPO-136 degenerate region. The number of MHC molecules bound by the double analog and the number of molecules in which the binding affinity was decreased by 10-fold or more are shown in Table 14C. An antigenicity analysis of the double analogs identified was carried out in four different donor lines. The responses obtained are shown.

### DESCRIPTION OF THE PREFERRED EMBODIMENT

The present invention relates to compositions and methods for removing undesired HTL epitopes which elicit CD4 responses against a therapeutic protein or antibody. In certain embodiments, the modified epitopes do not elicit a CD4 response at all. In other embodiments, the modified epitopes elicit a reduced CD4 response as compared to the wild type epitope. The present invention also relates to compositions and methods for preventing, treating or diagnosing a number of pathological states such as viral, fungal, bacterial and parasitic diseases and cancers. In a preferred embodiment, a therapeutic protein or antibody is analyzed for the presence of HTL epitopes (immunodominant HTL epitopes, in a highly preferred embodiment); the epitopes are modified according to methods of the invention; and the resulting modified protein is used therapeutically. In particular, one embodiment of the present invention provides a novel molecule generated by the above method which does not bind, or binds at a reduced level, to selected major histocompatibility complex (“MHC”) class II molecules, and thereby fails to induce an immune response or induces a reduced immune response. Another embodiment of the invention provides a second novel molecule that is capable of binding selected MHC class II molecules and actually inducing an immune response.

Peptide binding to MHC molecules is determined by the allelic type of the MHC molecule and the amino acid sequence of the peptide. MHC class I-binding peptides usually contain within their sequence two conserved (“anchor”) residues that interact with corresponding binding pockets in the MHC molecule. Specific combination of anchor residues (usually referred to as “MHC motifs”) required for binding by several allelic forms of human MHC (HLA, histocompatibility leukocyte antigens) are described in International Application Publication Nos. WO 94/03205 and WO 94/20127. Definition of specific MHC motifs allows one to predict from the amino acid sequence of an individual protein, which peptides have the potential of being immunogenic for CTL. These applications describe methods for preparation and use of immunogenic peptides in the treatment of disease.

The peptides described here can also be used as helper T peptides either alone or in combination with peptides which induce a CTL response. Such combinations and their uses are described in International Application Publication No. WO 95/07077.

The DR- or DQ-binding peptides or nucleic acids encoding them may be used to treat a variety of diseases and conditions involving unwanted T cell reactivity. That is, modification of DR- or DQ-binding peptides can be used to generate proteins or antibodies with reduced immunogenicities that may be used therapeutically more successfully than their unmodified parent molecules. A non-limiting list of examples of diseases and conditions that can be treated using DR- or DQ-binding peptides includes: autoimmune diseases (e.g., rheumatoid arthritis, multiple sclerosis, and myasthenia gravis), allograft rejection, allergies (e.g., pollen allergies), Lyme disease, Hepatitis B, Hepatitis C, LCMV, post-streptococcal endocarditis, or glomerulonephritis, Ulcerative colitis, Crohn's disease, psoriasis, chronic renal failure, asthma, breast cancer, non-Hodgkin's lymphoma, transplantation, hemophilia, multiple sclerosis, Paget's disease, osteoporosis, chronic granulatomous disease, genital warts, anemia, diabetes, defective tissue growth, metabolic diseases, and food hypersensitivities.

The subject invention provides isolated and/or purified and/or recombinant polynucleotides encoding the ImmunoStealth polypeptides, peptides, and/or proteins of the subject invention. Thus, the present invention provides isolated and/or purified polynucleotide sequences comprising:
a. a polynucleotide sequence encoding a polypeptide sequence selected from the group consisting of SEQ ID NO: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244;
b. a complementary polynucleotide sequence to a polynucleotide sequence encoding a polypeptide sequence selected from the group consisting of SEQ ID NO: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244;
c. a polynucleotide sequence having at least about 20% to 99.99% identity to a polynucleotide sequence of (a) or (b); or
d. a fragment of a polynucleotide sequence according to (a) or (b).

The subject invention also provides a number of non-limiting embodiments and aspects that include:

A) Peptides, polypeptides, proteins, and/or antibodies having reduced immunogenicity as compared to the naturally occurring form of the peptide, polypeptide, or protein while retaining at least (or at least about) 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of the biological activity of the unmodified (or naturally occurring) molecule;

B) Peptides, polypeptides, and/or proteins according to embodiment A, wherein the peptide, polypeptide, or protein is a therapeutic peptide, polypeptide, protein or antibody used in the diagnosis or treatment of diseases, conditions, and/or disorders;

C) Peptides, polypeptides, proteins, and/or antibodies according to embodiment A or B, wherein peptide, polypeptide, or protein comprises all, or a portion of, IL-1, IL-2, IL-3, IL-4, IL-5, IL-6, IL-7, IL-8, IL-9, IL-10, IL-11, IL-15, IL-16, IL-18, IL-19, IL-23, IL-24, erythropoietin, insulin, human growth hormone, calcitonin (e.g., salmon calcitonin, human calcitonin), Factor VIII, G-CSF, M-CSF, GM-CSF platelet derived growth factor (PDGF), MSF, FLT-3 ligand, EGF, fibroblast growth factor (FGF; e.g., aFGF (FGF-1), bFGF (FGF-2), FGF-3, FGF-4, FGF-5, FGF-6, or FGF-7), human insulin alpha, human insulin beta, insulin-like growth factors (e.g., IGF-1, IGF-2); vascular endothelial growth factor (VEGF; e.g., VEGF, VEGF-2, VEGF-3, VEGF-4, VEGF-A, VEGF-B, VEGF-C, VEGF-D); interferons (e.g., IFN-D, IFN-γ, IFN-α); leukemia inhibitory factor (LIF); ciliary neurotrophic factor (CNTF); oncostatin M; stem cell factor (SCF); transforming growth factors (e.g., TGF-α, TGF-β1, TGF-β1, TGF-β1), or chemokines (such as, but not limited to, BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP4, MDC/STCP-1, ABCD-1, MIP-1α, MIP-1β, MIP-2α/GROβ, MIP-3α/Exodus/LARC, MIP-3β/Exodus-3/ELC, MIP-4/PARC/DC-CK1, PF-4, RANTES, SDF1α, TARC, TECK, Factor X, PTH, and antibodies (or example, but not limited to, chimeric and humanized antibodies such as REMICADE® (Infliximab; a chimeric α-TNF antibody), REOPRO® (Abciximab; a chimeric antibody that binds to the glycoprotein (GP) IIb/IIIa receptor of human platelets), SIMULECT® (Basiliximab; a chimeric monoclonal antibody (IgG1k) that specifically binds to and blocks the interleukin-2 receptor a-chain (IL-2Ra, also known as CD25 antigen), ZENAPAX® (Daclizumab; a humanized antibody that binds specifically to the Tac subunit of the human high-affinity interleukin-2 (IL-2) receptor that is expressed on the surface of activated lymphocytes), HERCEPTIN® (Trastuzumab; a humanized monoclonal antibody that specifically binds to the extracellular domain of the human epidermal growth factor receptor 2 protein, HER2), SYNAGIS® (Palivizumab; a humanized monoclonal antibody (IgG1k) directed to an epitope in the A antigenic site of the F protein of respiratory syncytial virus (RSV)), XOLAIR® (Omalizumab; a humanized monoclonal antibody designed to inhibit IgE before it has the opportunity to bind to the mast cell), and the like, and wherein the peptide, polypeptide, protein and/or antibody of this embodiment may further comprise an optional linker or tag for the purification of isolation of the polypeptide;

D) Peptides, polypeptides, proteins and/or antibodies according to embodiment A, B, or C, wherein the protein is EPO and comprises a sequence selected from the group consisting of SEQ ID NOs: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244;

E) An EPO variant comprising the sequence of SEQ ID NOs: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244, that has the ability to bind to the EPO receptor and fails to activate the receptor (e.g., erythropoietin with alanine substituted for Arg-103 retains the ability to bind to the receptor, but fails to activate it);

F) An EPO variant comprising the sequence of SEQ ID NOs: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244, that has the ability to bind to the EPO receptor and activate the receptor to a greater extent than the wild type EPO molecule;

G) An EPO variant comprising the sequence of SEQ ID NOs: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244, that has the ability to bind to the EPO receptor and activate the receptor to essentially the same level as does the wild type EPO molecule;

H) Compositions comprising a peptide, polypeptide, protein, and/or antibody according to embodiments A, B, C, D, E, F or G and a carrier or pharmaceutically acceptable excipient (including for example, carriers described in E. W. Martin's Remington's Pharmaceutical Science, Mack Publishing Company, Easton, Pa.)

I) A method of antagonizing an EPO receptor (or methods of treating diseases or conditions associated with over-activation of the EPO receptor) comprising the administration of a composition comprising the EPO variant of embodiment E in amounts sufficient to: 1) block the binding of naturally occurring EPO to its receptor; or 2) reduce the activation levels of the EPO receptor;

J) A method of agonizing an EPO receptor (or methods of treating diseases or conditions associated with under-activation of the EPO receptor) comprising the administration of a composition comprising the EPO variant of embodiments F or G in amounts sufficient to: 1) block the binding of naturally occurring EPO to its receptor; or 2) increase the activation levels of the EPO receptor;

K) Methods of treating anemia or stimulating the hematopoietic or erythropoietic system comprising the administration of a composition comprising a carrier or pharmaceutically acceptable excipient and an EPO polypeptide selected from the group consisting of SEQ ID NOs:10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244 in amounts sufficient to stimulate the system or treat anemia;

L) Isolated, purified, or recombinant polynucleotides encoding a peptide, polypeptide, protein and/or antibody according to embodiments A, B, C, D, E, F or G;

M) An isolated, purified, or recombinant polynucleotide according to embodiment L, further comprising regulatory elements such as promoters, enhancers, and termination sequences;

N) A vector comprising an isolated, purified, or recombinant polynucleotide according to embodiments L or M;

O) A host cell comprising an isolated, purified, or recombinant polynucleotide according to embodiments L, M or N;

P) A method of producing a recombinant peptide, polypeptide, protein and/or antibody according to embodiments A, B, C, D E, F or G comprising the culturing of a host cell according to embodiment L under conditions that allow for the expression of the recombinant peptide, polypeptide, protein and/or antibody according to embodiments A, B, C, D, E, F or G; and/or

Q) The method according to embodiment P, further comprising the isolation of the recombinant peptide, polypeptide, protein and/or antibody from the host cell or culture system.

R) The subject invention also provides for a method for reducing a helper T lymphocyte (HTL) response against a candidate protein comprising: a. selecting a protein; b. analyzing the amino acid sequence of the protein for potential HTL epitopes; and c. modifying the amino acid sequence of the protein by removing the potential HTL epitope and thereby generating an analog protein. In various aspects of this method, the HTL response is eliminated or reduced. The reduction of HTL response against a candidate protein can be on the order of at least (or at least about) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% as compared to the HTL response against the native (or wild-type/unmodified) amino acid sequence of the candidate protein. Candidate proteins in this aspect of the invention include, and are not limited to, those discussed supra in embodiments A, B, C, D, E, F, G, or H. Thus, for example, a candidate protein can be an antibody as disclosed in embodiment C. In various aspects of this embodiment, the potential HTL epitope can be an immunodominant epitope and the analog protein produced by this method retains the biological activity of the candidate protein. Analog proteins made according to this aspect of the invention can retain biological activity (as compared to the biological activity of the native/unmodified/wild-type protein) that is at least (or at least about) 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of the biological activity of the native/unmodified/wild-type protein that was selected for modification according to this aspect of the subject invention. In some embodiments of this aspect of the invention, an analog protein can have higher biological activity than the native/unmodified, wild-type protein.

S) Another aspect of the invention provides for an isolated protein, or compositions thereof, comprising an amino acid sequence selected from the group consisting of: SEQ ID NO:10; SEQ ID NO:11; SEQ ID NO:12; SEQ ID NO:13; SEQ ID NO:14; SEQ ID NO:152; SEQ ID NO:154; SEQ ID NO:155; SEQ ID NO:159; SEQ ID NO:162; SEQ ID NO:181; SEQ ID NO:187; SEQ ID NO:199; SEQ ID NO:225; SEQ ID NO:226; SEQ ID NO:227; SEQ ID NO:228; SEQ ID NO:229; and SEQ ID NO:233. The invention also provides for a protein, or compositions thereof, comprising a peptide having an amino acid sequence selected from the group consisting of: SEQ ID NO:245; SEQ ID NO:246; and SEQ ID NO:247.

The subject invention also provides methods for reducing the immunogenicity of peptides, polypeptides, proteins, and/or antibodies, and preferably peptides, polypeptides, proteins, and/or antibodies used therapeutically in the art (which may also be referred to as “ImmunoStealth™ molecules”). This embodiment of the invention is referred to herein as “ImmunoStealth™”. The subject invention also provides for compositions comprising the ImmunoStealth™ molecules of the invention and carriers or pharmaceutically acceptable excipients.

“The terms nucleotide sequence”, “polynucleotide” “nucleic acid” or “nucleic acid molecule” can be used interchangeably and are understood to mean, according to the present invention, either a double-stranded DNA, a single-stranded DNA or products of transcription of the said DNAs (e.g., RNA molecules). It should also be understood that the present invention does not relate to genomic polynucleotide sequences of the disclosed cytokines, hormones, or chemokines in their natural environment or natural state. The nucleic acid, polynucleotide, or nucleotide sequences of the invention have been isolated, purified (or partially purified), by separation methods including, but not limited to, ion-exchange chromatography, molecular size exclusion chromatography, affinity chromatography, or by genetic engineering methods such as amplification, cloning, subcloning or chemical synthesis.

A homologous polynucleotide or polypeptide sequence, for the purposes of the present invention, encompasses a sequence having a percentage identity with the polynucleotide or polypeptide sequences, set forth herein, of between at least (or at least about) 20.00% to 99.99% (inclusive). The aforementioned range of percent identity is to be taken as including, and providing written description and support for, any fractional percentage, in intervals of 0.01%, between 20.00% and, up to, including 99.99%. These percentages are purely statistical and differences between two nucleic acid sequences can be distributed randomly and over the entire sequence length.

In various embodiments, homologous sequences can exhibit a percent identity of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent with the sequences of the instant invention. Typically, the percent identity is calculated with reference to the full length, native, and/or naturally occurring polypeptide or polynucleotide (e.g., those full-length polypeptides set forth in SEQ ID NOs: 9, 245, 246, 247, and 248). The terms “identical” or percent “identity”, in the context of two or more polynucleotide or polypeptide sequences, refer to two or more sequences or subsequences that are the same or have a specified percentage of amino acid residues that are the same, when compared and aligned for maximum correspondence over a comparison window, as measured using a sequence comparison algorithm or by manual alignment and visual inspection. Preferably, such a substitution is made in accordance with analoging principles set forth, e.g., in co-pending U.S. Ser. No. 09/260,714 filed Mar. 1, 1999 and 09/226,775, filed Jan. 6, 1999 and PCT application number PCT/US00/19774 each of which is hereby incorporated by reference in its entirety.

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, (1988) Proc. Natl. Acad. Sci. U.S.A., 85(8):2444-2448; Altschul, et al. (1990) J. Mol. Biol. 215(3):403-410; Thompson, et al., (1994) Nucleic Acids Res. 22(2):4673-4680; Higgins, et al. (1996) Methods Enzymol. 266:383-402; Altschul, et al. (1990) J. Mol. Biol. 215(3):403-410; Altschul et al. (1993) Nature Genetics, 3:266-272). Sequence comparisons are, typically, conducted using default parameters provided by the vendor or using those parameters set forth in the above-identified references, which are hereby incorporated by reference in their entireties.

A “complementary” polynucleotide sequence, as used herein, generally refers to a sequence arising from the hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. A “complementary” polynucleotide sequence may also be referred to as an “antisense” polynucleotide sequence or an “antisense” sequence, whereas the polynucleotide sequence to which the complementary sequence hybridizes may be referred to as a “sense” or “coding” sequence.

Sequence homology and sequence identity can also be determined by hybridization studies under high stringency, intermediate stringency, and/or low stringency. Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under low, intermediate, or high stringency conditions by techniques well known in the art, as described, for example, in Keller, G. H., M. M. Manak (1987) DNA Probes, Stockton Press, New York, N.Y., pp. 169-170.

For example, hybridization of immobilized DNA on Southern blots with ³²P-labeled gene-specific probes can be performed by standard methods (Maniatis, et al. (1982) Molecular Cloning: A Laboratory Manual, Cold Spring Harbor Laboratory, New York). In general, hybridization and subsequent washes can be carried out under intermediate to high stringency conditions that allow for detection of target sequences with homology to the exemplified polynucleotide sequence. For double-stranded DNA gene probes, hybridization can be carried out overnight at 20-25° C. below the melting temperature (Tₘ) of the DNA hybrid in 6×SSPE, 5× Denhardt's solution, 0.1% SDS, 0.1 mg/ml denatured DNA. The melting temperature is described by the following formula (Beltz, et al. (1983) Methods of Enzymology, R. Wu, L. Grossman and K. Moldave [eds.] Academic Press, New York 100:266-285).
Tm=81.5° C.+16.6 Log[Na⁺]+0.41(%G+C)−0.61(%formamide)−600/length of duplex in base pairs.

Washes are typically carried out as follows:
(1) twice at room temperature for 15 minutes in 1×SSPE, 0.1% SDS (low stringency wash);
(2) once at Tₘ−20° C. for 15 minutes in 0.2×SSPE, 0.1% SDS (intermediate stringency wash).

For oligonucleotide probes, hybridization can be carried out overnight at 10-20° C. below the melting temperature (Tₘ) of the hybrid in 6×SSPE, 5× Denhardt's solution, 0.1% SDS, 0.1 mg/ml denatured DNA. Tₘ for oligonucleotide probes can be determined by the following formula:

Tₘ (° C.)=2(number T/A base pairs)⁺4(number G/C base pairs) (Suggs, et al. (1981) ICN-UCLA Symp. Dev. Biol. Using Purified Genes, D. D. Brown [ed.], Academic Press, New York, 23:683-693).

Washes can be carried out as follows:
(1) twice at room temperature for 15 minutes 1×SSPE, 0.1% SDS (low stringency wash);
2) once at the hybridization temperature for 15 minutes in 1×SSPE, 0.1% SDS (intermediate stringency wash).

In general, salt and/or temperature can be altered to change stringency. With a labeled DNA fragment >70 or so bases in length, the following conditions can be used:

Low: 1 or 2×SSPE, room temperature

Low: 1 or 2×SSPE, 42° C.

Intermediate: 0.2× or 1×SSPE, 65° C.

High: 0.1×SSPE, 65° C.

By way of another non-limiting example, procedures using conditions of high stringency can also be performed as follows: Pre-hybridization of filters containing DNA is carried out for 8 h to overnight at 65° C. in buffer composed of 6×SSC, 50 mM Tris-HC1 (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in pre-hybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaC1 and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. are incorporated herein in their entirety.

Another non-limiting example of procedures using conditions of intermediate stringency are as follows: Filters containing DNA are pre-hybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of intermediate stringency which may be used are well known in the art and as cited in Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. are incorporated herein in their entirety.

Duplex formation and stability depend on substantial complementarity between the two strands of a hybrid and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions and deletions can be produced in a given polynucleotide sequence in many ways, and these methods are known to an ordinarily skilled artisan. Other methods may become known in the future.

It is also well known in the art that restriction enzymes can be used to obtain functional fragments of the subject DNA sequences. For example, Bal31 exonuclease can be conveniently used for time-controlled limited digestion of DNA (commonly referred to as “erase-a-base” procedures). See, for example, Maniatis, et al (1982) Molecular Cloning: A Laboratory Manual, Cold Spring Harbor Laboratory, New York; Wei, et al. (1983) J. Biol. Chem., 258:13006-13512.

The present invention further comprises fragments of the polynucleotide sequences of the instant invention. Representative fragments of the polynucleotide sequences according to the invention will be understood to mean any nucleotide fragment having at least 8 successive nucleotides, preferably at least 12 successive nucleotides, and still more preferably at least 15 or at least 20 successive nucleotides of the sequence from which it is derived. The upper limit for such fragments is the total number of polynucleotides found in the full length sequence. In certain preferred embodiments, polynucleotide fragments of the invention encode polypeptides such as those set forth in Tables (for example, single or double amino acid epitope analogs of EPO peptides G101-Q115 (GARSLTTLLRALGAQ) (SEQ ID NO: 152) and D136-R150 (DTFRKLFGVYSNFLR) (SEQ ID NO: 226) as set forth in the Tables).

In some embodiments, the subject invention includes those fragments capable of hybridizing under various conditions of stringency conditions (e.g., high or intermediate or low stringency) with a nucleotide sequence according to the invention; fragments that hybridize with a nucleotide sequence of the subject invention can be, optionally, labeled as set forth below.

In certain embodiments, the subject invention provides for labeled polynucleotides. Labels suitable for use in these embodiments include, and are not limited to 1) radioactive labels, 2) enzyme labels, 3) chemiluminescent labels, 4) fluorescent labels, 5) magnetic labels, or other suitable labels, including those set forth below. These labels are well known in the art and widely available to the skilled artisan. Likewise, methods of incorporating labels into the nucleic acids are also well known to the skilled artisan.

Also encoded by polynucleotides of the invention are additional, non-coding sequences, including for example, but not limited to introns and non-coding 5′ and 3′ sequences, such as the transcribed, non-translated sequences that play a role in transcription, mRNA processing, including splicing and polyadenylation signals, for example, ribosome binding and stability of mRNA; and additional coding sequences which code for additional amino acids, such as those which provide additional functionalities.

Thus, polynucleotide sequences of the invention may be fused to a marker sequence, such as a sequence encoding a peptide which facilitates purification of the fused polypeptide. In certain preferred embodiments of this embodiment of the invention, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described by Gentz and colleagues (Proc. Natl. Acad. Sci. USA 86:821-824 (1989)), for instance, hexa-histidine provides for convenient purification of the fusion protein. The “HA” tag is another peptide useful for purification which corresponds to an epitope derived from the influenza hemagglutinin protein, which has been described by Wilson and coworkers (Cell 37:767 (1984)).

The polynucleotide sequences according to the invention may also be used in analytical systems, such as DNA chips. DNA chips and their uses are well known in the art and (see for example, U.S. Pat. Nos. 5,561,071; 5,753,439; 6,214,545; Schena, et al. (1996) BioEssays, 18:427-431; Bianchi et al. (1997) Clin. Diagn. Virol. 8:199-208; each of which is hereby incorporated by reference in their entireties) and/or are provided by commercial vendors such as Affymetrix, Inc. (Santa Clara, Calif.). In addition, the nucleic acid sequences of the subject invention can be used as molecular weight markers in nucleic acid analysis procedures.

The subject invention also provides for modified nucleotide sequences. Modified nucleic acid sequences will be understood to mean any nucleotide sequence that has been modified, according to techniques well known to persons skilled in the art, and exhibiting modifications in relation to the native, naturally occurring nucleotide sequence. One non-limiting example of a “modified” nucleotide sequences includes mutations in regulatory and/or promoter sequences of a polynucleotide sequence that result in a modification of the level of expression of the polypeptide. A “modified” nucleotide sequence will also be understood to mean any nucleotide sequence encoding a “modified” polypeptide as defined below.

Another embodiment of the invention provides vectors for the cloning and/or the expression of a polynucleotide sequence taught herein. Vectors of this invention, including vaccine vectors, can also comprise elements necessary to allow the expression and/or the secretion of the said nucleotide sequences in a given host cell. The vector can contain a promoter, signals for initiation and for termination of translation, as well as appropriate regions for regulation of transcription. In certain embodiments, the vectors can be stably maintained in the host cell and can, optionally, contain signal sequences directing the secretion of translated protein. These different elements are chosen according to the host cell used. Vectors can integrate into the host genome or, optionally, be autonomously-replicating vectors.

The disclosed polynucleotide sequences can also be regulated by a second nucleic acid sequence so that the protein or peptide is expressed in a host transformed with the recombinant DNA molecule. For example, expression of a protein or peptide may be controlled by any promoter and/or enhancer element known in the art. Promoters which may be used to control expression include, but are not limited to, the CMV-IE promoter, the SV40 early promoter region (Bernoist and Chambon, (1981) Nature, 290:304-310), the promoter contained in the 3′ long terminal repeat of Rous sarcoma virus (Yamamoto, et al. (1980) Cell, 22:787-797), the herpes simplex thymidine kinase promoter (Wagner, et al. (1981) Proc. Natl. Acad. Sci. U.S.A., 78:1441-1445), the regulatory sequences of the metallothionein gene (Brinster, et al, (1982) Nature, 296:39-42); prokaryotic vectors containing promoters such as the β-lactamase promoter (Villa-Kamaroff, et al. (1978) Proc. Natl. Acad. Sci. U.S.A., 75:3727-3731), or the tac promoter (DeBoer, et al. (1983) Proc. Natl. Acad. Sci. U.S.A., 80:21-25); see also “Useful proteins from recombinant bacteria” in Scientific American (1980) 242:74-94; plant expression vectors comprising the nopaline synthetase promoter region (Herrera-Estrella, et al. (1983) Nature, 303:209-213) or the cauliflower mosaic virus 35S RNA promoter (Gardner, et al. (1981) Nucl. Acids Res., 9:2871), and the promoter of the photosynthetic enzyme ribulose biphosphate carboxylase (Herrera-Estrella, et al. (1984) Nature, 310:115-120); promoter elements from yeast or fungi such as the Gal 4 promoter, the ADC (alcohol dehydrogenase) promoter, PGK (phosphoglycerol kinase) promoter, and/or the alkaline phosphatase promoter.

The vectors according to the invention are, for example, vectors of plasmid or viral origin. In a specific embodiment, a vector is used that comprises a promoter operably linked to a protein or peptide-encoding nucleic acid sequence contained within the disclosed polynucleotide sequences, one or more origins of replication, and, optionally, one or more selectable markers (e.g., an antibiotic resistance gene). Expression vectors comprising polynucleotides of the invention will preferably include at least one selectable marker. Such markers include, for example, dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are well-known in the art.

Expression vectors comprise regulatory sequences that control gene expression, including gene expression in a desired host cell. Preferred vectors for the expression of the polypeptides of the invention include the pET-type plasmid vectors (Promega); pBAD plasmid vectors (Invitrogen); pCR plasmid vectors (Invitrogen); p 75.6 plasmid vectors (Valentis); pCEI plasmid vector (Epimmune); pCEP plasmid vectors (Invitrogen). Vectors for use in bacteria include pHE4-5, pQE70, pQE60 and pQE-9 (QIAGEN, Inc., supra); pBS vectors, Phagescript vectors, Bluescript vectors, pNH8A, pNH16a, pNH18A, pNH46A (Stratagene); and ptrc99a, pKK223-3, pKK233-3, pDR540, pRIT5 (Pharmacia). Among additional eukaryotic vectors are pWLNEO, pSV2CAT, pOG44, pXT1, and pSG (Stratagene); and pSVK3, pBPV, pMSG and pSVL (Pharmacia). Other suitable vectors will be readily apparent to the skilled artisan. Furthermore, the vectors according to the invention are useful for transforming host cells so as to clone or express the polynucleotide sequences of the invention.

The invention also encompasses the host cells transformed by a vector according to the invention. These cells may be obtained by introducing into host cells a nucleotide sequence inserted into a vector as defined above, and then culturing the said cells under conditions allowing the replication and/or the expression of the polynucleotide sequences of the subject invention.

The host cell may be chosen from eukaryotic or prokaryotic systems, such as for example bacterial cells, (Gram negative or Gram positive), yeast cells (for example, Saccharomyces cereviseae or Pichia pastoris), animal cells (such as Chinese hamster ovary (CHO) cells), plant cells, and/or insect cells using baculovirus vectors. In some embodiments, the host cells for expression of the polypeptides include, and are not limited to, those taught in U.S. Pat. Nos. 6,319,691, 6,277,375, 5,643,570, or 5,565,335, each of which is incorporated by reference in its entirety, including all references cited within each respective patent.

Furthermore, a host cell strain may be chosen which modulates the expression of the inserted sequences, or modifies and processes the gene product in the specific fashion desired. Expression from certain promoters can be elevated in the presence of certain inducers; thus, expression of the genetically engineered polypeptide may be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

The subject invention also provides for the expression of a polypeptide, peptide, derivative, or variant encoded by a polynucleotide sequence disclosed herein comprising the culture of an organism transformed with a polynucleotide of the subject invention under conditions that allow for the expression of the peptide, polypeptide, or protein and, optionally, recovering the expressed peptide, polypeptide, or protein.

To improve or alter the characteristics of polypeptides or proteins of the present invention, protein engineering may be employed. Recombinant DNA technology known to those skilled in the art can be used to create novel mutant proteins or muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

For instance, for many proteins, it is known in the art that one or more amino acids may be deleted from the N-terminus or C-terminus without substantial loss of biological function. For instance, Ron and colleagues (J. Biol. Chem., 268:2984-2988 (1993)) reported modified KGF proteins that had heparin binding activity even if 3, 8, or 27 N-terminal amino acid residues were missing. In the present invention, deletions of N-terminal amino acids up to the cysteine at position 6 of SEQ ID NOs:10, 11, 12, 13, and 14 may retain some biological activity such as proliferative erythroid stimulation. Polypeptides having further N-terminal deletions including the cysteine-6 residue in SEQ ID NOs:10, 11, 12, 13, and 14 would not be expected to retain such biological activities because it is known that this residue is likely required for forming a disulfide bridge to provide structural stability which is needed for protein-protein interaction and is in the beginning of the conserved domain required for biological activities.

Accordingly, the present invention further provides polypeptides having one or more residues deleted from the amino terminus of the amino acid sequence of the Modified EPO Constructs 1, 2, 3, 4, and 5, shown in SEQ ID NOs:10, 11, 12, 13, and 14, respectively, up to the cysteine residue at position number 6, and polynucleotides encoding such polypeptides. In particular, the present invention provides polypeptides comprising the amino acid sequence of residues x-193 of SEQ ID NOs:10, 11, 12, 13, and 14, where x is an integer in the range of 1-6, and 6 is the position of the first residue from the N-terminus of the complete Modified EPO Construct 1, 2, 3, 4, and 5 polypeptides (shown in SEQ ID NOs:10, 11, 12, 13, and 14) believed to be required to retain the proliferative erythroid stimulation activity of the wild type EPO.

More in particular, the invention provides polynucleotides encoding polypeptides having the amino acid sequence of residues of 1-193, 2-193, 3-193, 4-193, 5-193, and 6-193 of SEQ ID NOs:10, 11, 12, 13, and 14. Polynucleotides encoding these polypeptides also are provided.

Similarly, many examples of biologically functional C-terminal deletion muteins are known. For instance, Interferon gamma shows up to ten times higher activities by deleting 8-10 amino acid residues from the carboxy terminus of the protein (Dobeli, et al., J. Biotechnology 7:199-216 (1988)). In the present invention, deletions of C-terminal. amino acids up to the cysteine at position 188 of SEQ ID NOs:10, 11, 12, 13, and 14 may retain some biological activity such as proliferative erythroid stimulation. Polypeptides having further C-terminal deletions including the cysteine residue at position 188 of SEQ ID NOs:10, 11, 12, 13, and 14 would not be expected to retain such biological activities because it is known that this residue is likely required for forming a disulfide bridge to provide structural stability which is needed for protein-protein interactions and is the beginning of the conserved domain required for biological activities.

Accordingly, the present invention further provides polypeptides having one or more residues deleted from the carboxy terminus of the amino acid sequence of the Modified EPO Constructs 1, 2, 3, 4, and 5, shown in SEQ ID NOs:10, 11, 12, 13, and 14, up to the cysteine residue at position 188 of SEQ ID NOs:10, 11, 12, 13, and 14, and polynucleotides encoding such polypeptides. In particular, the present invention provides polypeptides having the amino acid sequence of residues 1-y of the amino acid sequence in SEQ ID NOs:10, 11, 12, 13, and 14, where y is any integer in the range of 188 to 193, and residue 188 is the position of the first residue from the C-terminus of the complete Modified EPO Constructs 1, 2, 3, 4, and 5 polypeptides (shown in SEQ ID NOs:10, 11, 12, 13, and 14) believed to be required to retain the proliferative erythroid stimulation activity of the wild type EPO.

More in particular, the invention provides polynucleotides encoding polypeptides having the amino acid sequence of residues 1-188, 1-189, 1-190, 1-191, 1-192, and 1-193 of SEQ ID NOs:10, ii, 12, 13, and 14. Polynucleotides encoding these polypeptides also are provided.

The invention also provides polypeptides having one or more amino acids deleted from both the amino and the carboxyl termini, which may be described generally as having residues x-y of SEQ ID NOs:10, 11, 12, 13, and 14, where x and y are integers as described above.

In addition to terminal deletion forms of the protein discussed above, it also will be recognized by one of ordinary skill in the art that some amino acid sequences of the Modified EPO Constructs 1, 2, 3, 4, and 5 polypeptides can be varied without significant effect of the structure or function of the protein. If such differences in sequence are contemplated, it should be remembered that there will be critical areas on the protein which determine activity.

Thus, the invention further includes variations of the Modified EPO Constructs 1, 2, 3, 4, and 5 polypeptides which show substantial wild type EPO polypeptide activity. Such mutants include deletions, insertions, inversions, repeats, and type substitutions selected according to general rules known in the art so as have little effect on activity. For example, guidance concerning how to make phenotypically silent amino acid substitutions is provided wherein the authors indicate that there are two main approaches for studying the tolerance of an amino acid sequence to change (Bowie, J. U., et al., Science 247:1306-1310 (1990)). The first method relies on the process of evolution, in which mutations are either accepted or rejected by natural selection. The second approach uses genetic engineering to introduce amino acid changes at specific positions of a cloned gene and selections or screens to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described by Bowie and coworkers (supra) and the references cited therein. Typically seen as conservative substitutions, are the replacements, one for another. among the aliphatic amino acids Ala, Val, Leu and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe, Tyr.

Thus, the mutant polypeptide of SEQ ID NOs:10, 11, 12, 13, and/or 14, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the above form of the polypeptide, such as an IgG Fc fusion region peptide or leader or secretory sequence or a sequence which is employed for purification of the above form of the polypeptide or a proprotein sequence. Such fragments, derivatives and analogs are deemed to be within the scope of those skilled in the art from the teachings herein.

Thus, the Modified EPO Constructs 1, 2, 3, 4, and 5 polypeptides of the present invention may include one or more amino acid substitutions, deletions or additions, either from natural mutations or human manipulation. As indicated, changes are preferably of a minor nature, such as conservative amino acid substitutions that do not significantly affect the folding or activity of the protein. Exemplary conservative amino acid substitutions are provided below:

Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Embodiments of the invention are also directed to polypeptides which comprise the amino acid sequence of a Modified EPO Constructs 1, 2, 3, 4, and 5 polypeptide described herein, but further comprising an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions, when compared with the follistatin-3 polynucleotide sequence described herein. Of course, in order of ever-increasing preference, it is highly preferable for a peptide or polypeptide to have an amino acid sequence which comprises the amino acid sequence of a Modified EPO Constructs 1, 2, 3, 4, and 5 polypeptide, which contains at least one, but not more than 10, 9, 8, 7, 6, 5, 4, 3, 2 or 1 conservative amino acid substitutions.

In further specific embodiments, the number of substitutions, additions or deletions in the amino acid sequence Modified EPO Constructs 1, 2, 3, 4, and 5 (SEQ ID NOs:10, 11, 12, 13, and 14, respectively) and/or any of the polypeptide fragments described herein is 75, 70, 60, 50, 40, 35, 30, 25, 20, 15, 10, 9, 8, 7, 6, 5, 4, 3, 2, 1 or 50-75, 25-75, 25-50, 10-25, 1-25, 1-15, 1-10, 1-5,1-4, 1-3 or 1-2.

The invention also encompasses fusion proteins in which the full-length Modified EPO Construct 1, 2, 3, 4 or 5 polypeptides or fragments thereof is fused to an unrelated protein. Such fusion proteins can be routinely designed on the basis of the Modified EPO Constructs 1, 2, 3, 4, and 5 polynucleotide and polypeptide sequences disclosed herein. For example, as one of skill in the art will appreciate, Modified EPO Construct 1, 2, 3, 4, and 5 polypeptides and fragments thereof described herein can be combined with parts of the constant domain of immunoglobulins (IgG), resulting in chimeric (fusion) polypeptides. Such fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker, et al., Nature 331:84-86 (1988)). Fusion proteins that have a disulfide-linked dimeric structure due to the IgG part can also be more efficient in binding and neutralizing other molecules than the monomeric Modified EPO Constructs 1, 2, 3, 4, and 5 polypeptide or polypeptide fragments alone (Fountoulakis, et al., J. Biochem. 270:3958-3964 (1995)). Examples of Modified EPO Construct 1, 2, 3, 4 or 5 fusion proteins that are encompassed by the invention include, but are not limited to, fusion of the Modified EPO Construct 1, 2, 3, 4 or 5 polypeptide sequences to any amino acid sequence that allows the fusion proteins to be displayed on the cell surface (e.g., the IgG Fc domain); or fusions to an enzyme, fluorescent protein, or luminescent protein which provides a marker function.

Variant peptides, polypeptides, and/or proteins of the subject invention can also comprise one or more heterologous polypeptide sequences (e.g., tags that facilitate purification of the peptides, polypeptides, and/or proteins of the invention (see, for example, U.S. Pat. No. 6,342,362, hereby incorporated by reference in its entirety; Altendorf et al. [1999-WWW, 2000] “Structure and Function of the Fₒ Complex of the ATP Synthase from Escherichia Coli,” J. of Experimental Biology, 203:1-9-28, The Co. of Biologists, Ltd., G. B.; Baneyx (1999) “Recombinant Protein Expression in Escherichia coli,” Biotechnology, 10:411-21, Elsevier Science Ltd.; Eihauer, et al. (2001) “The FLAG™ Peptide, a Versatile Fusion Tag for the Purification of Recombinant Proteins,” J. Biochem. Biophys. Methods, 49:455-65; Jones, et al. (1995) J. Chromatography, 707:3-22; Jones, et al. (1995) “Current Trends in Molecular Recognition and Bioseparation,” J. of Chromatography A., 707:3-22, Elsevier Science B. V.; Margolin (2000) “Green Fluorescent Protein as a Reporter for Macromolecular Localization in Bacterial Cells,” Methods 20:62-72, Academic Press; Puig et al. (2001) “The Tandem Affinity Purification (TAP) Method: A General Procedure of Protein Complex Purification,” Methods 24:218-29, Academic Press; Sassenfeld (1990) “Engineering Proteins for Purification,” TibTech 8:88-93; Sheibani (1999) “Prokaryotic Gene Fusion Expression Systems and Their Use in Structural and Functional Studies of Proteins,” Prep. Biochem. & Biotechnol., 29(1):77-90, Marcel Dekker, Inc.; Skerra et al. (1999) “Applications of a Peptide Ligand for Streptavidin: the Strep-tag”, Biomolecular Engineering, 16:79-86, Elsevier Science, B. V.; Smith (1998) “Cookbook for Eukaryotic Protein Expression: Yeast, Insect, and Plant Expression Systems,” The Scientist 12(22):20; Smyth, et al. (2000) “Eukaryotic Expression and Purification of Recombinant Extracellular Matrix Proteins Carrying the Strep II Tag”, Methods in Molecular Biology, 139:49-57; Unger (1997) “Show Me the Money: Prokaryotic Expression Vectors and Purification Systems,” The Scientist, 11(17):20, each of which is hereby incorporated by reference in their entireties), or commercially available tags from vendors such as such as STRATAGENE (La Jolla, Calif.), NOVAGEN (Madison, Wis.), QIAGEN, Inc., (Valencia, Calif.), or InVitrogen (San Diego, Calif.).

The subject invention also concerns novel compositions that can be employed to elicit the production of the ImmunoStealth™ molecules in vivo. In this embodiment of the invention, an amount of a composition comprising recombinant DNA or mRNA encoding an polynucleotide of the subject invention sufficient to induce the production of effective amounts of the ImmunoStealth molecule is administered to an individual. The individual may be monitored for the production of the ImmunoStealth molecule according to methods known in the art (e.g., via serological testing for the molecule using antibody based detection systems known to the skilled artisan).

Methods of introducing DNA vaccines into individuals are well-known to the skilled artisan. For example, DNA can be injected into skeletal muscle or other somatic tissues (e.g., intramuscular injection). Cationic liposomes or biolistic devices, such as a gene gun, can be used to deliver DNA vaccines. Alternatively, iontophoresis and other means for transdermal transmission can be used for the introduction of DNA vaccines into an individual.

Viral vectors for use in the subject invention can have a portion of the viral genome deleted to introduce new genes without destroying infectivity of the virus. The viral vector of the present invention is, typically, a non-pathogenic virus. At the option of the practitioner, the viral vector can be selected so as to infect a specific cell type, such as professional antigen presenting cells (e.g., macrophage or dendritic cells). Alternatively, a viral vector can be selected that is able to infect any cell in the individual. Exemplary viral vectors suitable for use in the present invention include, but are not limited to poxvirus such as vaccinia virus, avipox virus, fowlpox virus, a highly attenuated vaccinia virus, retrovirus, adenovirus, baculovirus and the like. Viral vectors suitable for use in the instant invention are available from commercial vendors, such as Vical, Inc. (San Diego, Calif.) and are used according to the instructions of the manufacturer.

Compositions comprising the subject polynucleotides can include appropriate nucleic acid vaccine vectors (plasmids), which are commercially available (e.g., Vical, San Diego, Calif.) or other nucleic acid vectors (plasmids), which are also commercially available (e.g., Valenti, Burlingame, Calif.). Alternatively, compositions comprising viral vectors and polynucleotides according to the subject invention are provided by the subject invention. In addition, the compositions can include a pharmaceutically acceptable carrier, e.g., saline. The pharmaceutically acceptable carriers are well known in the art and also are commercially available. For example, such acceptable carriers are described in E. W. Martin's Remington's Pharmaceutical Science, Mack Publishing Company, Easton, Pa.

The pharmaceutically acceptable carriers are well known in the art and also are commercially available. For example, such acceptable carriers are described in E. W. Martin's Remington's Pharmaceutical Science, Mack Publishing Company, Easton, Pa. The pharmaceutical compositions comprising ImmunoStealth-modified polynucleotides, peptides, polypeptides, proteins, and/or antibodies of the invention intended for therapeutic or prophylactic treatment are intended for parenteral, topical, oral or local administration. Typically, the pharmaceutical compositions are administered parenterally, e.g., intravenously, subcutaneously, intradermally, or intramuscularly. Because of the ease of administration, the compositions of the invention are particularly suitable for oral administration. Thus, the invention provides compositions for parenteral administration which comprise a solution of the peptides or conjugates dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Compositions comprising peptides polynucleotides, peptides, polypeptides, and/or antibodies of the invention may also be administered via liposomes, which serve to target the compositions to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations, the composition to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide or conjugate of the invention can be directed to the site of lymphoid cells, where the liposomes then deliver the selected polynucleotide, peptide, polypeptide, protein, antibody and/or composition. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369, all of which are incorporated herein by reference.

For targeting to the immune cells, a polynucleotide, peptide, polypeptide, protein, antibody and/or composition to be incorporated into the liposome can include, e.g., antibodies or fragments thereof specific for cell surface determinants of the desired immune system cells. A liposome suspension containing a polynucleotide, peptide, polypeptide, protein, antibody and/or composition may be administered intravenously, locally, topically, etc. in a dose which varies according to, inter alia, the manner of administration, the polynucleotide, peptide, polypeptide, protein, antibody and/or composition being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides, conjugates, and/or conjugates of the invention, and more preferably at a concentration of 25%-75%.

For aerosol administration, the peptides, conjugates, and/or conjugates are preferably supplied in finely divided form along with a surfactant and propellant. Typical percentages of polynucleotide, peptide, polypeptide, protein, antibody and/or composition are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycosides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

The peptides of this invention may also be used to make monoclonal antibodies. Such antibodies may be useful as potential diagnostic or therapeutic agents.

The peptides may also find use as diagnostic reagents. For example, a peptide of the invention may be used to determine the susceptibility of a particular individual to a treatment regimen which employs the peptide or related peptides, and thus may be helpful in modifying an existing treatment protocol or in determining a prognosis for an affected individual. In addition, the peptides may also be used to predict which individuals will be at substantial risk for developing chronic infection.

The polypeptides of the present invention have uses which include, but are not limited to, a molecular weight marker on SDS-PAGE gels or on molecular sieve gel filtration columns using methods well known to those of skill in the art. Additionally, as described in detail herein, the polypeptides of the present invention can also be used as agonists and antagonists capable of enhancing or inhibiting wild type EPO function. Further, such polypeptides can be used in the yeast two-hybrid system to “capture” EPO polypeptide binding proteins which are also candidate agonists and antagonists according to the present invention. The yeast two hybrid system is described by Fields and Song (Nature 340:245-246 (1989)).

The ImmunoStealth process is a stepwise approach based on in silicon sequence analysis methods, combined with high throughput in vitro biochemical evaluations and cellular immunogenicity assays. Single or multiple substitution analogs of a given epitope within the therapeutic protein can also be analyzed by the same methods. The process steps for the identification of Helper T-cell Epitopes are highlighted below:

Specific algorithms are used to predict peptides capable of binding Human Leukocyte Antigens (HLA)

Quantitative biochemical immunogenicity analysis is achieved via in vitro binding assays to 15-17 of the most common HLA molecules

The immunogenicity of an epitope identified above is confirmed by qualitative immunogenicity analysis which is performed using animal or in vitro model systems.

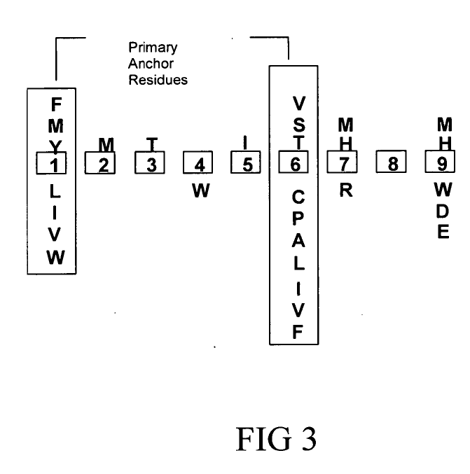

Non-immunogenic analogs of identified epitopes are synthesized and analyzed to ensure reduced or absent immunogenicity and retained biological activity. The steps applied in the ImmunoStealth process are outline schematically in FIG. 3.

The ImmunoStealth process applies our validated Epitope Identification System (EYES™) (Settee, A., et al. (2002) Current Opinion in Investigational Drugs, 3(1):32; Settee, A., et al. (2002) Biologics, 29:271), to map “offending” epitopes. The use of in silicon analysis, a panel of high throughput and quantitative assays coupled with biological and immunological assays and expertise allows the rational selection of epitope analogs. These analogs are then generated and tested for decreased binding and reduced or absent immunogenicity in relevant assay systems. Modified polypeptides, proteins, and/or antibodies consisting of these analogs and then generated to produce a bioactive and less immunogenic drug. This process results in the selection of polypeptide, protein, and/or antibody drugs, including monoclonal antibodies that are clinically safer and more potent therapeutic agents.

Except for the rare case of T-cell independent antigens, development of an antibody response to a protein requires activation of a special type of white blood cell, the Helper T lymphocyte (HTL), which directs specific B lymphocytes to produce antibodies. In general, without activation of HTL, little or no antibody is produced. An HTL becomes activated when the specific T cell receptor recognizes short (10-15 amino acid residues long) peptide sequences (epitopes) bound to specific cellular receptors called HLA Class II. These HLA molecules are expressed by various cell types including macrophages and B cells. Binding of the epitope to the HLA Class II receptor is necessary (but not sufficient) for HTL activation. Thus, peptide binding capacity is directly and highly correlated to the ability of the peptide to elicit an immune response. If no or low binding capacity exists, no HTL activation can occur, and consequently, no antibody response will develop.

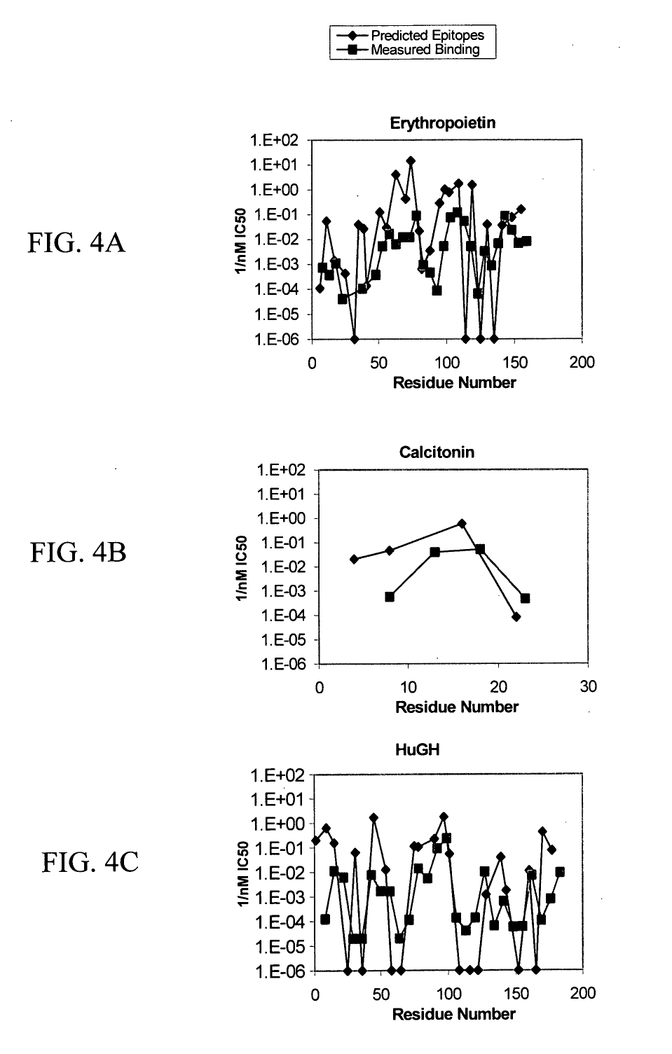

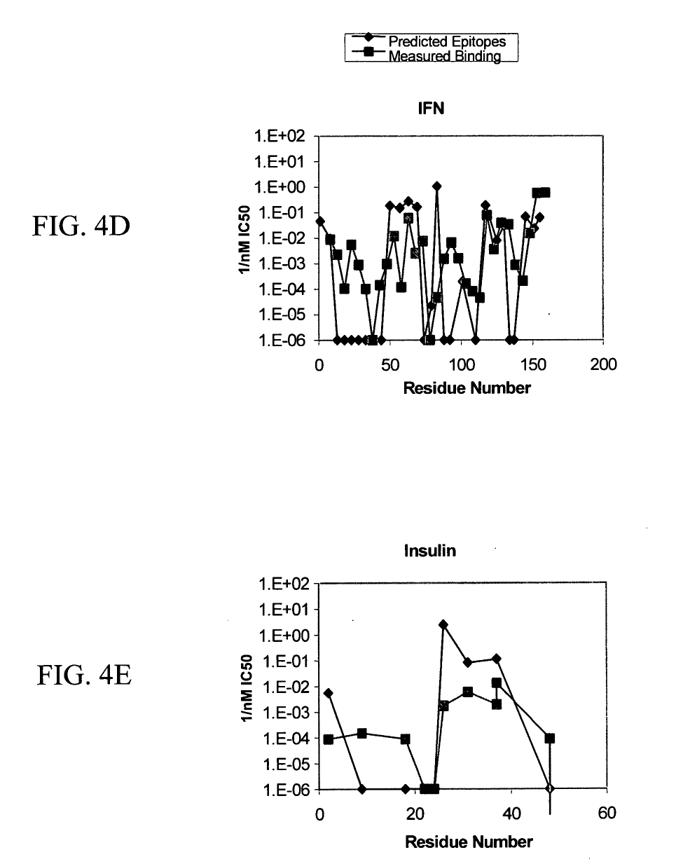

The complex rules for the HLA receptor-peptide interaction have been investigated and deciphered by several groups (Settee, A., et al. (1998) Current Opinion in Immunology, 10(4):478; Ramose, et al. (1993) Annual review of Immunology, 11:213). The peptides binding to a given HLA molecule have distinct sequence characteristics and preferences. For example, amino acids in positions 1 and 6 of the peptide are the primary anchor residues that make direct contact with many HLA molecules (FIG. 4). Some of the other amino acids in the peptide also contribute to the overall binding affinity and are known as secondary anchors.

One element of complexity in the identification of HLA Class II epitopes stems from the fact that HLA molecules are extremely polymorphic. To complicate matters further, different HLA types are found in varying frequencies among different ethnic groups (Manish, T., et al. (1991) Proceedings of the Eleventh International Histocompatibility Workshop and Conference, Tusk, K, et al., editors, Oxford Univ. Press, Tokyo, Japan, p. 1065) as illustrated in Table 9. To globally address identification and removal of unwanted epitopes, the present invention provides a standardized a panel of 17 different assays, each specific for a frequent HLA Class II molecule, which effectively covers the worldwide human population.

As stated above, binding of a peptide to a given HLA molecule is associated with specific sequence characteristics, called motifs or patterns. Motifs can be defined by various methods, such as the sequencing of naturally-occurring legends of a given HLA molecule (Cox, et al. (1994) Science, 264:716; Falk et al. (1991) Nature, 351:290), binding assays performed with synthetic peptides (Settee, A., et al. (1994) 31:813, Fibrin, et al. (1993) 90(4):1508)), and analysis of phage display libraries (Hammer, et al. (1993) Cell, 74(1):197; Hammer, et al. (1992) J. Exp. Med., 176(4): 1007).

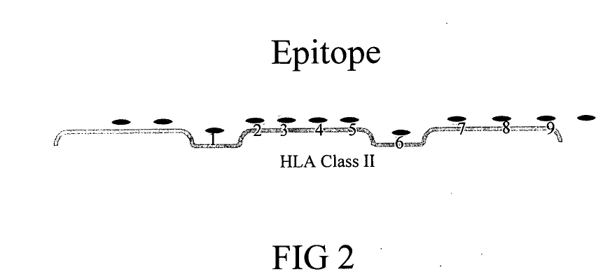

The DR and DQ motifs are relatively well established (Hammer, et al. (1992) J. Exp. Med., 176(4):1007; Sidney, et al. (1992) J. Immunol, 149(8):2634; Calvo-Calle, et al. (1997) J. Immunol., 159(3):1362) and these molecules are associated with the majority of epitopes recognized in humans. An example of a motif associated with a particular DR molecule is shown in FIG. 2. This motif consists of a preference for large aromatic or hydrophobic residues (F, M, Y, L, I, V, W) in position 1 and short and/or hydrophobic residues at position 6 of the peptide legends (SEQ ID NOs:1 and 2). Moreover, preferences for certain amino acids in other non-anchor positions are also seen, for example, M is a preferred amino acid at position 2 in the peptide.

To predict in silicon peptides capable of binding to HLA molecules, various methods have been proposed. These include primary anchor scans (Kubo, et al. (1994) J. Immunol, 152 3913; Pamper, et al. (1991) Nature, 353(6347):792), polynomial functions (Settee, A., et al. (1989) PAS, 86(9):3296; Parker, et al. (1994) J. Immunol., 152(1):163; Hamme, et al. (1994) J. Exp. Med., 180(6):2353), neural nets [Bisset, et al, (1993) J. Mol. Recon., 6(1):41; Glucose, K., et al. (1997) J. Mol. Biol., 267(5):1258) and threading algorithms (Glucose, K., et al. (1997) J. Mol. Biol., 267(5):1258; Alluvia, et al. (1995) J. Mol. Biol. 249(2):244-50). Experiments comparing the success rate of each suggest that the different methods are roughly equivalent (Alluvia, et al. (1995) 249(2):244). A good success rate is usually attained with prediction for one particular HLA allele type. However, to accurately assess a peptide's binding capacity across the entire human patient population up to 10 to 15 different predictions may need to be performed. As a result, even a 90% accuracy for a single allele translates to a meager 25% global accuracy for 13 predictions. Even so, for extremely large molecules, in silicon prediction has proved to be a valid tool to narrow the actual screens with receptor ligand assays.

The most powerful tool to identify and rationally analog (modify) epitopes is the quantitative measurement of HLA-peptide binding affinity. T cell activation can occur only if a peptide binds to the HLA allele above a certain affinity threshold (Keogh, et al., (2001), J. Immunol., 167:787). If a peptide does not bind HLA or binds with an affinity below the threshold, no T cell activation can occur (Keogh, et al. (2001) J. Immunol., 167:787; Settee, A., et al. 1994, J. Immunol., 153:558). The biologically relevant level of binding for HLA molecules has been determined utilizing a number of different experimental approaches (Keogh, et al. (2001) J. Immunol., 167:787; Sette, A., et al. 1994, J. Immunol., 153:558). In humans, most epitopes recognized are bound by HLA-DR molecules (Keogh, et al. (2001) J. Immunol., 167:787; O'Sullivan, et al., 1991, J. Immunol., 147:2663; Hill et al, 1994, J. Immunol., 152(6):2890), even though epitopes bound by HLA DQ and DP molecules have also been described (Baas, et al., (1999) Immunogenetics, 50 (1-2), 8; Falk, K., et al, 1994, Immunogenetics, 39(4):230).

As mentioned above, the issue of HLA polymorphism could potentially complicate the development of an appropriate technology beyond practical feasibility. Indeed, if every different HLA Class II molecule were to recognize a distinct epitope, the number of amino acid changes necessary to introduce in a protein or antibody drug to achieve their obliteration might prove impractical. However, a large fraction of immune reactivity is usually directed against one or a few so-called immunodominant epitopes (van der Most, et al. (1996) J. Immunol., 157(12):5543; Markovic-Plese, et al. (1995) J. Immunol., 155(2):982; Rivoltini, L., et al. (1995) J. Immunol., 154(5)). Moreover, immunodominance is usually associated with high affinity binding to multiple HLA Class II molecules (Panina-Bordignon, et al. (1989) Eur. J. Immunol., 19(12):2237; Rothbard, et al. (1988) Cell, 52(4):515; Diepolder, et al. (1997) J. Virol., 71(8):6011). Table 2 shows representative binding for immunodominant epitopes derived from Tetanus Toxin (“Tet Tox 830”) (SEQ ID NO:15) (Panina-Bordignon, et al. (1989) Eur. J. Immunol., 19(12):2237), Influenza Haemagglutinin (“HA 307”) (SEQ ID NO:16) (Rothbard, et al. (1988) Cell, 52(4):515), Hepatitis C Virus NS3 Protein (“HCV NS3 1242”) (SEQ ID NO:17) (Diepolder, et al. (1997) J. Virol., 71(8):601), Hepatitis B Virus Polymerase Protein (“HBV POL 412”) (SEQ ID NO:18) (Ferrari. C., et al. (1991) J. Clin Invest., 88(1):214) and Plasmodium falciparum (P. fal. SSP2.61”) (SEQ ID NO:19) (Doolan, et al. (2000) J. Immunol., 165(2):1123).

As another example, the influenza hemagglutinin derived epitope HA 307 binds with high affinity to multiple DR molecules (DR1, DR4, DR5, DR6 and DR7). Thus, obliterating the DR binding capacity of this one epitope would be predicted to reduce immune responses against the hemagglutinin protein in patients that express these alleles. Thus, the experimental approach of certain embodiments of the present invention entails the rapid identification of “offending” immunogenic and immunodominant epitopes within a protein of interest by performing binding analysis against HLA molecules representative of the worldwide population. In certain preferred embodiments of the present invention, the strategy utilized to identify potential epitopes depends on the specific protein type analyzed. In the case of chimeric or fully human antibodies, immune reactivity is expected to be focused on the variable regions of the antibody. In the case of proteins in which changes have been introduced at a few specific positions to generate improved molecules, immune reactivity is typically focused on a 20 to 30 residue region centered around the changes (Garrity, et al. (1977) J. Immunol., 165(2):1123; Abrams. et al. (2000) Curr. Opin. Immunol., 12(1):85).

Once epitope sequences capable of binding to multiple HLA Class II molecules are identified, it is desirable to verify that these epitopes are indeed the immunodominant, “offending” epitopes responsible for immunogenicity. In one embodiment, the peripheral blood mononuclear cells (PBMC) of exposed individuals, if available, can be assayed for the presence of activated or memory cells recognizing the epitope (Wilson, et al, 2001, J. Virol., 75(9):4195; Doolan et al. (2000) J. Immunol., 165(2):1123). In an alternative embodiment, primary immunogenicity assays utilizing PBMC from unexposed donors can be utilized (Kawashima, et al. (1999) Cancer Research, 59(2):431). In certain other embodiments, where there is substantial homology between mouse and human molecules, HLA DR transgenic mice (Ito, et al. (1996) J. Exp Med., 183(6):2635; Zeng, et al. (2000) 165:1153) can be used for immunogenicity testing.

Following the epitope identification process (through binding analysis and immunogenicity testing), a number of different analog peptides consisting of single amino acid substitutions or changes are produced and assayed in the panel of binding assays specific for the various HLA DR molecules, representative of the worldwide population. This approach allows specific testing to confirm that the introduced changes lead to reduced or absent binding capacity for multiple molecules. As a preferred embodiment of the present invention, the use of binding assays also prevents the introduction of new HLA reactivities in the analogs generated. For this purpose, systematic testing of the peptides is done against multiple alleles. In preferred embodiments, systematic testing is done against 5, 6, 7, 8, 9, 10, 11, 12, 13, 14, 15, 16, 17, 18, 19 or 20 alleles. In highly preferred embodiments, systematic testing is done against 10, 11, 12, 13, 14 or 15 alleles. In a very highly preferred embodiment, systematic testing is done against 15 alleles. Finally, loss of immunogenicity can also be verified by use of the primary in vitro antigenicity assays or by testing for loss of reactivity against cells from exposed individuals.

The outcome of this series of activities is the definition of a set of analog substitutions recommended for further development in the context of the recombinant protein drug. The bulk of the work to this point is performed in a rapid and inexpensive fashion, taking advantage of assays utilizing short synthetic peptides. It is relevant to emphasize that there is tremendous flexibility in the ImmunoStealth™ technology in that, not only can it be applied to products that are in clinical development and are experiencing problems with immunogenicity; but can also be applied, for example, efficiently during early-stage development to either guide final selection of the lead drug or to custom design the lead drug for low immunogenicity. In certain other embodiments, ImnunoStealth technology can be applied efficiently to any polypeptide, protein, and/or antibody, without regard to its stage of research and development.

Following selection of analogs, full-length versions of the modified polypeptide, protein, and/or antibody engineered for reduced or absent immunogenicity are produced to verify that the substitutions introduced did not alter the biological activity of the polypeptide, protein, and/or antibody. In certain embodiments, in vivo potency assays are used for such verification. In other embodiments, in vitro potency assays are used for such verification. In still other embodiments, pharmokinetic studies are used for such verification. In even still other embodiments, pharmacodynamic studies are used for such verification. In many instances, any of these verification procedures may be combined with, or performed serially, with one or more of the other verification procedures.

The approach of simply modifying known T-cell epitopes has been applied in animal models to produce an anti-epidermal growth factor receptor antibody with reduced immunogenicity (Mateo, et al. (2000) Hybridoma, 19(6), 463). Potential immunogenic epitopes were identified in the variable region of the parent anti-epidermal growth factor receptor antibody and substituted with amino acids that would disrupt such epitopes, the resulting modified antibody was less immunogenic than the parent antibody in monkeys (Mateo, et al. (2000) Hybridoma, 19(6):463). Although these studies resulted in an alteration of immunogenicity by modifying a known HTL epitope in an anti-epidermal growth factor receptor antibody, the studies were not done pursuant to, or as a component of, an established process such as that of the present invention (see supra, Flowchart entitled Exemplary Process Followed For Validation Of ImmunoStealth™ Technology). The efficacy of the approach of simply modifying known T-cell epitopes has been also demonstrated in a mouse model experiment. In this case, a human antibody was modified with respect to murine MHC II alleles. The resulting antibody, which has modified variable regions and murine constant regions, did not elicit an immune response when administered into mice [worldwide website: biovation.co.uk].

Exemplary Process Followed for Validation of Immunostealth™ Technology

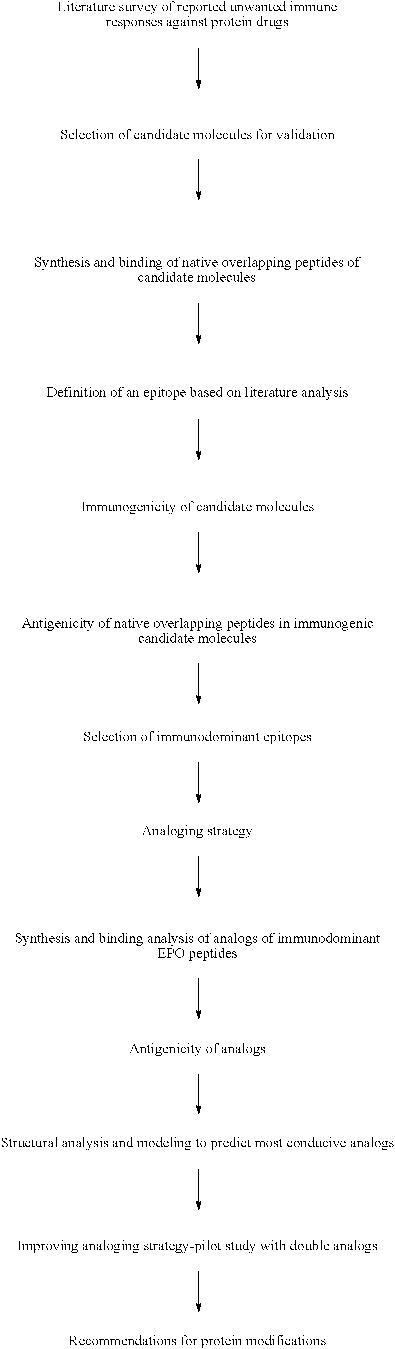

The ImmunoStealth process can be applied to any polypeptide, protein, and/or antibody molecule. Exemplary molecules to which the ImmunoStealth process can be applied include, and are not limited to, IL-1, IL-2, IL-3, IL-4, IL-5, IL-6, IL-7, IL-8, IL-9, IL-10, IL-11, IL-15, Il-16, Il-18, IL-23, IL-24, erythropoietin, G-CSF, M-CSF, platelet derived growth factor (PDGF), MSF, FLT-3 ligand, EGF, fibroblast growth factor (FGF; e.g., aFGF (FGF-1), bFGF (FGF-2), FGF-3, FGF-4, FGF-5, FGF-6, or FGF-7), insulin-like growth factors (e.g., IGF-1, IGF-2); vascular endothelial growth factor (VEGF); interferons (e.g., IFN-γ, IFN-α, IFN-β); leukemia inhibitory factor (LIF); ciliary neurotrophic factor (CNTF); oncostatin M; stem cell factor (SCF); transforming growth factors (e.g., TGF-α, TGF-β1, TGF-β1, TGF-β1), or chemokines (such as, but not limited to, BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP-4, MDC/STCP-1, ABCD-1, MIP-1α, MIP-1β, MIP-2α/GROβ, MP-3α/Exodus/LARC, MIP-30/Exodus-3/ELC, MIP-4/PARC/DC-CK1, PF-4, RANTES, SDF1α, TARC, or TECK). Genes encoding these immunostimulatory molecules are known to those skilled in the art and coding sequences may be obtained from a variety of sources, including various patents databases, publicly available databases (such as the nucleic acid and protein databases found at the National Library of Medicine or the European Molecular Biology Laboratory), the scientific literature, or scientific literature cited in catalogs produced by companies such as Genzyme, Inc., R&D Systems, Inc, or InvivoGen, Inc. (see, for example, the 1995 Cytokine Research Products catalog, Genzyme Diagnostics, Genzyme Corporation, Cambridge Mass.; 2002 or 1995 Catalog of R&D Systems, Inc (Minneapolis, Minn.); or 2002 Catalog of InvivoGen, Inc (San Diego, Calif.) each of which is incorporated by reference in its entirety, including all references cited therein).

Based on a literature survey and personal communications a short list of 5 candidates was generated for the ImmunoStealth™ validation process. The major criteria involved in selection of the candidates was a clear evidence of immunogenicity. The candidates chosen are summarized in Table 3. The choice of the top five candidate molecules was based primarily on clear evidence of immunogenicity as described in the literature and/or by personal communication.

Another criterion is the availability of animal models and/or bioactivity markers for testing functional activity of the protein. The existence of models and markers is a factor because the reduction or elimination of the immunogenic potential of a given molecule must be accomplished without disturbing the functional efficacy of the molecule. Although a structural modeling step is applied to assist in the prediction of non-disruptive changes, reliable methods to determine that protein function is retained are essential.

Several other factors listed in Table 3 present advantages or disadvantages. High homology between the mouse protein sequence and human protein sequence is advantageous because it suggests the possible use of DR transgenic mice to study immunogenicity. Small proteins (30-200 amino acids) are likely to contain fewer epitopes and, therefore, require fewer modifications (i.e., minimal alteration). Proteins that are non-self or have marketed analogs offer a perceptual advantage as candidates for ImmunoStealth-designed sequence modification since these products already contain non-self sequence. The presence of disulfide bonds in the protein adds potential difficulties associated with the synthesis of peptides required for screening and analoging steps. The presence of high molecular weight forms increases the possibility that antibody generation to the protein therapeutic may be related to formulation aggregation issues rather than intrinsic immunogenicity. In this case, removal of linear epitopes will not address the unwanted immunogenicity. Finally, the drug delivery route must be considered. Intravenous delivery is least likely to result in an immune response, whereas, mucosal delivery is perhaps the most immunogenic route. Of the five protein therapeutics summarized below, each rates favorably overall with respect to the criteria considered.

Also included within the scope of the instant application are the disclosures of U.S. patent application Ser. No. 10/103,395, filed Mar. 20, 2002; U.S. patent application Ser. No. 09/009,953, filed Jan. 21, 1998, now U.S. Pat. No. 6,413,517, issued Jul. 2, 2002; and U.S. Provisional Patent Application Nos. 60/036,713, filed Jan. 23, 1997 and 60/037,432 filed Feb. 7, 1997. Each of the patent applications recited in this paragraph are expressly incorporated by reference into the subject specification (including all claims (original, amended, or presented during the course of prosecution), figures, tables, amino acid sequences, nucleic acid sequences, and disclosure). Particularly, Applicants specifically incorporate, by reference, the claims of U.S. patent application Ser. No. 10/103,395, filed Mar. 20, 2003 as well as pages 1-23 of the specification of the '395 patent application.

### EXAMPLES

The following materials and methods were used in the studies presented herein.

Cells. The following Epstein-Barr virus (EBV) transformed homozygous cell lines were used as sources of human HLA Class II molecules: LG2 [DRB1c0101 (DR1)1; GM3107 [DRB50101 (DR2w2a)]; MAT (DRB10301 (DR3)1; PREISS [DRB10401 (DR4w4)1; BIN40 [DRB10404 (DR4w14)1; SWEIG [DRB11101 (DR5w11)]; PITOUT [DRB10701 (DR7)] (a); KT3 [DRB10405 (DR4w15)]; Herluf [DRB11201 (DR5w12)]; HO301 [DRB11302 (DR6w19)]; OLL [DRB10802 (DR8w2)]; and HTC9074 [DRB10901 (DR9), supplied as a kind gift by Dr. Paul Harris, Columbia University]. In some instances, transfected fibroblasts were used: L466.1 [DRB11501 (DR2w2b)]; TR81.19 [DRB30101 (DR52a)]; and L257.6 [DRB40101 (DRw53)]. (Valli, et al. J. Clin. Invest. 91:616 (1993). Cells were maintained in vitro by culture in RPMI 1640 medium supplemented with 2 mM L-glutamine [GIBCO, Grand Island, N.Y.], 50 μM 2-ME, and 10% heat-inactivated FCS [Irvine Scientific, Santa Ana, Calif.]. Cells were also supplemented with 100 μg/ml of streptomycin and 100 U/ml of penicillin [Irvine Scientific]. Large quantities of cells were grown in spinner cultures.

Cells were lysed at a concentration of 10⁸ cells/ml in PBS containing 1% NP-40 [Fluka Biochemika, Buchs, Switzerland], 1 mM PMSF [CalBioChem, La Jolla, Calif.], 5 mM Na-orthovanadate, and 25 mM iodoacetamide [Sigma Chemical, St. Louis, Mo]. The lysates were cleared of debris and nuclei by centrifugation at 10,000×g for 20 min.

Affinity purification of HLA-DR molecules. Class II molecules were purified by affinity chromatography as previously described (Sette, et al. J. Immunol 142:35 (1989) and Gorga, et al. J. Biol. Chem. 262:16087 (1987)) using the mAb LB3.1 coupled to Sepharose 4B beads. Lysates were filtered through 0.8 and 0.4 μM filters and then passed over the anti-DR column, which were then washed with 15-column volumes of 10 mM TRIS in 1% NP-40, PBS and 2-column volumes of PBS containing 0.4% n-octylglucoside. Finally, the DR was eluted with 5 mM diethylamine in 0.15M NaCl containing 0.4% n-octylglucoside, pH 11.5. A 1/25 volume of 2.0M Tris, pH 6.8, was added to the eluate to reduce the pH to 8.0, and then concentrated by centrifugation in Centriprep 30 concentrators at 2000 rpm (Amicon, Beverly, Mass.).

Class II peptide-binding assays. A panel of 13 different specific DR-peptide assays were utilized in the present study. These assays were chosen as to be representative of the most common DR alleles. Table 1 lists for each DR antigen, the representative allelic product utilized, the cell line utilized as a source of DR, and the radiolabled probe utilized in the assay. Purified human Class II molecules [5 to 500 nM] were incubated with various unlabeled peptide inhibitors and 1-10 nM ¹²⁵I-radiolabeled probe peptides for 48 h in PBS containing 5% DMSO in the presence of a protease inhibitor cocktail. The radiolabeled probes used were HA Y307-319 (SEQ ID NO: 16) (DR1), Tetanus Toxoid[TT] 830-843 (SEQ ID NO: 15) (DR2w2a, DRSw111, DR7, DR8w2, DR8w3, DR9), MBP 78-101 (SEQ ID NO: 253) (DR2w2b), TT1272-1284 (SEQ ID NO: 138) (DR52a), MT 65 kD Y3-13(SEQ ID NO: 148) with Y7 substituted with F (SEQ ID NO: 249) for DR3, a non-natural peptide with the sequence YARFQSQTTLKQKT (SEQ ID NO: 250) (DR4w4, DR4w15, DRw53) (Valli, et al. supra), and for DR5w12, a naturally processed peptide eluted from the cell line C1R, EALIHQLINPYVLS (SEQ ID NO:251) (DR5w12) and 650.22 peptide, (TT 830-843 A→S836 analog), (SEQ ID NO: 252) for DR6w19.

Radiolabeled peptides were iodinated using the chloramine-T method. Peptide inhibitors were typically tested at concentrations ranging from 1201 μg/ml to 1.2 ng/ml. The data were then plotted and the dose yielding 50% inhibition (IC50) was measured. In appropriate stoichiometric conditions, the IC50 of an unlabeled test peptide to the purified DR is a reasonable approximation of the affinity of interaction (Kd). Peptides were tested in two to four completely independent experiments. The final concentrations of protease inhibitors were: 1 mM PMSF, 1.3 nM 1.10 phenanthroline, 73 μM pepstatin A, 8 mM EDTA, and 200 μM N alpha-p-tosyl-L-lysine chloromethyl ketone (TLCK) [All protease inhibitors from CalBioChem, La Jolla, Calif.]. Final detergent concentration in the incubation mixture was 0.05% Nonidet P-40. Assays were performed at pH 7.0 with the exception of DR3, which was performed at pH 4.5, and DRw53, which was performed at pH 5.0. The pH was adjusted as previously described (Sette, et al. (1992) J. Immunol., 148:844).

Class II peptide complexes were separated from free peptide by gel filtration on TSK2000 columns (TosoHaas 16215, Montgomeryville, Pa.), and the fraction of bound peptide calculated as previously described (Sette, et al., (1989) supra). In preliminary experiments, the DR preparation was titered in the presence of fixed amounts of radiolabeled peptides to determine the concentration of Class II molecules necessary to bind 10-20% of the total radioactivity. All subsequent inhibition and direct binding assays were the performed using these Class II concentrations.

DRB1 specificity of DR4w15, DR6w19, DR8w2, DR8w3, and DR9 assays.

Because the antibody used for purification is α-chain specific, β1 molecules are not separated from β3 (and/or β4 and β5) molecules. Development and validation of assays in regard with DRβ chain specificity has been described in detail elsewhere for many of the DR alleles listed above (108). Herein we describe for the first time DR4w15, DR6w19, DR8w2, DR8w3, and DR9 assays. Experiments addressing the β chain specificity of these new assays are described in the present section.

DR4w15. The β4 product DRw53 is co-expressed with DR4w15 and the determination of the specificity of the DR4w15 binding assay is complicated in that the same radiolabeled ligand is used for both the DR4w15 and DRw53 binding assays. Since typically β1 chains are expressed at 5-10 fold higher levels than other β chains, and all binding assays are performed utilizing limiting DR amounts, it would be predicted that the dominant specificity detected in the assay would be DR4w15. To verify that this was indeed the case, the binding pattern of a panel of 58 different synthetic peptides in the putative DR4w15 specific assay with that obtained in a DRw53 specific assay (which uses a DRw53 fibroblast as the source of Class II molecules). Two very distinct binding patterns were noted, and in several instances, a peptide bound to one DR molecule with high affinity, and did not bind to the other (data not shown).

DR6w19. The DR6w19 assay utilizes as the source of Class II molecules the EBV transformed homozygous cell line H0301, which co-expresses DRB30301 (DR52a). While the radiolabeled ligand used in the DR6w19 assay is different than that used for the DR52a assay, the ligand is related (i.e., is a single substitution analog) to a high affinity DR52a binder. As was done in the case of DR4w15, the specificity of the assay was investigated by analyzing the binding capacity of a panel of naturally occurring peptides for DR6w19 and DR52a. The two assays demonstrated completely different binding specificities. For example, in terms of relative binding, TT 1272-1284 binds 63-fold better in the DR52a assay than in the DR6w19 assay. Conversely, the invariant chain peptide binds 189-fold better in the DR6w19 assay. In conclusion, these data demonstrated that the binding of the radiolabeled peptide 650.22 to purified Class II MHC from the H0301 cell line is specific for DR6w19.

DR8w2 and DR8w3. The β1 specificity of the DR8w2 and DR8w3 assays is obvious in that no β3 (and/or B4 and β35) molecule is expressed.

DR9. The specificity of DR9 assay is inferred from previous studies which have shown that the TT 830-843 radiolabeled probe peptide does not bind to DRw53 molecules (Alexander, et al., (1994) Immunity, 1:751).

### Example 1

### ImmunoStealth™ Analysis of Five Therapeutic Proteins

Amino acid sequences (peptides) which are capable of binding multiple HLA MHC molecules are described as degenerate. Given the large degree of HLA polymorphism, identifying epitopes which are capable of binding more than one MHC specificity is crucial to the development of effective peptide-based vaccines having the capacity to cover a large fraction of the general population without ethnic bias. Alternatively, identifying degenerate epitopes provides a route to modify therapeutic proteins that elicit unwanted T-cell responses. For example, changing “analoging” the amino acid composition of a few degenerate epitopes within a therapeutic protein might disrupt binding to MHC class II molecules, resulting in a reduction, or even elimination, of unwanted T-helper responses towards the protein. If the changes are minimal and judiciously made, the overall structure and function of the native therapeutic protein can be maintained. As a result, the modified protein maybe less immunogenic, and therefore more potent and efficacious.

As described herein, certain embodiments of the present invention are useful as a method to identify within protein sequences, regions that are immunogenic in a large fraction of the individuals of the general population. This method incorporates bioinformatic analyses that predict peptides with degenerate MHC binding capacity, with MHC-peptide binding assays and cellular assays that directly measure degeneracy and immunogenicity. Further embodiments of the present invention are also useful to: (a) determine efficacy of the algorithms for predicting binding and degeneracy; (b) compare the algorithm with others; and (c) analog degenerate regions of particular polypeptides, proteins, and/or antibodies to reduce binding to class II MHC molecules.

PIC Algorithm

The PIC algorithm is an embodiment of the present invention. PIC is a modified linear coefficient, or matrix, based method for predicting peptides with MHC binding capacity. Like other matrix-based methods, PIC operates on the assumption that each residue along a peptide molecule can independently contribute to binding affinity (see Sette, A., et al. (1989) Proc. Natl. Acad. Sci. U.S.A., 86:3296-3300. Like other matrix-based algorithms developed (see, Gulukota, K., et al. (1997) J. Mol. Biol., 267:1258-67), PIC generates a score for individual peptides that is derived from polynomial coefficients describing the relative binding associated with each of the 20 naturally occurring amino acid residues for each peptide position. Next, certain mathematical transformations are performed, including linear polynomial scaling, an experimental power transformation, and a further linear correction base on minimizing the deviation of predicted values from experimental values. Based on these operations the algorithm yields a predicted IC50 value (designated as “PIC”) for the corresponding input sequence. Because PIC converts coefficient-based scores into an IC₅₀ prediction, it allows for searches which include different peptide sizes or alleles. Lower PIC values indicate a higher probability of binding to MHC. As PIC is the only algorithm tested in which lower values indicate better binding, the output data of the algorithm was normalized to other algorithms by performing 1/PIC calculations.

SYFPEITHI Algorithm

The SYFPETIHI algorithm (Rammensee, Bachmann & Stevanovic (1997) MHC ligands and peptide motifs (Landes Bioscience, Heidelberg)), another matrix-based method, provides predictions based on the abundance of amino acids in specific positions in T cell epitopes, natural ligands or binding peptides, and (Rammensee, H. G., et al., (1995) Immunogenetics 41:178-228) incorporates published motifs (pool sequencing, natural ligands). The algorithm takes into consideration the amino acids in the anchor and auxiliary anchor positions, as well as other frequent amino acids. The score is calculated according to the following rules: the amino acids of a certain peptide are given a specific value depending on whether they are anchor, auxiliary anchor or preferred residue. Ideal anchors are given 10 points, unusual anchors 6-8 points, auxiliary anchors 4-6 and preferred residues 1-4 points. Amino acids that are regarded as having a negative effect on the binding ability are given negative values between −1 and −3. The scoring system evaluates every amino acid within a given peptide. The allocation of values is based on the frequency of the respective amino acid in natural ligands, T-cell epitopes, or binding peptides (Rammensee, H., et al., (1999) Immunogenetics, 50:213-219).

Probed Algorithm

The Propred algorithm (Singh, H. and Raghava, G. P. (2001) Bioinformatics. 17, 1236-37), uses quantitative matrices based on the method published by Sturniolo, et al. (Sturniolo, T., et al. (1999) Nat. Biotechnol., 17:555-561) to derive scores for nonamers peptides. The score represents the probability of a peptide to bind to MHC, with a higher score indicating a greater binding probability. The value predicted for each nonamer is based on the data set for that particular class II molecule. The algorithm can predict peptides with promiscuous binding to multiple HLA Class II molecules. The predicted binders can be visualized either as peaks in graphical interface or as colored residues in HTML interface.

MHC Thread Algorithm

The MHC Thread algorithm (Altuvia, Y., et al., (1997) Hum. Immunol,. 58:1-11) is designed to predict 15-mer peptides which are likely to bind to class II MHC molecules by assigning a binding score for all possible overlapping 15-mer peptides in a submitted protein sequence. The binding score is derived by assessing potential three-dimensional conformations of each residue of a peptide within the MHC binding pockets. A number of factors in the interaction are considered, including the number of peptide residues that fit within the respective MHC binding groove, steric overlap between peptide and MHC residues, the number of hydrogen bonds that can be formed, the strength of electrostatic interactions between any polar atoms, and the number of favorable contacts. The number and nature of hydrophobic and hydrophilic interactions between peptide and MHC residues are also evaluated. Peptides with higher scores are predicted to be more likely to bind to class II MHC molecules.

Peptide Synthesis and Radiolabeling

Peptides were synthesized at Epimmune (San Diego, Calif.) as described elsewhere (Dzuris, J. L., et al., (2000) J. Immunol. 164:283-91) or were purchased as crude material from Mimotopes (Minneapolis, Minn.) or Pepscan (Lilystad, The Netherlands). Peptides synthesized at Epimmune were typically purified to >95% homogeneity by reverse-phase HPLC. Purity of Epimmune-synthesized peptides was determined using analytical reverse-phase HPLC and amino acid analysis, sequencing, and/or mass spectrometry. Lyophilized peptides were resuspended at 4-20 mg/ml in 100% DMSO and then diluted to required concentrations in PBS 0.05% (v/v) Nonidet P-40 (Fluka Biochemika, Buchs, Switzerland). Peptides were ¹²⁵I-radiolabeled using chloramine T methodology, as previously described (Sidney, J., et al., (1998) Curr. Prot. Immunol., 18.3.1-18.3.19).

For the present study, peptides were derived from the amino acid sequences of salmon calcitonin (amino acids 83-114 of P01263) (SEQ ID NO:4), human erythropoietin (amino acids 28-193 of P01588) (SEQ ID NO:3), human growth hormone 1 isoform 1 (amino acids 27-217 of P01241) (SEQ ID NO:5), human insulin alpha (amino acids 90-110 of P01308) (SEQ ID NO:6), human insulin beta (amino acids 25-54 of P01308) (SEQ ID NO:7), and human interferon beta (amino acids 22-187 of AAC41702) (SEQ ID NO:8) [see FIG. 11].

Peptide-Binding Assays

Quantitative assays to measure the binding of peptides to solubilized class II molecules are based on the inhibition of binding of a radiolabeled standard peptide. Binding assays were performed as described previously (Sidney, J., et al. (1998) Curr. Prot. Immunol., 18.3.1-18.3.19). Briefly, 1-10 nM radiolabeled peptide was coincubated for 2 days at either room temperature or 37 degrees Celsius with 1 μM to 1 nM purified MHC in the presence of a cocktail of protease inhibitors. Assays were performed at various pH conditions, ranging from pH=4 to pH=7. The final pH of assay mixtures was adjusted using citrate buffer as described elsewhere (Sidney, J., et al. (1998) Curr. Prot. Immunol., 18.3.1-18.3.19).

After incubation, the percentage of MHC-bound radioactivity was determined by size exclusion gel filtration chromatography using a TSK 2000 column (Toso Haas, Montgomeryville, Pa.). Alternatively, the percentage of HLA-DR-bound radioactivity was determined by capturing MHC/peptide complexes on Optiplates (Packard Instrument, Meriden, Conn.) coated with the LB3.1 mAb (Pan-DR) and determining bound cpm using the TopCount microscintillation counter (Packard Instrument).

In preliminary assays, candidate labels were tested for direct binding to purified MHC molecules. The amount of MHC yielding 10-20% bound radioactivity was used in subsequent inhibition of binding assays in which the concentration of peptide yielding 50% inhibition of the binding of the radiolabeled peptide was calculated. Under the conditions used, in which [label]<MHC and IC50>[MHC], the measured IC50 values are reasonable approximations of the true K_{d} values. Each competitor peptide was tested in two to four independent experiments. As a positive control, the unlabeled version of the radiolabeled probe was also tested in each experiment. Peptides were initially tested at one or two high doses. The IC50 of peptides yielding positive inhibition were then determined in subsequent experiments, in which two to six further dilutions were tested. As in previous studies, peptides with an affinity for a specific class II molecule of 1000 nM or better are defined as binders for the respective molecules (Sidney, J., et al. (1998) Curr. Prot. Immunol., 18.3.1-18.3.19).

Donors

Leukopheresis units from healthy donors were obtained through the General clinical research center at Scripps Clinic, San Diego Calif. PBMC were purified using density gradient sedimentation and Ficoll-Paque (Pharmacia, Upsala, Sweden) and frozen at a density of approximately 50×10⁶ cells/vial

MHC Typing

DNA was prepared from PBMC using QIAGEN DNA purification reagents. Typings for HLA Class II genes (DRB1/3/4/5, DQB1) were performed using sequence based typing of exon 2. For each gene, this involved a locus specific PCR amplification of exon 2. This provided template for sequencing the exon in both orientations using custom primers and DYEnamic ET Terminator (Amersham Biosciences) chemistry on an ABI 377 DNA Sequencer. The resulting data was then analyzed on MatchTools HLA typing software (ABI) to generate a final type.

Immunogenicity of Candidate Proteins

a) Generation of DC

GM-CSF and IL-4 induced dendritic cells were generated from these PBMC using standard protocols (Keogh, et al. (2001) J. Immunol, 167:787-796). The dendritic cells were harvested on day 6 and matured using 75 ng/ml of recombinant human TNFα. The following day, the DC were collected and pulsed for 4 hours with recombinant EPO protein (R and D systems) or human growth hormone (Serono laboratories) at a cell concentration of 1×10⁶/ml containing 10% AB serum. The DC were then washed extensively, irradiated (4200 Rads) and used for induction.

Induction of HTLs with DC. CD4+ T cells were isolated by selection using antibody-coated magnetic beads (Miltenyi Biotec, Auburn, Calif.) according to manufacturer's instructions. Typically 400-500×10⁶ PBMCs were processed to obtain 50×10⁶ CD4 cells enough for a 48-well plate. A total of 1×10⁶ CD4 was co-cultured with 5×10⁴ DC in each well of a 48-well plate. Cultures were routinely fed with 10 U/ml of IL-2 and IL-12 every 3 days. On day 14 following induction, the cultures were re-stimulated with protein pulsed PBMCs. Briefly, PBMCs (2×10⁶/ml) were pulsed with 10 μg/ml of peptide or whole antigen for 4 hours at 37° C. PBMC were then washed, irradiated, and 1×10⁶ of these antigen pulsed PBMCs were then added to each well of the 48-well plate. After two re-stimulations an ELISPOT assay was performed to detect peptide-specific responses. The T cell lines generating a positive signal by ELISPOT (described below) were frozen and expanded in vitro with additional in vitro re-stimulations with antigen-pulsed PBMCs.

ELISPOT Assay: 96 well membrane-backed ELISA plates (Millipore) were coated with anti-human IFN-γ monoclonal antibody (Mabtech) overnight at 4° C., and then blocked for 1 hour at 37° C. with media containing 10% AB serum. Effector T cells were isolated from the induced cultures (testing was done typically 10-14 days after the last stimulation), washed and resuspended at 2×10⁶/ml. Several different dilutions of these effector T cells in 50 μl volume were plated onto coated ELISPOT plates, and cultured with 10⁵ irradiated, antigen-pulsed PBMC for 20 hours at 37° C. with 5% CO₂. The following day, the cells were washed out, and the number of IFN-γ secreting cells are detected by incubation with biotinylated anti human IFN-γ antibody (Mabtech), followed by incubation with Avidin-Peroxidase Complex (Vectastain). Finally, the plates are developed using AEC (3-amino-9-ethyl-carbazole; Sigma), washed and dried. Spots are counted utilizing the Zeiss KS ELISPOT reader. A positive response is defined as a p value (one-tailed t test) of ≦0.05 calculated between the irrelevant and relevant antigen wells. Data is presented as net spots/10⁶, calculated as (mean number of spots against relevant antigen-mean number of spots against irrelevant antigen)×dilution factor of effector cells. Therefore if effector cell concentration used was 5×10⁴/well, the dilution factor was 20 to reflect the number of spots/10⁶.

Antigenicity Analysis

One week before the antigenicity analysis, T cell lines identified above were tested for protein specific responses in an ELISPOT assay to confirm specificity and to determine optimal effector cell concentrations to use for the antigenicity assay. The day before the assay, ELISPOT plates were coated as described above. PBMC from autologous donors were thawed and pulsed with relevant and irrelevant protein for 4 hours, after which the cells were washed, irradiated and used as APC in the ELISPOT assay. In the case of peptide pulsing, PBMC were irradiated first, and then pulsed with 10 μg/ml of appropriate relevant and irrelevant peptides. Peptide pulsing was done in 96 well round bottom plates to enable ease of washing and further plating on to coated ELISPOT plates. Following pulsing for 3 hours at 37° C., the cells were washed with PBS and used as antigen presenting cells. 50 μl of effector cells (at concentrations determined above) were incubated with 50 μl of peptide or protein pulsed PBMC (10⁵/well) for 24 hours and developed the following day. Data analysis was carried out against irrelevant antigen or relevant antigen pulsed targets for all individual peptides. Each experiment was performed twice and a third time if there was discordant data.

Analoging Method

To judiciously change specific amino acid residues in epitopes, each 9-mer core for the most degenerate epitopes were considered. These epitopes comprised the EPO 101, Insulin 12, EPO 136 and Calcitonin 11 regions. Each of these 15-mer peptides were associated with 15 different core regions (9-mer cores). Within each core, there were 20 possible amino acids×9 positions, leading to 180 possible substitutions. For each epitope, there were a total of 2700 different possible analogs (15 different core regions×18 analogs).

To reduce the amount of possible analogs, particular types of substitutions were recommended. P1 anchors are most frequently F, W, Y, L, I, V or M. Cysteine was avoided due to possible disulfide bond formation. The remaining 12 amino acid residues were used for substitution analysis. The PIC algorithm identified D, R, and G residues as those which most frequently result in deletion of a proposed PIC binder.

Given these considerations, the substitution strategy entailed identifying the core regions using PIC algorithm analysis. For each of the main cores, all 12 amino acids were used to make substitutions at the P1 position. For each of the secondary cores, all 12 amino acids were used to make substitutions at the P1 position. D, R, and G residues were used at each of the remaining positions of the main core. This resulted in the generation of 48 analogs for each target degenerate epitope. These analogs were then tested for MHC binding to determine their reduction in MHC affinity.

Restriction Mapping

Single stable transfectants of fibroblast cells carrying either the DRB1*0301 or DRB4*0101 molecules were thawed and checked for expression of DR molecules. An EBV transformed line expressing both DRB1*0901 and DRB4*0101 was also thawed and checked for expression. For restriction mapping experiments, these fibroblast or EBV cell lines were used as peptide antigen presenting cells instead of the PBMC described above. The protocol for peptide pulsing and the ELSIPOT was identical except that a fewer number of peptide pulsed APC (10⁴ cells/well) were used instead of 10⁵ PBMC/well during the ELISPOT assay. Appropriate controls using both untransfected parent lines and irrelevant peptide pulsed transfectants were used. Data was calculated and presented as described above.

Structural Analysis

a) Analysis of Evolutionary Conservation of Variation within Target Peptides

The primary amino acid sequences for erythropoietin were aligned used BLASTP 2.2.3 (Altschul, (1997), Nucl. Acids Res., 25:3389-3402). Human erythropoietin (P01588) (SEQ ID NO:9) was used as the query sequence. Twenty-four mammalian erythropoietin molecules were aligned and analyzed. These included sequences representing human, macaque, cat, dog, cow, pig, sheep, rabbit, mouse, and rat erythropoietin proteins.

In order to determine structural conservation of residues, the structures of erythropoietin's structural neighbors (erythropoietin (1EER:A, 1CN4:C, 1BUY:A), ciliary neurotrophic factor (1CNT3), interleukin-6 (1ALU), stem cell factor (1EXZ:A), prolactin (1F6F:A), growth hormone (1HUW), granulocyte colony-stimulating factor (1BGC), macrophage colony-stimulating factor (1HMC:B), leukemia inhibitory factor (1LKI), interleukin-4 (1RCB), oncostatin (1EVS:A), and granulocyte-macrophage colony-stimulating factor (2GMF:A) were aligned using FSSP (Holm, L. and Sander, C. (1996) Science, 273(5275):595-60) and analyzed.

b) Generate and Analyze Structural Models for Erythropoietin Variants

Three erythropoietin structures (1EER:A, 1CN4:C, and 1BUY:A) were evaluated as potential templates for variant model building. The 1EER:A structure was chosen to serve as the template for modeling the erythropoietin variants due to its high resolution and low R values especially in the erythropoietin protein domains of interest.

Both published and experimental variants were modeled using the erythropoietin structure 1EER:A as the template, Swiss-Model for model building and Deep View and WHATCHECK for subsequent analysis (Guex, N. and Peitsch, M. C. (1997) SWISS-MODEL and the Swiss-PdbViewer: An environment for comparative protein modelling. Electrophoresis 18:2714-2723. Peitsch, M. C. (1996); ProMod and Swiss-Model: Internet-based tools for automated comparative protein modelling. Biochem Soc Trans 24:274-279; Peitsch, M. C. (1995); Protein modeling by E-mail Bio/Technology 13:658-660, WHAT IF: A molecular modeling and drug design program. G. Vriend, J. Mol. Graph. (1990) 8:52-56). See also, Hooft, R. W. W., et al, (1996) Nature 381:272.

To assess the accuracy of the PIC algorithm for predicting binding, we synthesized overlapping 15mers spanning entire sequences of salmon calcitonin (amino acids 83-114 of P01263) (SEQ ID NO: 4), human erythropoietin (amino acids 28-193 of P01588) (SEQ ID NO: 3), human growth hormone 1 isoform 1 (amino acids 27-217 of P01241), human insulin alpha (amino acids 90-110 of P01308) (SEQ ID NO: 6), human insulin beta (amino acids 25-54 of P01308) (SEQ ID NO: 7), and human interferon beta (amino acids 22-187 of AAC41702) (SEQ ID NO: 8). These peptides were tested for binding to DRB1*0101, as described in the materials and methods section. A peptide was considered a binder if it was associated with a minimal affinity of 1000 nM IC50 (Southwood, S., et al., (1998) J. Immunol,. 160, 3363-73). The binding data was normalized by performing a 1/IC50 value calculation, similar to the PIC calculation.

In FIG. 4, predicted DRB1*0101 binding is plotted with the measured DRB1*0101 binding data. Using a threshold of 1/1000 IC50 value for binding (Southwood, S., et al. (1998) J. Immunol., 160:3363-73), we found sixteen binding regions as defined by at least two sequential peptides which were shown to bind with high affinity. Given that the peptides were designed to overlap and therefore share residues, two sequential binders confirm a high affinity region. 11 of the 16 (67%) binding regions from all the molecules were accurately predicted by PIC. On the other hand, PIC predicted that 13 regions would have been associated with high DRB1*0101 binding affinity. As shown, 11 (85%) of these were indeed high affinity binding regions. Thus, these data demonstrate that PIC is sensitive, identifying 67% of the DRB1*0101 binding regions, but also has a relatively high positive predictive value in that 85% of the regions identified by PIC were associated with DRB1*0101 binding.

Comparison of PIC with Other Popular Algorithms

The abilities of additional algorithms to predict binding to DRB1*0101 were then evaluated. The additional algorithms examined were Propred, SYFPEITHI and MHC Thread, as these are publicly available predictive algorithms for MHC binding. To allow direct comparison of different algorithms, measures of sensitivity (SENS) and positive predictive values (PPV; or efficiency when the same data sets are utilized) are commonly utilized. Sensitivity is the fraction of all possible positives that are identified by an algorithm. PPV measures the number of positives identified by the algorithm that are truly positive (i.e., correct predictions). Because it is not always desirable, or possible, to synthesize large sets of peptides, it is often practical to allow lower sensitivity in prediction, while demanding higher efficiency. In this scenario, one can synthesize fewer peptides, but be more assured that the peptides selected will be positive hits. Thus, in the analysis below, we have a compared the efficiency of the different algorithms at various levels of sensitivity.

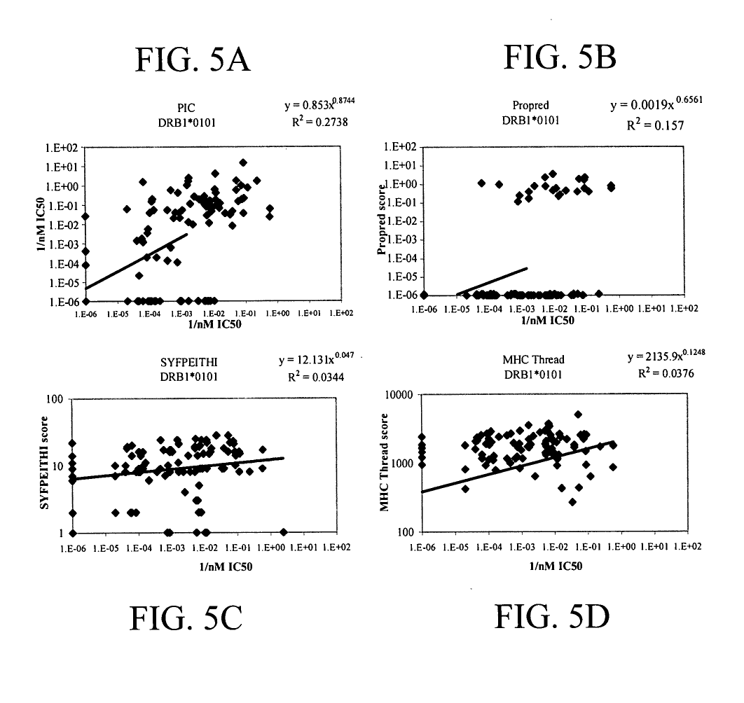

In FIG. 5, a regression analysis is presented that shows the output of each algorithm versus the measured binding to DRB1*0101. The measured binding data is shown on the x-axis, while the predicted binding data is shown on the y-axis. Each peptide for which we have binding data was analyzed by each predictive algorithm. The results are shown in this regression analysis for each predictive algorithm. Table 4 contains additional analyses that summarize the efficiency of each algorithm as a function of sensitivity. As shown, when all of the binders were to be identified (SENS=100) the algorithms performed similarly, with each having an efficiency of about 50%. However, when a sensitivity of 75%, or lower, was allowed PIC clearly performed better than the other algorithms. Specifically, PIC maintained a level of efficiency in the 78-84% range, when SENS was between 34% and 75%. MHC Thread and SYFPEITHI had efficiencies in the 51-75% range, depending on the algorithm and SENS level considered. Propred had the most varied performance, with remarkably low efficiencies (25-37% range) at the 50 and 75% SENS levels, but a commendable 82% efficiency at the lowest SENS level.

In conclusion, these data have demonstrated that the PIC algorithm is overall the most efficient algorithm. It offers comparable or optimal performance throughout all ranges of evaluation. However, even PIC performs at only 51% efficiency when detecting all the binding peptides (see Table 4). Furthermore, the algorithm only predicts binding to DRB1*0101. The MHC loci are extremely polymorphic. A more feasible approach may be to measure binding to the most prevalent MHC molecules and assessing the extent of binding promiscuity (or degeneracy).

Identification of Degenerate MHC Binding Regions

To accurately identify degenerate regions of the target proteins analyzed herein, the studies were focused on additional MHC class II molecules. The ability of overlapping peptides from each of the proteins to bind multiple MHC class II molecules was characterized using our panel of 15 MHC class II binding assays (Tables 5A-E). This panel of DR specificities was assembled to include DR molecules expressed at high frequency in the general population.

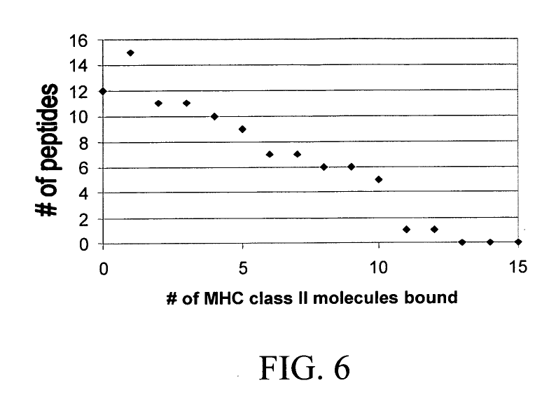

Interestingly, most of the peptides bound one or more MHC class II molecules. FIG. 6, shows that only 12% of the peptides tested did not bind to any of the molecules tested. The range of molecules bound by a peptide varied from 1 to 12 molecules. These data suggest that to generate proteins that are completely devoid of immunogenicity may be unrealistic. A more practical alternative is to identify epitopes that bind to multiple HLA class II molecules, thereby focusing on those epitopes that may be a problem in a significant proportion of the worldwide population. Targeting of such epitopes would therefore, reduce the immunogenicity of the protein to a significant extent, though not eliminate it completely, as epitopes that bind to 1-2 specific MHC molecules will likely always be present. To determine the validity of this approach, an analysis of known immunodominant class II epitopes was carried out as described below.

Correlation of Immunogenicity with Degenerate Binding Capacity

Pursuant to the identification of peptides with degenerate binding capacity, whether levels of DR degeneracy could be associated with immunogenicity was explored. The basis for this analysis comes from the observations in the context of HLA class I, where it was noted that the most immunogenic peptides are often the most degenerate, and vice versa (see, e.g., Doolan, D. L., et al. (2000) J. Immunol., 165:1123-37). For this analysis, a panel of epitopes of known HLA-DR restriction was assembled, and tested for binding against a panel of 15 MHC class II molecules (see, Table 6). Most of analyzed epitopes were described in the literature with reported MHC restriction element(s) (See, e.g., Anderson, D. C., et al. (1988) Science, 242:259-61; Bocchia, M., et al, (1996) Blood, 87:3587-92; Celis, E., et al. (1988) J. Immunol., 141:2721-28; Dayan, C., et al. (1991) Proc. Natl. Acad. Sci. USA., 88:7415-19; De Magistris, M. T., et al. (1989) J. Exp. Med., 169:1519-32; Ferrari, C., et al. (1989) J. Clin. Invest., 84:1314-1319; Lamonaca, V., et al. (1999) Hepatology, 30:1088-98; Markovic-Plese, S., et al. (1995) J. Immunol., 155:82-92; Oftung, F., et al. (1988) J. Immunol., 141:2749-54; Oftung, F., et al. (1990) J. Immunol., 144:1478-83; Panina-Bordignon, P., et al. (1989) Eur. J. Immunol., 19:2237-42; Perez, M., et al. (1990) J. Biol. Ches., 265:16210-15; Rothbard, J. B. and Taylor, W. R. (1988) EMBO J., 7:93-100; Rothbard, J. B., et al. (1988) Cell, 52:515-23; Sinigaglia, F., et al. (1988) Nature, 336:778-80; Topalian, S. L., et al. (1996) J. Exp. Med., 183:1965-71; Valli, A., et al. (1993) J. Clin. Invest., 91:616-28; and Van Schooten, W. C., et al. (1989) Eur. J. Immunol., 19:2075-79). Each of the peptides bound the MHC molecule reported as the restriction element with an affinity of 1000 nM or less. Of the 48 reported restriction events, 21 (65%) were associated with degenerate binding, as defined by a hit rate of 33% or 5/15 molecules tested. We therefore set a threshold of 5 molecules as the minimum required to be considered degenerate.

Identification of Degenerate Regions in Candidate Molecules

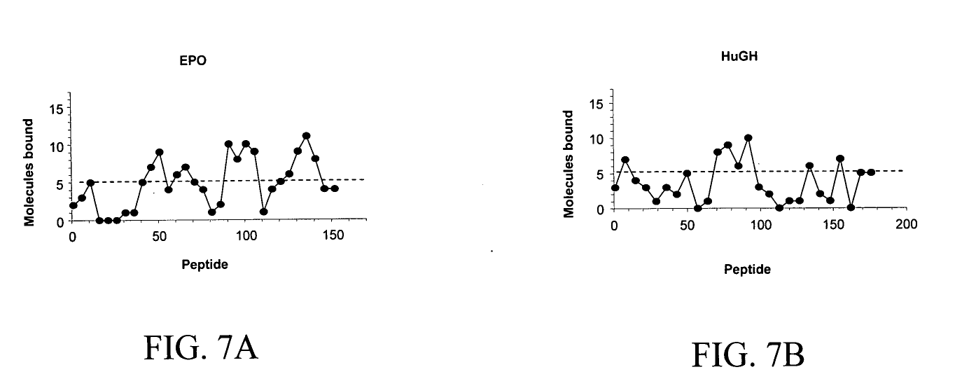

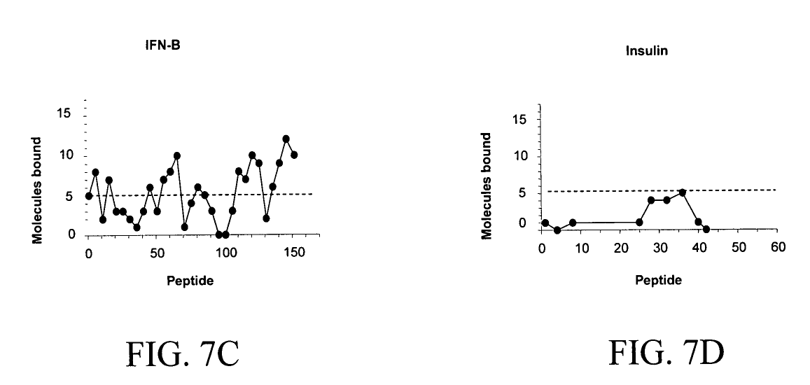

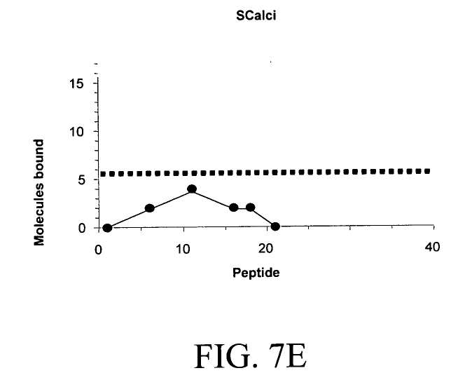

Following the above analysis we went on to determine the degenerate regions that were present in all five of our candidate proteins. In FIG. 7, we show the location of degenerate peptides for each protein tested. While insulin and calcitonin did not contain any degenerate binding regions, several regions were identified within EPO, IFNβ and hGH. A summary of all the degenerate regions identified is shown in Table 7.

Immunogenicity of Candidate Proteins

As described previously, we selected five candidate proteins for validation of our ImmunoStealth™ technology based on evidence of immunogenicity from literature. Our approach was to determine the immunogenic potential of these candidate proteins using our in vitro systems. Our binding analysis indicated that EPO, hGH and IFNβ were the molecules that contained the most degenerate binding regions and therefore were suitable candidates for validation. Upon further analysis IFNβ was excluded from the study at this point, due to the anti-proliferative nature of the molecule itself. We therefore evaluated the immunogenicity of only EPO and hGH using in vitro model systems. For these in vitro studies, we obtained peripheral blood mononuclear cells from a panel of normal donors. MHC class II typing of the donors used in this study was carried out using PCR based sequencing techniques (Table 8A). As seen, the donor pool we had chosen had a representation of several different MHC haplotypes and was not biased towards one or the other MHC molecules. The PBMC from these normal donors were stimulated in vitro with autologous antigen-pulsed dendritic cells followed by multiple stimulations with antigen pulsed autologous PBMCs. Immune responses were measured following multiple stimulations using and IFNγ based ELISPOT assay. The number of donors tested for immunogenicity ranged from 5 in case of growth hormone to 7 in the case of erythropoietin. As indicated in (Table 8B), immune responses were observed immune responses were obtained in 0/5 donors (0%) for hGH and 5/(71%) donors in the case of erythropoietin.

Antigenicity Analysis of Overlapping Erythropoietin Peptides

As indicated in the previous section, T cell lines with immune reactivity against erythropoietin were obtained in 5 different donor lines. These lines were thawed from the freezer and stimulated one additional time in vitro before antigenicity analysis was carried out. Before carrying out the analysis, the lines were tested for retention of immune reactivity against EPO. Antigenicity analysis for each of the five donor lines was carried out independently. All the 31 native erythropoietin peptides and the appropriate controls were tested in these assays.

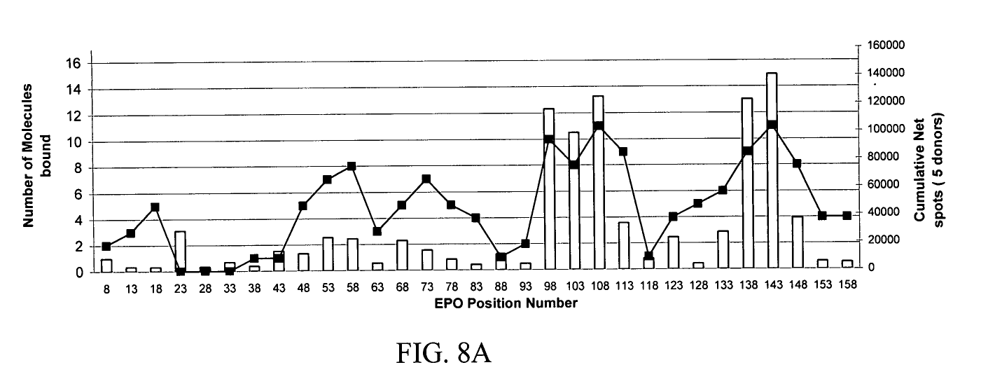

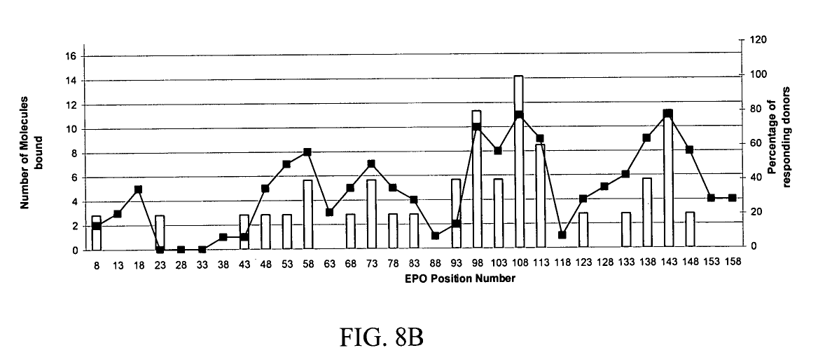

FIG. 8A depicts the magnitude of response obtained against each individual peptide. The cumulative net spots against each individual are shown as bar graphs on the primary y axis. The line graph is a representation of the binding analysis performed in Table 5A, where the number of MHC molecules bound by each peptide as indicated on the secondary y axis. These data clearly demonstrate that there were two highly immunogenic regions within the erythropoietin protein corresponding to EPO position numbers (98-108) and the regions (138-148). While some other positive responses (in 2/5 donors) were obtained against peptides 58, 73, 93, 113, the magnitude of response was significantly lower than those obtained to regions 98-108 and 138-148.

The frequency of responses obtained against all overlapping erythropoietin peptides are indicated in FIG. 8B. The bar graphs on the primary y axis represent the percentage of donors in which a positive ELISPOT response was obtained based on our rather stringent criteria for a positive ELISPOT response described in materials and methods. This analysis demonstrates the highest frequency of response (80-100%) was against peptides corresponding to EPO position numbers 98, 108 and 143, while lower frequencies of response was obtained against several other peptides. Once again this finding corroborates that EPO peptides (98-108) (SEQ ID NO:40) and (138-148) (SEQ ID NO:47) are not only immunodominant but also highly degenerate and therefore, are likely to be immunogenic in a large percentage of the worldwide population.

All the antigenic peptides (defined as peptides generating a positive response in ≧2 donors) are shown in Table 9. As seen, 9 antigenic peptides were identified in the erythropoietin molecule; of these 9, 8 peptides were identified by our MHC binding analysis, and 6 were identified by our in silico analysis using a sensitivity of 125 nM. Thus, our MHC binding analysis is highly sensitive in that it is capable of identifying 90% of the immunogenic peptides. In the case of the algorithms the sensitivities vary between 66-78%. This analysis emphasizes the need for performing actual MHC binding analysis as it is not only correlative of immunogenicity but also provides an empirical basis to determine the success of the analogs generated for the purpose of removing immunogenic epitopes from protein or antibody molecules.

Immunodominant EPO Peptides are Presented by HLA DR Molecules

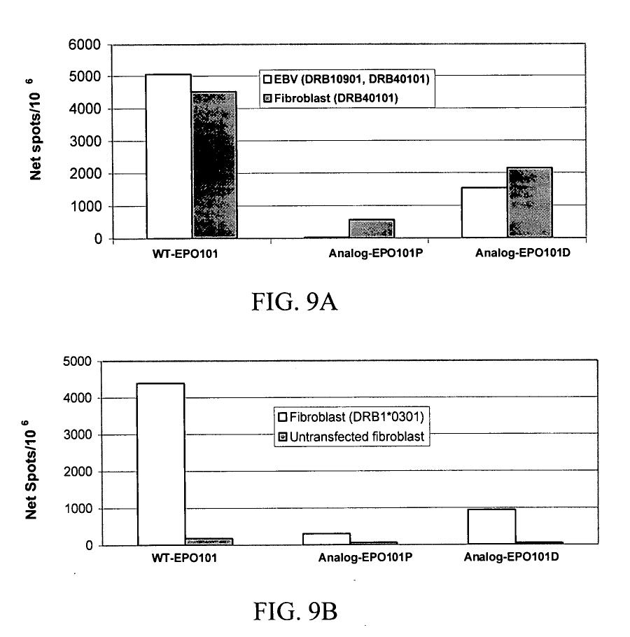

To confirm that the immunodominant epitopes of erythropoietin are indeed presented by HLA DR molecules we carried out a limited study. In this study, we took T cell lines obtained from two donors (donor 1 and donor 2) and measured immune responses to the immunodominant peptide EPO 98-108 (SEQ ID NO:40) against peptide pulsed fibroblasts. The fibroblasts used for this study were single transfectants of the HLA-DR molecule expressed by the corresponding donor cells. In case of Donor 1 (DRB1*0901, DRB4*0101), the single transfected fibroblast of DRB1*0901 was not available therefore we used an EBV transformed cell line instead. In case of Donor 2 (DRB1*0301) the single transfectant expressing DRB1*0301 was used. As seen in FIG. 9, the erythropoietin peptide EPO 108 is presented by both fibroblast transfectants and EBV cells carrying the relevant DR molecules. Untransfected fibroblasts or EBV cells were not able to present the EPO peptide. These results indicate that analogs aimed at reducing HLA-DR binding capacity should have significant impact on reducing immunogenicity. In fact, it is shown in FIG. 9 that while the wildtype peptide EPO 108 is presented well by the fibroblast the analogs EPO 108P and analog EPO 108D (identified as potential analogs in Table 8A were not presented by the DR molecules as binding to these molecules was disrupted in the analogs.

Analoging to Reduce Degeneracy

To determine if it is possible to analog peptides to effectively reduce their degeneracy, we selected the most degenerate regions of EPO for further analysis. Because analysis of all of the possible analogs that could be made for each 15-mer peptide is impractical, an analoging strategy based on PIC analysis was developed.

This strategy entailed identifying the main 9-mer core region of each degenerate 15-mer using PIC algorithm analysis. In general, for DR binding the residue in position 1 of the main core is the strongest determinant of binding capacity (Rammensee, Bachmann & Stevanovic (1997) MHC ligands and peptide motifs (Landes Bioscience, Heidelberg); Rammensee, H. G., et al. (1995) Immunogenetics, 41:178-228; Rammensee, H., et al. (1999) Immunogenetics, 50:213-19); Southwood, S., et al. (1998) J. Immunol., 160:3363-73; Wiley, et al., EMBO J., (1990) 9(6):1797-1803-DR1). For this reason, a comprehensive set of analogs were made of the P1 residue. Because P1 anchors are most frequently F, W, Y, L, I, V or M, analogs containing these residues were not made. Also, cysteine was avoided due to possible disulfide bond formation, which makes C containing peptides difficult to work with. The remaining 12 amino acid residues were used for substitution analysis. At the remaining positions of the main 9-mer core region, D, R, and G analogs (identified by PIC as those substitutions which most frequently be associated with a deleterious affect) were made. Finally, PIC was utilized to identify an additional 9-mer core that may be function as a DR binding region. The P1 position of this secondary core was analoged with the same panel of substitutions used for the P1 position of the main core region.

To determine which analogs would render the protein less or non-immunogenic, we tested each of the modified peptides for binding against the panel of 17 MHC class II molecules. Results for the EPO epitopes are shown in Table 10A-B. Selected analog candidates were chosen based on the amount of molecules which no longer bound the analog (comp ratio ≦1.000) and the number of molecules in which there was at least a 10 fold reduction in binding. Ten analogs from EPO 101 and eight analogs from EPO 136 were selected based on a comparative ratio of less than 1.000 and a 10 fold reduction in binding to 5 or more molecules, listed in Tables 11A-11B. These candidates were then further pursued in immune assays.

Antigenicity of EPO Analogs with Reduced Binding

As described previously, binding analysis was carried out on a large number of EPO analogs. For antigenicity analysis only a subset of these analogs (identified in Tables 11A-11B that had 10× reduction in binding in 5 of more HLA molecules were chosen for the analysis. ELISPOT responses in four donor lines were measured against analog pulsed PBMC (Table 12). Wildtype peptide responses were measured in the same assay as controls. As seen in all four donors wildtype responses were obtained against the four donors, whereas the responses against the analogs varied from 0 of 4 to 4 of 4. The scale of analog responses was variable varying from + to +++. A response was considered disrupted if the scale of response was − or a +. The most promising analogs for the purpose of reduced immunogenicity are indicated by shaded regions and correspond to the sequences GLRSLTDLLRALGAQ (SEQ ID NO:187), GPRSLTTLLRALGAQ (SEQ ID NO:159), DTFRKLFRVYDNFLR (SEQ ID NO:_{—}233) and DTFRKDFRVYSNFLR (SEQ ID NO:199).

Structural Analysis of Analogs

The predicted bioactivity of erythropoietin variants was based on analysis of structure/function data, evolutionary conservation of variation and analysis of the predicted structures of erythropoietin variants. Functionally important domains and residues have been identified by comparing native human erythropoietin with erythropoietin variants in vitro in cell proliferation assays (Wen, et al. (1994) J. Biol. Chem., 269:22839, Matthews, et al. (1996) Proc. Natl. Acad. Sci. U.S.A., 93:9471-9476), by cell or erythropoietin receptor binding competition assays (Matthews, et al. (1996) Proc. Natl. Acad. Sci. U.S.A., 93:9471-9476), or by comparative immunoreactivity with monoclonal antibodies (Elliot S., et al. (1996) Blood, 87:2702). These studies show that 70% of substitutions of amino acids located within the erythropoietin receptor contact sites (Syed R. S., et al. (1998) Nature, 395:511) result in a decrease in bioactivity compared with just 6% for substitutions of amino acids outside of the contact sites (Table 13A). Therefore, substitutions outside of the receptor contact sites would be predicted to be more likely to retain their in vivo bioactivity than those located within the critical receptor contact sites.

In addition to evaluation of the functional information available for erythropoietin variants, the primary amino acid sequences of mammalian erythropoietin proteins and protein structures of erythropoietin structural neighbors were aligned (Altschul, S. F., (1997), Nucl. Acids Res., 25:3389-3402, Holm, L., and Sander, C., (1996) Science, 273(5275):595-60) in order to evaluate conservation of particular residues in the erythropoietin sequence or structure as one metric for the importance of that particular residue to erythropoietin function.

Proposed experimental erythropoietin amino acid substitutions in the erythropoietin peptides G101-Q115 (GLRSLTTLLRALGAQ) (SEQ ID NO:40) and D136-R150 (DTFRKLFRVYSNFLR) (SEQ ID NO:47), were at positions Leu-102, Arg-103, Ser-104, Leu-105, Thr-107, Leu-141, Arg-143, Val-144, Ser-146 and Asn-147. Variation was seen in the primary mammalian sequences for Leu-105, Thr-107, Val-144 and Ser-146 although substitutions at these positions were generally quite conservative (L105I (macaque), T107S (cat, cow, mouse, rat, sheep, pig, rabbit, dog), S146A (mouse, sheep, pig), V144N (mouse homolog, pig), V144I (cat, cow, sheep, mouse homolog, rabbit, dog)). These data suggests that substitutions at amino acid positions Leu-105, Thr-107 and Ser-146 naturally allow for conservative variation while still retaining bioactivity, and Val-144 may allow retention of bioactivity with even greater flexibility in substitutions.

Comparison of structural neighbors for the 5 residues in peptide G101-Q115 showed that Leu-102 and Leu-105 were very well conserved with most residues in the structural neighbors preserving the aliphatic non-polar nature of these positions. Less conservation was seen for Arg-103 (although 9 of 11 residues were polar, only 2 were also basic residues). A similar level of conservation was seen for Thr-107 with 7 of 11 polar residues. In contrast, little conservation was seen for Ser-104 with 4 of 11 polar residues at this position. For experimental variants within the peptide D136-R150 (DTFRKLFRVYSNFLR) (SEQ ID NO:47), all residues except Val-144 were well conserved in the structural neighbors. Residues at the same position as erythropoietin Leu-141 were mostly nonpolar (9 of 11) residues with 5 aliphatic nonpolar residues in this position. Arg-143 had 9 of 11 polar and 4 basic residues in this position. Ser-146 and Asn-147 had polar residues in this position in all structures analyzed. These data would suggest that some residues may be more important for maintaining the structure (L102, L105, L141, R143, S146, and N147) than others (S104 and V144) and that conservative substitution of these residues would not be expected to disrupt the protein structure.

The published and proposed experimental erythropoietin variants were modeled using the erythropoietin structure 1EER:A as the template structure for model building. The predicted erythropoietin structures were then evaluated for potential structural consequences of the proposed experimental substitutions. With the exception of the substitution of proline at Leu-102 (root square mean deviation of 0.55 Å), the proposed experimental substitutions had little effect on the gross predicted structures of the erythropoietin variant proteins with root mean square deviations for the main backbone of 0.14 Å or less. Taken together, these analyses suggest that some of the proposed experimental substitutions within erythropoietin peptides G101-Q115 and D136-R150 will result in erythropoietin variants that are expected to retain wild-type bioactivity in vivo (Table 13B). For example, substitution of Leu-102 with alanine or glycine should not disrupt bioactivity. Leu-102 is predicted to be within the buried surface of helix C, therefore substitutions with either glutaric acid or serine at this position may have a structural consequence which could affect bioactivity (Elliot S., et al. (1996) Blood 87:2702). However, with the exception of the proline substitution, none of the Leu-102 substitutions are predicted to disrupt the structure significantly and Leu-102 has not been identified as critical for erythropoietin bioactivity.

In contrast to Leu-102, Arg-103 and Ser-104 are residues that have been identified as critical for receptor activation. These residues are part of the low-affinity receptor binding site. All amino acid substitutions at Arg-103 and Ser-104, with the exception of lysine substitution of Arg-103, significantly reduced erythropoietin bioactivity. Interestingly, erythropoietin with alanine substituted for Arg-103 retains the ability to bind to the receptor, but fails to activate it.

Leu-105 is a structurally conserved residue in erythropoietin structural neighbors and is predicted to be within the buried surface of helix C. Changing this residue to serine, a polar residue, reduces bioactivity of the erythropoietin variant protein. Also, substitutions of Leu-105 with aspartic acid have increased immunoreactivity to 9G8A (8600%) suggesting that the conformation of the variant erythropoietin has been affected (more denatured). Therefore, it is likely that substituting aspartic acid, an acidic polar residue, at this position will result in a reduction in erythropoietin bioactivity.

Thr-107 is predicted to be on the surface of helix C and is not located in a receptor contact site. Biological activity was maintained with both polar and nonpolar substitutions at this position, therefore substituting aspartic acid at this position is unlikely to disrupt erythropoietin bioactivity in vivo.

Leu-141 is predicted to be buried in helix D and most erythropoietin structural neighbors have a nonpolar residue in this position. This would suggest that substituting Leu-141 with aspartic acid may have a structural consequence. However, this residue is not in a critical receptor binding site and no gross deviations in the modeled structure were detected, therefore it is predicted that the aspartic acid substitution for Leu-141 will have wild-type to near wild-type bioactivity.

Arg-143 is predicted to be located on the surface of helix D within the high affinity receptor binding site. Aspartic acid substitution of Arg-143 is expected to retain wild-type bioactivity as a similar erythropoietin variant with substitution of glutamic acid for this residue retains wild-type bioactivity in vitro. The modeled protein structure of the erythropoietin protein with glycine in the place of Arg-143 maintained the structure and hydrogen-bonding pattern of the wild-type protein; therefore this substitution should have near wild-type to possibly wild-type bioactivity.

Erythropoietin variants with aspartic acid, glycine or arginine substituted for Val-144 are expected to that have wild-type bioactivity. This prediction is based on the predicted wild-type structure seen with these variants, the predicted location on the surface of helix D outside of the receptor interaction site, the variation of this residue in nature (polar and nonpolar residues in mammalian erythropoietin proteins and lower conservation in structural neighbors), and that erythropoietin variant proteins containing both polar and nonpolar substitutions are bioactive.

Substituting Ser-146 with aspartic acid is predicted to result in a variant erythropoietin protein that will have near wild-type to wild-type bioactivity. This prediction is based on a predicted wild-type structure, the presence of this residue on the surface of helix D outside of the receptor interaction site, and that erythropoietin variant proteins with both polar and nonpolar amino acids at this position maintain wild-type bioactivity.

Asn-147 is located in the high-affinity receptor binding site and forms three hydrogen bonds with the EPO receptor residues, and substitution of this residue with either lysine or alanine results in a reduction of erythropoietin bioactivity in vitro. Therefore, substituting Asn-147 with aspartic acid will most likely result in lower bioactivity of the resulting erythropoietin variant protein.

Improving Analoging Strategies

Additional studies were performed to determine if binding capacities against HLA molecules could be further reduced by combining different analogs. For this study, we generated 3 analogs that carried two amino acid substitutions each. The binding analysis for these three double analogs (Tables 14A-B) was compared to that of single analogs. The double analogs were significantly more efficient at reducing binding capacity to multiple HLA molecules. The double analog of the region EPO 101 degenerate region GGRSLTDLLRALGAQ (SEQ ID NO:254) and GSRSLTDLLRALGAQ (SEQ ID NO:255) bound to 2 of 17 or 3 of 17 molecules compared to the single analogs GGRSLTTLLRALGAQ (SEQ ID NO:155), GSRSLTTLLRALGAQ (SEQ ID NO:162) or GLRSLTDLLRALGAQ (SEQ ID NO:187) that bound to 8 of 17, 8 of 17, or 5 of 17 molecules respectively. Moreover, the binding capacities were reduced by tenfold or more in either 13 or 14 molecules in the case of double analogs compared to 7 or 8 molecules with the single analogs.

Similarly, the double analog DTFRKDFRVYDNFLR (SEQ ID NO:257) of the EPO 136 degenerate region bound to only 3 of 17 molecules whereas the single analogs DTFRKDFRVYSNFLR (SEQ ID NO:199) and DTFRKLFRVYDNFLR (SEQ ID NO:233) bound to 9 of 17 or 7 of 17 molecules respectively. Also the binding capacity was reduced by ten-fold or more in 11 of 17 molecules for the double analog versus 8 of 17 or 5 of 17 molecules for the corresponding two single analogs.

Antigenicity analysis was carried out on these three double analogs, two of the EPO 101 region peptides and one of the EPO 136 degenerate region peptides in four different donors as shown in Table 14C. All three of these double analogs were very successful in disrupting immune responses in the donors tested. The frequency of disruption ranged from 3 of 4 to 4 of 4 donors examined. The frequency of disruption was not significantly different from the corresponding single analogs. However, the very fact that these double analogs will likely bind to a significantly lower number of MHC molecules when more than four donors are examined, means that they are likely to be less immunogenic than the single analogs in view of the large number of MHC molecules that will be encountered when a protein or antibody drug is administered to the various populations of the world.

Substitutions for Modified EPO Proteins

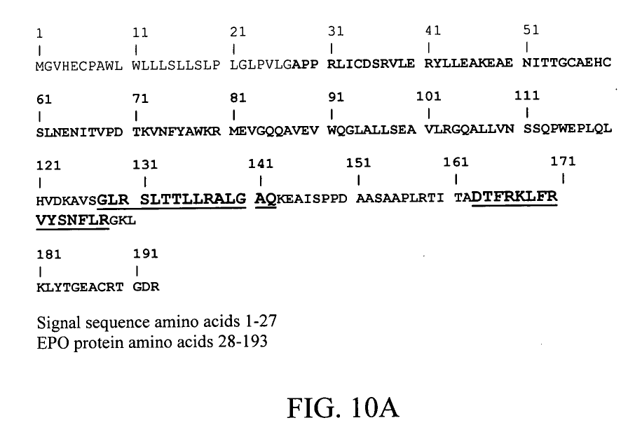

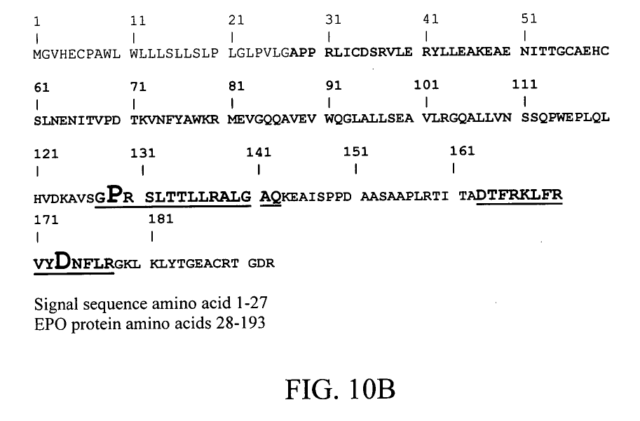

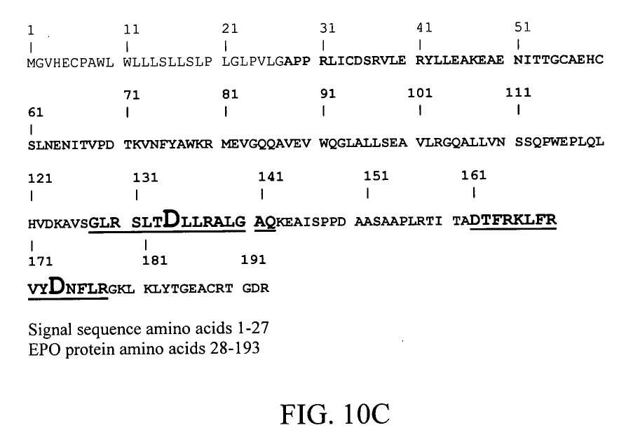

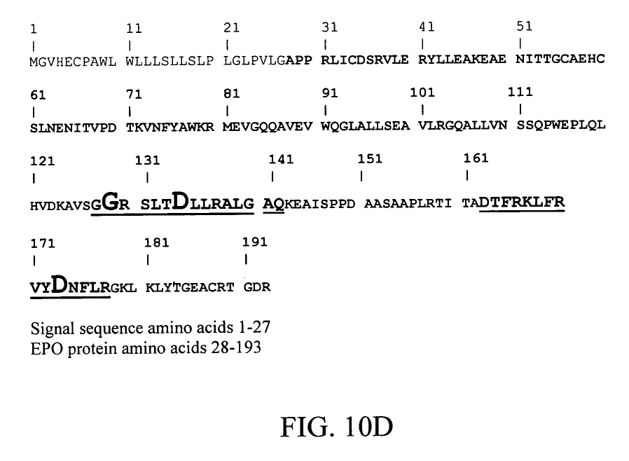

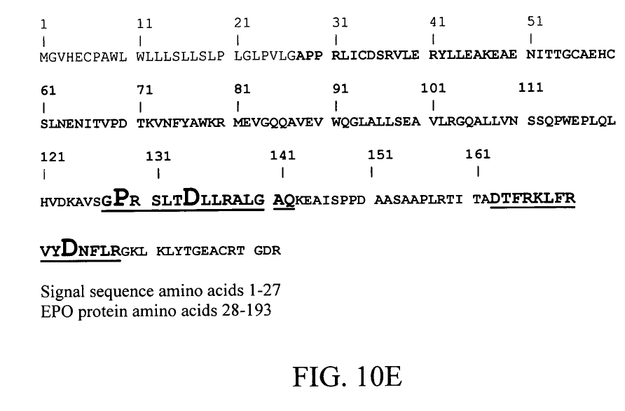

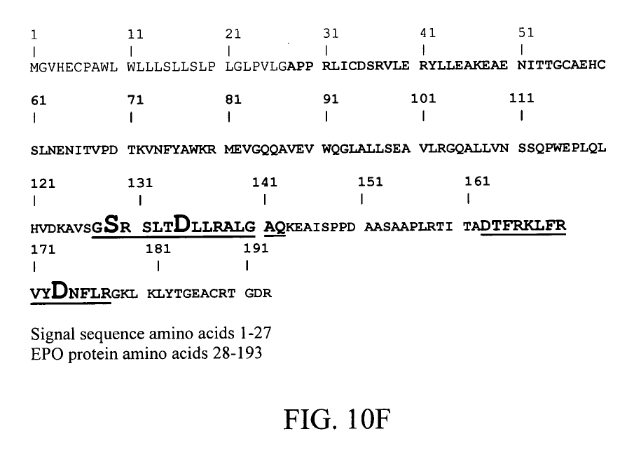

We have compiled a list of substitutions to be incorporated into the native erythropoietin molecules with the specific purpose of reducing its immunogenicity. The factors that were taken into account included antigenicity of the analogs, their impact on the structure of the molecules determined by structural modeling, the number of MHC molecules bound, the number of molecules in which there was 10× reduction in binding, targeting of both the immunodominant EPO regions EPO 101 (SEQ ID NO:40) and EPO 136 (SEQ ID NO:47) as well as creation of any additional binding sites. The five modified erythropoietin molecules to be generated are shown in FIGS. 10A-F. The two immunodominant regions are shown in bold and are underlined, while the substitutions generated are indicated in a larger font.

These experiments confirm that issues with unwanted immune responses exist with the use of therapeutic proteins. These include both non-antibody and antibody molecules. Presently, few approaches exist for successfully reducing immunogenicity of proteins. For example, PEGylation was not successful in a recent clinical trial on PEG-rHuMGDF. This trial was discontinued due to development of neutralizing cross-reactive antibodies against the parent molecule). Herein, we describe the development of “ImmunoStealth™”, an integrated bioinformatics, biochemical and cellular immunology approach that specifically addresses the issue of unwanted immune responses against protein therapeutics.

For validation of ImmunoStealth, 5 different candidate molecules (Calcitonin, Insulin, Human growth hormone, Interferon B and Erythropoietin) were chosen based on an evidence of immunogenicity against these molecules. Large sets of overlapping peptides spanning the entire sequence of the native molecules were synthesized and tested in our MHC binding assays to determine which regions of the various molecules were the highest and most degenerate binders and therefore would most likely be the “offending” epitopes. Our binding analysis was compared to the efficiency of the various in silico systems that are available for prediction of epitopes.

In order to access the efficiency of our binding analysis to actual immune responses against the candidate proteins we carried out immunogenicity testing of two of these candidate proteins (human growth hormone and erythropoietin that had the most degenerate peptide binding regions) using in vitro systems. Two highly immunogenic regions identified in the erythropoietin molecules and both these regions were predicted by our binding analysis to be the most immunogenic regions. These results demonstrate that our binding analysis accurately predicts immunogenic regions in a native protein.

The two degenerate regions predicted by binding and immunogenicity studies were modified to determine whether specific changes made in these regions would reduce their immunogenicity. Several different single and double substitution analogs were generated, and binding analysis was carried out to identify the analogs with a reduced capacity to bind to multiple Class II molecules. In parallel, these analogs were also tested for their capacity to evoke an immune response against native erythropoietin-specific T cell lines. These experiments resulted in the identification of several different analogs that were very successful in disrupting immune responses in multiple donors and had substantially reduced binding capacities against multiple HLA Class II molecules and therefore would be associated with reduced immunogenicity when the changes are incorporated into the native protein sequence.

Finally, we carried out a structural analysis of some of the “identified” analogs to evaluate what effect if any these substitutions would have on the structure of the native protein. Moreover, a careful analysis was done to determine if any if of the proposed substitutions were in receptor contact regions and that may have significant impact on biological activity of the native molecule. This analysis resulted in the selection of potential analogs with greatly reduced binding capacity to multiple HLA molecule, reduced antigenicity, as well as minimal predicted impact on the structure of the native erythropoietin molecule, and therefore were most likely to be biologically active.

### Example 2

### Generation of Modified EPO Construct Inserts to be Used in the Construction of Bacterial and Eukaryotic Expression Vectors

Following the identification and modification of immunodominant HTL epitopes in a peptide, polypeptide, protein, and/or antibody of the invention, one of skill in the art can subclone a full-length DNA sequence that encodes the modified peptide, polypeptide, protein, and/or antibody sequence into a bacterial expression vector. Many appropriate vectors are available commercially and are well-known to those of skill in the art.

The following protocol may be used to express Modified EPO Constructs 1-5 of the present invention in a bacterial protein expression system. PCR is used to generate a full-length copy of each of the Modified EPO Construct coding sequences. The Modified EPO Construct inserts are then cloned into either a bacterial and/or eukaryotic expression vectors.

Briefly, the process is begun with a wild type EPO construct in pCRblunt (Invitrogen, Carlsbad, Calif.). To produce the Modified EPO Constructs, the following mutations will be introduced.

| | aa 129 | aa 134 | aa 173 |
|---|---|---|---|
| EPO wt | L | T | S |
| EPO 1 | P | T | D |
| EPO 2 | L | D | D |
| EPO 3 | G | D | D |
| EPO 4 | P | D | D |
| EPO 5 | S | D | D |

Essentially, the mutants were made by using overlapping complementary oligonucleotides encoding the analog mutations in a PCR assay with the wildtype EPO or EPO1 (once generated) as template and the proof-reading polymerase Pfu (Stratagene). Blocks encoding the new sequences were generated with 20 nucleotide overlap and then annealed together and extended in a gene synthesis reaction to synthesize full-length EPO. The analog EPOs were then cloned into pCRblunt and sequenced at Retrogen, Inc. (San Diego, Calif.). The 5′ (EP1) and 3′ (EP5) oligos encode the restriction sites Hin dIII and Bam H1, respectively, to facilitate future subcloning.

Because the mutation in position 173 is in all the mutants, it is generated first, and then used as a template to introduce all the other mutations. Use EPO wt as a template with oligos for EPO 1 to introduce mutations by PCR. Once correct EPO 1 identified in pCRblunt, use that as a template for subsequent reactions. Long oligos are resuspended at 25 pmol/μl, and short oligos are resuspended at 250 pmol/μl for stocks, then make 25 pmol/μl for ‘in use’ stocks. The individual PCR reactions are set up as follows:

| 1. | For EPO 1: | |
|---|---|---|
| | a. | Set up PCR reaction for blocks: |
| | BLOCK A: | |
| | | 1 μl EPO wt DNA |
| | | 1 μl EP-1 oligo (25 pmol/μl) (SEQ ID NO: 263) |
| | | 1 μl EP-3 oligo (25 pmol/μl) (SEQ ID NO: 265) |
| | | 10 μl 10× pfu buffer |
| | | 2.5 mM dNTP |
| | | 1 μl PFU enzyme |
| | | 83.5 μl H₂O |
| | | Σ 100 μl |
| | BLOCK B: | |
| | | 1 μl EPO wt DNA |
| | | 1 μl EP-2 oligo (25 pmol/μl) (SEQ ID NO: 264) |
| | | 1 μl EP-4 oligo (25 pmol/μl) (SEQ ID NO: 266) |
| | | 10 μl 10× pfu buffer |
| | | 2.5 mM dNTP |
| | | 1 μl PFU enzyme |
| | | 83.5 μl H₂O |
| | | Σ 100 μl |
| | | Check T_{M} for oligos and run 30-cycle PCR at appropriate |
| | | temperature. |
| | | 94° C., 30 sec. |
| | | 58° C., 30 sec. |
| | | 72° C., 1 min. |
| | | 30 cycles |
| | b. | Run PCR products out on a gel. Gel purify, elute in 8 μl (or |
| | | accordingly based on intensities). |
| | c. | Set up Gene-Synthesis reaction to put together Block A and Block B: |

| 4 μl Block A | |
|---|---|
| 4 μl Block B | |
| 5 μl 10× pfu buf | |
| 2 μl dNTP | |
| 1 μl PFU | |
| 34 μl H₂O | |
| Σ 50 μl | |
| Run GeneSyn (5 + 10) cycle, use annealing temperature | |
| based on overlap region (ie. EP-2, EP-3). | |
| 5 cycles: | 94° C., 30 sec. |
| | 58° C., 30 sec. |
| | 72° C., 1 min. |
| 10 cycles: | 94° C., 30 sec. |
| | 72° C., 1 min. |

| | d. | Amplify Block AB (entire EPO 1) with oligos EP-1 (SEQ ID NO: 263) |
|---|---|---|
| | | and EP-5 (SEQ ID NO: 267): |
| | | 10 μl Block AB |
| | | 1 μl EP-1 (25 pmol/μl) (SEQ ID NO: 263) |
| | | 1 μl EP-5 (25 pmol/μl) (SEQ ID NO: 267) |
| | | 10 μl 10× pfu buffer |
| | | 2.5 mM dNTP |
| | | 1 μl PFU enzyme |
| | | 74.5 μl H₂O |
| | | Σ 100 μl |
| | | Run 30-cycle PCR, use annealing temperature based on oligos EP- |
| | | 1 (SEQ ID NO: 263) and EP-5 (SEQ ID NO: 267). |
| | e. | Run PCR product out on agarose gel, gel purify, elute in 8 μl. |
| | f. | Clone EPO 1 PCR product into pCRblunt: |
| | | 1 μl pCRblunt vector (Invitrogen, 25 ng/μl) |
| | | 4 μl EPO 1 insert |
| | | 1 μl 10× T4 DNA ligase buffer |
| | | 1 μl T4 DNA ligase |
| | | 3 μl H₂O |
| | | Σ 10 μl |
| | | Incubate 15° C., 2 h to O/N. |
| | | Transform TOP10 cells with 2 μl of ligation. |
| | | Plate on KANAMYCIN plates. |
| | g. | Screen colonies with T7 (SEQ ID NO: 276) and M13r (SEQ ID |
| | | NO: 277) oligos. |
| | h. | Make spin prep of 3 positives |
| | i. | Sequence 3 positives. |
| | j. | Make glycerol stock of favorite and use DNA as template for the other |
| | | mutants. |
| 2. | For EPO 2: | |
| | a. | Set up PCR reaction for blocks: |
| | BLOCK A: | |
| | | 1 μl EPO 1 DNA |
| | | 1 μl EP-1 oligo (25 pmol/μl) (SEQ ID NO: 263) |
| | | 1 μl EP-7 oligo (25 pmol/μl) (SEQ ID NO: 269) |
| | | 10 μl 10× pfu buffer |
| | | 2.5 mM dNTP |
| | | 1 μl PFU enzyme |
| | | 83.5 μl H₂O |
| | | Σ 100 μl |
| | BLOCK B: | |
| | | 1 μl EPO 1 DNA |
| | | 1 μl EP-6 oligo (25 pmol/μl) (SEQ ID NO: 268) |
| | | 1 μl EP-5 oligo (25 pmol/μl) (SEQ ID NO: 267) |
| | | 10 μl 10× pfu buffer |
| | | 2.5 mM dNTP |
| | | 1 μl PFU enzyme |
| | | 83.5 μl H₂O |
| | | Σ 100 μl |
| | | Check T_{M} for oligos and run 30-cycle PCR at appropriate |
| | | temperature. |
| | k. | Run PCR products out on a gel. Gel purify, elute in 8 μl (or |
| | | accordingly based on intensities). |
| | l. | Set up Gene-Synthesis reaction to put together Block A and Block B: |
| | | 4 μl Block A |
| | | 4 μl Block B |
| | | 5 μ 10× pfu buf |
| | | 2 μl dNTP |
| | | 1 μl PFU |
| | | 34 μl H₂O |
| | | Σ50 μl |
| | | Run GeneSyn (5 + 10) cycle, use annealing temperature |
| | | based on overlap region (i.e., EP-6, EP-7). |
| | m. | Amplify Block AB (entire EPO 2) with oligos EP-1 (SEQ ID NO: 263) |
| | | and EP-5 (SEQ ID NO: 267): |
| | | 10 μl Block AB |
| | | 1 μl EP-1 (25 pmol/μl) |
| | | 1 μl EP-5 (25 pmol/μl) |
| | | 10 μl 10× pfu buffer |
| | | 2.5 mM dNTP |
| | | 1 μl PFU enzyme |
| | | 74.5 μl H₂O |
| | | Σ 100 μl |
| | | Run 30-cycle PCR, use annealing temperature based on oligos EP- |
| | | 1 (SEQ ID NO: 263) and EP-5 (SEQ ID NO: 267). |
| | n. | Run PCR product out on agarose gel, gel purify, elute in 8 μl. |
| | o. | Clone EPO 2 PCR product into pCRblunt: |
| | | 1 μl pCRblunt vector (Invitrogen, 25 ng/μl) |
| | | 4 μl EPO 2 insert |
| | | 1 μl 10× T4 DNA ligase buffer |
| | | 1 μl T4 DNA ligase |
| | | 3 μl H₂O |
| | | Σ 10 μl |
| | | Incubate 15° C., 2 h to O/N. |
| | | Transform TOP10 cells with 2 μl of ligation. |
| | | Plate on KANAMYCIN plates. |
| | p. | Screen colonies with T7 (SEQ ID NO: 276) and M13r (SEQ ID |
| | | NO: 277) oligos. |
| | q. | Make spin prep of 3 positives |
| | r. | Sequence 3 positives. |
| | s. | Make glycerol stock of favorite. |
| 3. | For EPO 3: | |
| | a. | Set up PCR reaction for blocks: |
| | BLOCK A: | |
| | | 1 μl EPO 1 DNA |
| | | 1 μl EP-1 oligo (25 pmol/μl) (SEQ ID NO: 263) |
| | | 1 μl EP-9 oligo (25 pmol/μl) (SEQ ID NO: 271) |
| | | 10 μl 10× pfu buffer |
| | | 2.5 mM dNTP |
| | | 1 μl PFU enzyme |
| | | 83.5 μl H₂O |
| | | Σ 100 μl |
| | BLOCK B: | |
| | | 1 μl EPO 1 DNA |
| | | 1 μl EP-8 oligo (25 pmol/μl) (SEQ ID NO: 270) |
| | | 1 μl EP-5 oligo (25 pmol/μl) (SEQ ID NO: 267) |
| | | 10 μl 10× pfu buffer |
| | | 2.5 mM dNTP |
| | | 1 μl PFU enzyme |
| | | 83.5 μl H₂O |
| | | Σ 100 μl |
| | | Check T_{M} for oligos and run 30-cycle PCR at appropriate |
| | | temperature. |
| | t. | Run PCR products out on a gel. Gel purify, elute in 8 μl (or |
| | | accordingly based on intensities). |
| | u. | Set up Gene-Synthesis reaction to put together Block A and Block B: |
| | | 4 μl Block A |
| | | 4 μl Block B |
| | | 5 μl 10× pfu buf |
| | | 2 μl dNTP |
| | | 1 μl PFU |
| | | 34 μl H₂O |
| | | Σ 50 μl |
| | | Run GeneSyn (5 + 10) cycle, use annealing temperature |
| | | based on overlap region (i.e., EP-8, EP-9). |
| | v. | Amplify Block AB (entire EPO 3) with oligos EP-1 (SEQ ID NO: 263) |
| | | and EP-5 (SEQ ID NO: 267): |
| | | 10 μl Block AB |
| | | 1 μl EP-1 (25 pmol/μl) (SEQ ID NO: 263) |
| | | 1 μl EP-5 (25 pmol/μl) (SEQ ID NO: 267) |
| | | 10 μl 10× pfu buffer |
| | | 2.5 mM dNTP |
| | | 1 μl PFU enzyme |
| | | 74.5 μl H₂O |
| | | Σ 100 μl |
| | | Run 30-cycle PCR, use annealing temperature based on oligos EP- |
| | | 1 (SEQ ID NO: 263) and EP-5 (SEQ ID NO: 267). |
| | w. | Run PCR product out on agarose gel, gel purify, elute in 8 μl. |
| | x. | Clone EPO 3 PCR product into pCRblunt: |
| | | 1 μl pCRblunt vector (Invitrogen, 25 ng/μl) |
| | | 4 μl EPO 3 insert |
| | | 1 μl 10× T4 DNA ligase buffer |
| | | 1 μl T4 DNA ligase |
| | | 3 μl H₂O |
| | | Σ 10 μl |
| | | Incubate 15° C., 2 h to O/N. |
| | | Transform TOP10 cells with 2 μl of ligation. |
| | | Plate on KANAMYCIN plates. |
| | y. | Screen colonies with T7 (SEQ ID NO: 276) and M13r (SEQ ID |
| | | NO: 277) oligos. |
| | z. | Make spin prep of 3 positives |
| | aa. | Sequence 3 positives. |
| | bb. | Make glycerol stock of favorite. |
| 4. | For EPO 4: | |
| | a. | Set up PCR reaction for blocks: |
| | BLOCK A: | |
| | | 1 μl EPO 1 DNA |
| | | 1 μl EP-1 oligo (25 pmol/μl) (SEQ ID NO: 263) |
| | | 1 μl EP-11 oligo (25 pmol/μl) (SEQ ID NO: 273) |
| | | 10 μl 10× pfu buffer |
| | | 2.5 mM dNTP |
| | | 1 μl PFU enzyme |
| | | 83.5 μl H₂O |
| | | Σ 100 μl |
| | BLOCK B: | |
| | | 1 μl EPO 1 DNA |
| | | 1 μl EP-10 oligo (25 pmol/μl) (SEQ ID NO: 272) |
| | | 1 μl EP-5 oligo (25 pmol/μl) (SEQ ID NO: 267) |
| | | 10 μl 10× pfu buffer |
| | | 2.5 mM dNTP |
| | | 1 μl PFU enzyme |
| | | 83.5 μl H₂O |
| | | Σ 100 μl |
| | | Check T_{M} for oligos and run 30-cycle PCR at appropriate |
| | | temperature. |
| | cc. | Run PCR products out on a gel. Gel purify, elute in 8 μl (or |
| | | accordingly based on intensities). |
| | dd. | Set up Gene-Synthesis reaction to put together Block A and Block B: |
| | | 4 μl Block A |
| | | 4 μl Block B |
| | | 5 μl 10× pfu buf |
| | | 2 μl dNTP |
| | | 1 μl PFU |
| | | 34 μl H₂O |
| | | Σ 50 μl |
| | | Run GeneSyn (5 + 10) cycle, use annealing temperature |
| | | based on overlap region (ie. EP-10, EP-11). |
| | ee. | Amplify Block AB (entire EPO 4) with oligos EP-1 (SEQ ID NO: 263) |
| | | and EP-5 (SEQ ID NO: 267): |
| | | 10 μl Block AB |
| | | 1 μl EP-1 (25 pmol/μl) (SEQ ID NO: 263) |
| | | 1 μl EP-5 (25 pmol/μl) (SEQ ID NO: 267) |
| | | 10 μl 10× pfu buffer |
| | | 2.5 mM dNTP |
| | | 1 μl PFU enzyme |
| | | 74.5 μl H₂O |
| | | Σ 100 μl |
| | | Run 30-cycle PCR, use annealing temperature based on oligos EP- |
| | | 1 and EP-5. |
| | ff. | Run PCR product out on agarose gel, gel purify, elute in 8 μl. |
| | gg. | Clone EPO 4 PCR product into pCRblunt: |
| | | 1 μl pCRblunt vector (Invitrogen, 25 ng/μl) |
| | | 4 μl EPO 4 insert |
| | | 1 μl 10× T4 DNA ligase buffer |
| | | 1 μl T4 DNA ligase |
| | | 3 μl H₂O |
| | | Σ 10 μl |
| | | Incubate 15° C., 2 h to O/N. |
| | | Transform TOP10 cells with 2 μl of ligation. |
| | | Plate on KANAMYCIN plates. |
| | hh. | Screen colonies with T7 (SEQ ID NO: 276) and M13r (SEQ ID |
| | | NO: 277) oligos. |
| | ii. | Make spin prep of 3 positives |
| | jj. | Sequence 3 positives. |
| | kk. | Make glycerol stock of favorite. |
| 5. | For EPO 5: | |
| | a. | Set up PCR reaction for blocks: |
| | BLOCK A: | |
| | | 1 μl EPO 1 DNA |
| | | 1 μl EP-1 oligo (25 pmol/μl) (SEQ ID NO: 263) |
| | | 1 μl EP-13 oligo (25 pmol/μl) (SEQ ID NO: 275) |
| | | 10 μl 10× pfu buffer |
| | | 2.5 mM dNTP |
| | | 1 μl PFU enzyme |
| | | 83.5 μl H₂O |
| | | Σ 100 μl |
| | BLOCK B: | |
| | | 1 μl EPO 1 DNA |
| | | 1 μl EP-12 oligo (25 pmol/μl) (SEQ ID NO: 274) |
| | | 1 μl EP-5 oligo (25 pmol/μl) (SEQ ID NO: 267) |
| | | 10 μl 10× pfu buffer |
| | | 2.5 mM dNTP |
| | | 1 μl PFU enzyme |
| | | 83.5 μl H₂O |
| | | Σ 100 μl |
| | | Check T_{M} for oligos and run 30-cycle PCR at appropriate |
| | | temperature. |
| | ll. | Run PCR products out on a gel. Gel purify, elute in 8 μl (or |
| | | accordingly based on intensities). |
| | mm. | Set up Gene-Synthesis reaction to put together Block A and Block |
| | | B: |
| | | 4 μl Block A |
| | | 4 μl Block B |
| | | 5 μl 10× pfu buf |
| | | 2 μl dNTP |
| | | 1 μl PFU |
| | | 34 μl H₂O |
| | | Σ 50 μl |
| | | Run GeneSyn (5 + 10) cycle, use annealing temperature |
| | | based on overlap region (ie. EP-12, EP-13). |
| | nn. | Amplify Block AB (entire EPO 5) with oligos EP-1 (SEQ ID NO: 263) |
| | | and EP-5 (SEQ ID NO: 267): |
| | | 10 μl Block AB |
| | | 1 μl EP-1 (25 pmol/μl) (SEQ ID NO: 263) |
| | | 1 μl EP-5 (25 pmol/μl) (SEQ ID NO: 267) |
| | | 10 μl 10× pfu buffer |
| | | 2.5 mM dNTP |
| | | 1 μl PFU enzyme |
| | | 74.5 μl H₂O |
| | | Σ 100 μl |
| | | Run 30-cycle PCR, use annealing temperature based on oligos EP- |
| | | 1 (SEQ ID NO: 263) and EP-5 (SEQ ID NO: 267). |
| | oo. | Run PCR product out on agarose gel, gel purify, elute in 8 μl. |
| | pp. | Clone EPO 5 PCR product into pCRblunt: |
| | | 1 μl pCRblunt vector (Invitrogen, 25 ng/μl) |
| | | 4 μl EPO 4 insert |
| | | 1 μl 10× T4 DNA ligase buffer |
| | | 1 μl T4 DNA ligase |
| | | 3 μl H₂O |
| | | Σ 10 μl |
| | | Incubate 15° C., 2 h to O/N. |
| | | Transform TOP10 cells with 2 μl of ligation. |
| | | Plate on KANAMYCIN plates. |
| | qq. | Screen colonies with T7 (SEQ ID NO: 276) and M13r (SEQ ID |
| | | NO: 277) oligos. |
| | rr. | Make spin prep of 3 positives |
| | ss. | Sequence 3 positives. |
| | tt. | Make glycerol stock of favorite. |

In addition to demonstrating the reduced immunogenic potential of modified peptides, polypeptides, proteins, and/or antibodies of the invention, it is imperative to show that the modified molecules retain the desired biological activity. To this end, modified erythropoietin molecules of the present invention are tested as follows to determine that they are indeed bioactive and retain their biological function. The structural modeling analysis described herein indicates that none of the proposed substitutions should have a significant impact on biological activity of the modified erythropoietin molecules of the present invention.

For this purpose, small quantity of modified erythropoietin protein is generated according to the methods described in Example 2. The modified protein is then tested for its ability to induce a biological response using the human erythroleukemia cell line TF-1 that expresses the erythropoietin-receptor and is dependant on either IL-3, GM-CSF or EPO for its growth (Hammerling, U., et al., J. Pharma. Biomed. Anal. 12:1455-69 (1996)). A cell-based proliferation assay using this factor-dependant cell line is used as the industry standard for measurement of in vitro biological activity of erythropoietin and mutants thereof (Kitamura, T., et al., J. Cell Physiol. 140:323 (1989)). The advantage of this assay is that it is an extremely sensitive assay that can detect very small amounts of biologically active protein. An additional advantage of this assay is that unpurified supernatants from cell cultures expressing modified erythropoietin molecules of the present invention may be used for testing biological activity rather than employing extensive purification methods to obtain pure protein. The activity of each of the analog proteins will be compared to that of wild type protein and necessary quantification can be done using commercially available ELISA kits.

The above constructs are then used to subclone the appropriate inserts encoding Modified EPO Constructs 1-5 into bacterial and eukaryotic expression vectors using methods that are well-known in the art. Protein is expressed from bacterial and eukaryotic expression vectors according to methods well-known in the art.

### Example 4

### Additional Bioactivity of EPO Analogs

Assays for Peptide-Binding to Purified HLA Molecules

We measured binding affinity of each peptide used in the study against 15 different MHC class II molecules including: DRB1*0101, DRB1*1501, DRB1*0301, DRB1*0401, DRB1*0404, DRB1*0405, DRB1*0701, DRB1*0802, DRB1*0901, DRB1*1101, DRB1*1302, DRB3*0101, DRB4*0101, DRB5*0101 and DQB1*0301. These class II molecules were chosen to be representative of most common HLA class II antigens worldwide. Specifically, the DRB1*0101, DRB1*1501, DRB1*0301, DRB1*0401, DRB1*1101, DRB5*0101, DRB4*0101 and DRB3*0101 allelic forms were chosen as each represents the most prevalent molecule of the DR1, DR2, DR3, DR4, DR11, DR51, DR53, and DR52a antigens respectively. In the case of the DR7 antigen only the DRB1*0701 allele was included in our panel since DRB1*0701 and DRB1*0702 vary at only one position, which is outside the binding groove. In the case of the DR4 antigen in addition to DRB1*0401, the DRB1:0405 allele was also studied because it's high prevalence in Asian populations. For DR8, the DRB1*0802 allele was chosen since it is the dominant allele in the majority population and DRB1*0802 and DRB1*0803 have nearly identical binding specificity's. The DRB1*0901 allele was chosen as representative of the DR9 antigen as most variants allele are mostly associated with silent mutations. The DRB1*1201 allele was arbitrarily to represent DR12 because DRB1*1201 and DRB1*1202 are evenly distributed. These alleles differ at position 67, which is not in the region of the binding groove and hence would not be predicted to strongly impact peptide binding. Finally, the DRB1*1302 allele was chosen to represent DR13 because this allele is slightly more prevalent than DRB1*1301.

The bioactivity of modified EPO proteins were measured in a cell proliferation assay using the human erythroleukemic cell line, designated TF-1, which is dependent on GM-CSF, IL-3 and EPO for growth (Hammerling, et al., (1996) J. Pharm. Biomed. Anal. 11, 1455-69; Kitamura, et al., (1989) J. Cell Physiol. 140, 323-34). The TF-1 cells were cultured in 10 ng/ml of recombinant human GM-CSF. On the day prior to performing the assay, the cells were washed and resuspended in RPMI-1640 media containing no growth factors. 3×10⁴ of these cells were plated in 96-well flat bottom plates and incubated with various dilutions of wild-type EPO protein or concentrated supernates from cells transfected with analog proteins. The cells were incubated at 37° C. for 96 hr and pulsed with 2 μCi of tritiated thymidine for 12-16 hr. The following day, the plates were harvested on to filter mats and radioactivity counted using a Topcount Instrument (Packard Instruments). To confirm the specificity of the bioassay, the EPO specific response was blocked by preincubation of the supernates or wild-type EPO with a polyclonal anti-EPO antibody (10 μg/ml) for 30 minutes. As a toxicity control, we verified that GM-CSF induced proliferation was not blocked using the same anti-EPO antibodies.

Antigenicity Analysis of EPO Epitope and EPO Epitope Analogs

Antigenicity analysis of all 36 EPO peptides was completed using EPO specific HTL lines. The HTL specific lines were tested for their ability to recognize PBMC pulsed with each of 36 wild type EPO peptides individually. IFN-γ production was measured using the ELISPOT; the response generated against PBMC pulsed with an irrelevant peptide was tested as a negative control. Epitope analogs were tested similarly to determine if reduction in HLA-DR binding capacity was associated with reduction in HTL response. The magnitude of responses relative to those observed with wild type EPO peptides was scored as +++ (70-90%), ++ (40-70%), + (10-40%) and − (<10%). The response to an epitope analog peptide in a single donor was considered disrupted if the analog scored as against the “−” or “+”.

Immunogenicity of Analog Peptides and Modified EPO Protein

CD4+ T cells isolated by selection using antibody-coated magnetic beads (Miltenyi Biotec, Auburn, Calif.) from normal donor cells were stimulated in vitro with GM-CSF and IL-4 induced dendritic cells that had been pulsed with either the two wild type EPO epitopes C1 (EPO 101-115 and EPO 136-150) in the form of synthetic peptides or EPO epitope analog combinations C2 (L102P and S146D), C3 (T107D and S146D) C4 (L102G, T107D and S146D) and C5 (L102S, T107D and S146D). These synthetic peptides can be found, for example, in Tables 10A and 10B. For testing immunogenicity of proteins, dendritic cells were pulsed with culture supernates from DNA constructs transfected with either wild-type EPO or modified EPO proteins G3 and G4. Following two in vitro restimulations with peptide or protein pulsed PBMC, cultures were tested for peptide specific responses using the IFN-γ based ELISPOT assay. Five different donors, whose CD4 T cells had responded to EPO, were tested. The data are expressed as the total number of positive cultures (using the positive ELISPOT criteria described above)/number of cultures tested, as a measure of frequency of response and as number of net SFC/5×10⁴ effector cells as a measure of response of magnitude of response.

HLA Class II Binding Capacity of Candidate Proteins

Overlapping 15 amino acid synthetic peptides spanning the entire sequence of calcitonin, human EPO, hGH, human insulin and human IFN [symbol] were tested for binding to a panel of 15 HLA-DR molecules (supplemental data). We found that most of the peptides bound one or more HLA-DR molecules; only 12% of the peptides did not bind any of the HLA-DR molecules tested. These data suggest that generation of proteins completely devoid of immunogenic potential maybe an unrealistic goal (supplemental data). We therefore reasoned that a more practical alternative for reducing immunogenicity was to identify degenerate immunodominant epitopes and to modify them accordingly.

To illustrate the relation between immunodominance and degenerate binding twenty-nine known HLA-DR epitopes were synthesized and tested for binding to 15 different HLA class II molecules. The majority of the reported restriction events 32/48 (67%) were associated with degenerate binding, defined as the capacity to bind 40% (6/15) of the HLA-DR molecules tested.

The degenerate binding regions from the 5 therapeutic proteins insulin, calcitonin, IFNβ, hGH and EPO is shown in Table I. While insulin and calcitonin did not contain any degenerate binding peptides, several highly degenerate regions were identified within EPO, hGH and IFNβ. Four main degenerate binding regions were identified for hGH between residues 8-22, 71-106, 134-147, and 155-169. Each degenerate region contained either one or more overlapping peptides. In the case of IFN [symbol] , five degenerate binding regions were identified, between residues 6-20, 16-30, 56-80, 111-140 and 136-166.

Four main degenerate binding regions were identified in EPO. The first was located between residues 46-65 (binding a maximum of 8/15 molecules; peptide 51-65), the second region was around 66-80 (binding a maximum of 7/15 molecules, peptide 66-80). A third region was located around residues 91-120 (binding a maximum of 11/15 molecules; peptide 101-116). Lastly a fourth region was found between residues 126-155, in this case a maximum of 11/15 molecules were bound by peptide 136-150. In conclusion, degenerate regions were identified for 3/5 proteins analyzed.

Immunogenicity of Wild-Type EPO Protein and Antigenicity of EPO Peptides In Vitro

We used EPO as a model protein and examined its in vitro immunogenicity using human PBMC obtained from normal donors. For these in vitro studies, peripheral blood mononuclear cells were obtained from seven normal donors with a diverse set of MHC haplotypes. EPO-specific HTL responses were induced in 5/7 (71%) of the donors tested (Table II). These data clearly demonstrate the immunogenicity of EPO across various HLA-DR types.

To precisely identify the epitopes recognized, HTL lines from 5 donors were tested using our panel of 36 overlapping EPO peptides. The magnitude of response obtained against each peptide and represented as cumulative net SFC against each individual peptide in all the 5 donors were compared to the number of HLA-DR molecules bound for each individual peptide. Using these data, we mapped two highly immunogenic regions to residues 91-120, containing 3 overlapping peptides 91-105, 96-110 and 101-115, and 126-155, containing 2 overlapping peptides 131-145 and 136-150. While some responses in 2/5 donors were obtained against EPO peptides 51, 68, 86, 106, the magnitude of these responses was significantly lower than those obtained to regions 91-120 and 126-155.

The frequency of response by different donors against EPO peptides was examined. The highest frequency of response (80-100%) was detected against peptides corresponding to EPO position numbers 91, 101 and 136, while lower frequency of response were obtained against several other peptides. Thus, the most degenerate EPO peptides in the regions (91-120) and (126-155) contained the immunodominant epitopes.

Epitope Analogs with Reduced HLA Degeneracy and Antigenicity

We selected the most immunogenic and HLA-DR degenerate EPO peptides, designated as EPO 101 and EPO 136 for further analysis. A total of 100 different analogs of the two wild type peptides EPO 101 and 136 were tested for binding to the 15 HLA-DR molecules. Twelve analogs of the EPO 101 epitope and nine analogs of the EPO 136 were identified with ≧10 fold reduction in binding affinity for at least 5 HLA-DR molecules (Table III a and b). Analogs with the most significant reduction in binding capacities were double substitution analogs. For example, the binding affinity of the GSRSLTDLLRALGAQ and GGRSLTDLLRALGAQ double analogs was reduced by 10 fold or greater for 13/15 and 14/15 HLA-DR respectively. The binding affinity of the corresponding single analogs was reduced for 7/15 or 8/15 HLA-DR molecules. A significant reduction in the number of HLA-DR bound at the 1000 nM level was also observed for the GSRSLTDLLRALGAQ and GGRSLTDLLRALGAQ analogs, binding was reduced to 2/15 or 3/15 HLA-DR molecules respectively. The corresponding single analog bound to 8/15 or 5/15 HLA-DR molecules. Similar results were obtained with the double analog DTFRKDFRVYDNFLR of the EPO 136 epitope (Table IIIb) where the binding capacity was reduced by 10 fold or greater in 11/15 molecules. These results compare favorably with those from corresponding single analogs where the binding capacity was reduced in 8/15 or 5/15 molecules. In fact, the double analog bound to only 3/15 MHC molecules, compared to the corresponding single analogs, which bound 9/15 or 7/15 molecules.

The antigenicity of these epitope analogs was assessed using HTL lines, specific for the whole EPO protein, derived from four donors (Table IV). Single amino acid substitutions associated with the greatest reductions in binding affinity were recognized least; for example for peptides GLRSLTDLLRALGAQ, GPRSLTTLLRALGAQ, DTFRKLFRVYDNFLR and DTFRKDFRVYSNFLR responses were disrupted by more than 60% in more than 75% of the donors. As predicted, the double analogs GSRSLTDLLRALGAQ, GGRSLTDLLRALGAQ and DTFRKDFRVYDNFLR were recognized to a lower level, with decreases in magnitude ≧90% in ≧75% of donors tested.

Molecular Modeling of EPO Analogs

We analyzed which domains and residues may be important for EPO bioactivity. Our analysis indicated that 70% of substitutions for amino acids located within the EPO receptor contact sites, at positions R103, 5104, R143 and N147 result in decreased bioactivity compared with just 6% for substitutions of amino acids outside these contact sites. The amino acid substitutions in the EPO epitopes, EPO 101-115 (GARSLTTLLRALGAQ) and EPO 136-150 (DTFRKLFGVYSNFLR), at positions L102, R103, S104, L105, T107, L141, R143, V144, S146 and N147 were analyzed further. In the primary mammalian sequences, little conservation was seen in S104. Residues L105, T107, V144 and S146 were variable although substitutions at these positions were generally quite conservative. Moderate conservation was seen for R103 and T107. Comparison of structural neighbors for the five residues in peptide EPO 101-115 showed that L102 was very well conserved with most residues in the structural neighbors preserving the aliphatic nonpolar nature of this position. For experimental variants within the peptide EPO 136-150 (DTFRKLFGVYSNFLR), all residues except V144 were well conserved in the structural neighbors.

The experimental EPO variants were modeled using the EPO structure 1EER:A as the template structure for model building. With the exception of the substitution of P at L102 (root square mean deviation of 0.55 Å) the proposed experimental substitutions had little effect on the gross predicted structures of the EPO variant proteins with root mean square deviations for the main backbone of 0.14 Å or less.

Considering these data together, (Table IV) we determined that experimental substitutions within EPO epitopes EPO 101-115 and EPO136-150, with the possible exceptions of the L→P102 analog and N→D147 which is located within the receptor contact site, could result in EPO variants that retained wild-type bioactivity in vivo (Table IV).

Immunogenicity of EPO 101 and EPO 136 Epitope Analogs with Reduced Binding Capacities

To confirm that the EPO epitope analogs with disrupted HLA-DR binding affinity were less immunogenic, we utilized PBMC from 5 donors that had responded previously wild-type EPO and against the wild type EPO epitopes EPO 101 and EPO 136 and tested peptide combinations using the primary in vitro immunogenicity assay. In each case, we included one analog from the EPO 101 epitope, and one from of the EPO 136 epitope. These peptides were pooled in equimolar concentrations and tested; the wild-type epitopes EPO 101 and EPO 136 were also pooled at equimolar concentrations and used as positive controls. For each combination, the magnitude of responses net SFC and frequency of response in 10 individual cultures from each donor were recorded. The data obtained are shown in FIG. 12.

The analog epitope combinations were significantly less immunogenic. For example, the mean response against the wild-type C1 combination was 319 SFC/5×10⁴ effector cells, while the mean values for the peptide combinations C2, C3, C4 and C5 were 20, 86, 45, and 29 SFC/5×10⁴ effector cells, respectively. In all cases, the differences between the response induced using the analog and the wild-type epitopes were highly significant (p<0.005). With respect to the frequency of responses, the wild-type C1 peptide combination yielded 36/50 positive cultures whereas the analog C2, C3, C4 and C5 yielded 9/50, 12/50, 6/50 and 14/50 positive cultures respectively. Again, these differences are highly significant (p<0.005).

Expression and Production of Modified EPO Proteins

DNA constructs encoding EPO variants carrying four different amino acid substitutions designated G2 (L102P, S146D), G3 (T107D, S146D), G4 (L102G, T107D, S146D) and G5 (L102S, T107D, S146D) were generated. Following de novo synthesis of the parent EPO construct, specific substitutions were introduced using overlapping complementary oligonucleotides encoding the analog mutations in a PCR assay. Blocks with 20 nucleotide overlap were generated for each analog and annealed together and extended in a gene synthesis reaction (94° C., 30 sec 58° C., 30 sec; 72° C., 1 min for 5 cycles; 94° C., 30 sec, 72° C., 1 min for 10 cycles) using the proof-reading polymerase Pfu (Stratagene, La Jolla, Calif.). The extended blocks were amplified by PCR (94° C., 30 sec; 58° C., 30 sec; 72° C., 1 min; 30 cycles) to synthesize full-length EPO analogs. Gel purified EPO analog PCR products were cloned into pCRblunt (Invitrogen, Carlsbad, Calif.) and confirmed by sequence analysis. The restriction sites HindIII and BamH1 were engineered into the 5′ and 3′ ends, respectively, of each construct, which was then subcloned into a mammalian expression vector.

For expression of the analoged EPO sequences, HEK-293 cells were plated at a density of 2×10⁶ cells/100 mm poly-lysine coated cell-culture dish in complete RPMI media. The following day, cells were washed with RPMI without any antibiotics or serum and transfected with 9 μg of plasmid DNA complexed to GenePorter liposome formulation (Gene Therapy Systems, San Diego, Calif.). Specifically, plasmid DNA and 45 μl liposome were each diluted into 0.5 ml media and then combined. After allowing DNA to complex to the liposomes for 20 min at RT, the liposome DNA mixture was added directly to the washed cells. Control wells were included, which did not contain any DNA. After adding an additional 4 ml of complete media, the cells were incubated at 37° C. and 5% CO₂ for 3 hr. Following incubation, cells were washed once with complete RPMI media and incubated for 72 hrs to allow for EPO expression. Cell supernates were concentrated ˜6 fold by centrifugation at 2000 rpm using an Allegra 6KR centrifuge (Beckman Coulter, Fullerton, Calif.) with an Amicon Centriprep (YM-10) ultracentrifugation device (Millipore, Billerica, Mass.).

Expression, Production and Bioactivity of Modified EPO Protein

Modified EPO proteins containing substitutions corresponding to epitope analogs (G2, G3, G4 and G5) were produced. When expression levels were tested by ELISA and by western blot, the modified proteins G3 G4 and G5 were easily detected while G2 was not. Two different EPO antibodies were used in this ELISA assay to ensure that the lack of detection of G2 was not due to the lack of expression but due to loss of reactivity with a particular antibody. We therefore believe that our inability to detect the G2 form of the protein reflects a gross structural alteration associated with the L→P substitution at position 102. The amount of modified EPO proteins contained in the culture supernatants was quantitated using recombinant EPO protein. The levels varied from 165 U/ml for G3, 211 U/ml for G4 and 152 U/ml for G5 (data not shown); these differences were not considered to be significant.

Bioactivity analysis of the modified EPO constructs was variable. The modified protein G3 and G4 supernates were bioactive in the same range as that of the commercially available wild-type EPO protein. However, we could not detect any bioactivity for the G5 protein supernatant. Thus the L→S change at position 102 appears to significantly alter functional activity.

Immunogenicity of Bioactive Modified EPO Proteins

The wild-type EPO and the modified EPO proteins, G3 and G4, were tested to assess their immunogenicity using PBMC from the 5 donors known to be capable of responding to wild type EPO. The data was evaluated in terms of both magnitude and frequency of response. The immunogenicity of proteins G3 and G4 was lower than that of wild-type EPO protein, a mean response of 44 SFC/5×10⁴ effector cells for the wild-type protein versus mean response of 6 and 9 SFC/5×10⁴ effector cells for modified proteins G3 and G4 (p<0.001). The frequency of cultures generated using the modified EPO was also lower, with 27/40 positive cultures for wild-type protein versus 9/40 and 8/40 positive cultures for the G3 and G4, respectively. As expected, supernates from untransfected cells were not immunogenic. In conclusion, our data demonstrate that the intrinsic immunogenicity of EPO can be reduced by modification of immunodominant HTL epitopes while retaining bioactivity.

We developed an integrated approach to reduce the intrinsic immunogenicity of therapeutic protein drugs. The approach is based on the identification of immunodominant epitopes and their modification to reduce binding affinity to HLA-DR molecules to levels below those associated with naturally-occurring HTL epitopes. Our approach is based on quantitative objective measures, specifically the determination of HLA-DR binding affinity coupled with confirmation of these epitopes by direct immunogenicity testing. These quantitative measurements help in determining the overall intrinsic immunogenic potential of a protein. In addition these measurements provide an empirical value against which subsequent modifications or reduction in immunogenicity can be measured, and thereby, help in determining the success of the approach. We are aware that, immunogenicity of a protein is dependent not only on intrinsic immunogenic potential encoded in its HLA binding characteristics but also on several extrinsic factors, such as formulation, dose and/or route of administration. Although our approach does not address this issue, it does provide a means to objectively assess the intrinsic immunogenicity potential of a protein and ways to reduce this intrinsic immunogenicity. In summary, our approach is a broadly applicable tool as a first fundamental step in determining the immunogenic potential of therapeutic proteins and judging whether any modifications are required or not.

Using this approach, we produced two modified EPO analogs with reduced levels of immunogenicity, tested using an in vitro primary immunogenicity assay with human T cells. In initial experiments the binding capacity of 105 overlapping synthetic peptides spanning the sequences from EPO, hGH, insulin, calcitonin and IFNβ were evaluated to determine the predicted immunogenic potential of these drugs in a population of diverse ethnic backgrounds. These 105 peptides were tested for binding to a panel of 15 HLA molecules representative of the main HLA-DR antigens worldwide (Southwood et al., (1998) J. Immunol. 160, 3363-73, and supplemental data). To the best of our knowledge, this is the most comprehensive analysis of this type completed when HLA polymorphism is considered.

The results of the binding analysis showed that 88% of the peptides bound to one or more of the HLA molecules tested, suggesting that complete elimination of HLA-DR binding capacity is an unrealistic approach and that generation of proteins completely devoid of any immunogenic potential not be a very practical goal. The high number of substitutions required, would be a very laborious process and would generate a new protein, with high non-self content, and thereby intrinsic immunogenic potential, and poorly predictable biological activity. However, the binding analysis provided us a means to identify immunodominant epitopes with degenerate binding patterns. Modification of these epitopes to reduce immunogenicity, might be a realistic and practical approach.

We tested our approach using 5 different therapeutic proteins for which evidence of immunogenicity exists in humans, namely hGH, calcitonin, insulin, IFNβ and EPO (Tangri, et al., (2002) Curr. Med. Chem. 24, 2191-99; Kozono, et al., (1993) Endocrinology 131, 2885-90; Casadevall, et al., (2002) N. Engl. J. Med. 346, 469-75; Deisenhammer, et al., (2001) Neurology 54, 2055-60; Abdul-Ahad, et al., (1997) Cytokines Cell Mol. Ther. 1, 27-32). While insulin and calcitonin did not contain any clear degenerate binding peptides, four, six and four degenerate binding regions were identified within the hGH, IFNβ and EPO proteins respectively. This suggested that the intrinsic binding capacity of these proteins might be reduced with a relatively small number of substitutions. We selected EPO as a model antigen for detailed studies as functional and biological assays for testing this bioactivity of this molecule were available and due to the existence of a large structural database for EPO.

The immunogenicity of the intact EPO protein was evaluated using primary in vitro immunogenicity assays utilizing PBMC from normal healthy blood donors. We used in vitro human primary assays rather than in vivo animal models, since most candidate therapeutic proteins are human proteins and it is predicted that they would be recognized as non-self in most laboratory animals. Moreover, the type of MHC molecules expressed in animal models is associated with a peptide binding specificity different from human MHC, resulting in a different set of epitopes being presented. Finally, we emphasize that as actual binding determinations against HLA-DR molecules representative of the worldwide population was performed, any bias resulting from sampling populations with a narrow ethnic spread was avoided.

Our results demonstrate that immunodominant epitopes correspond to the most degenerate HLA-DR binding regions at least in the case of EPO. These findings support earlier reports that describe a relationship between immunodominance and degenerate binding (Panina-Bordignon, et al., (1989) Eur. J. Immunol. 19, 2237-42; Roche, et al., (1991) J. Immunol. 144, 1849-56; Diepolder, et al., (1997) J. Virol. 8, 6011-19; Ferrari, et al., (1991) J. Clin. Invest. 1, 214-22). Our results therefore indicate that a few immunodominant epitopes with degenerate binding could be targeted for the purpose of reducing the immunogenicity of protein drugs. Introducing as few substitutions as possible is highly desirable, as each substitution introduced could impact the molecule's bioactivity or create a neoepitope, based on introduction of a non-self residue in the sequence of the self-protein.

Several analogs that are associated with significantly reduced binding capacity, antigenicity and immunogenicity were identified. To accomplish this goal, an analoging strategy was developed utilizing D, R and G substitutions at non-anchor positions in addition to avoiding aromatic or hydrophobic residues at the P1 anchor position. This strategy greatly reduced the number of analogs to be generated for each epitope. Because of its general applicability, we anticipate that this strategy might be of use for the generation of other protein antigens with reduced immunogenicity.

In our studies, we utilized structure/function data, sequence conservation and structural modeling analysis to predict the impact of the substitutions on the biological activity of the EPO protein. These studies illustrate how bioinformatic structural analysis can be integrated with biochemical and cellular assays, to design proteins with reduced immunogenicity, while preserving biological and pharmacological function.

When the immunogenicity of the two modified EPO proteins was compared to that of wild-type protein, we found that reduced HLA-DR binding affinity values were associated with significantly reduced immunogenicity. Our results suggest that the immunogenicity of EPO protein can be reduced by modification of the immunodominant epitopes, while it is still possible that such modified protein drugs might still be immunogenic and that immune responses against subdominant epitopes might develop. We predict that such responses will be of lower frequency and/or magnitude since the intrinsic immunogenic potential of the protein has been reduced.

In summary, this is the first demonstration of an integrated and systematic approach for reducing immunogenicity of therapeutic proteins. Our approach utilizes high throughput in vitro biochemical assays to quantitatively measure HLA-DR binding to a representative panel of HLA allele variants in conjunction with cellular immunogenicity assays to identify immunodominant epitopes. Epitope identification was followed by rational modification of these epitopes to reduce their HLA DR binding capacity, verification of bioactivity and reduced immunogenicity of analog proteins. Our findings have potentially important implications from the standpoint of engineering safer protein drugs and provide rationale and support to evaluate the proteins defined according to our procedure in the clinical setting.

The above examples are provided to illustrate the invention but not to limit its scope. Other variants of the invention will be readily apparent to one of ordinary skill in the art. All publications, patents, patent applications, drawings, figures and Sequence Listings cited herein are hereby incorporated by reference for all purposes.

TABLE 1
High Degree of Polymorphism in HLA-DR Molecules

| | | | Phenotypic Frequencies | | | | | |
|---|---|---|---|---|---|---|---|---|
| | Antigen | Representative assay(s) | Cauc. | Blk. | Jpn. | Chn. | Hsp. | Avg. |
| a. DRβ1 gene | DR1 | DRβ1*0101 | 18.5 | 8.4 | 10.7 | 4.5 | 10.1 | 10.4 |
| | DR2 | DRβ1*1501 | 22.0 | 16.5 | 32.2 | 23.9 | 21.9 | 23.3 |
| | DR3 | DRβ1*0301 | 17.7 | 19.5 | 0.4 | 7.3 | 14.4 | 11.9 |
| | DR4 | DRβ1*0401-05 | 23.6 | 6.1 | 40.4 | 21.9 | 29.8 | 24.4 |
| | DR5 | DRβ1*1101/1201 | 19.5 | 23.0 | 17.7 | 35.1 | 23.3 | 23.7 |
| | DR5 | DRβ1*1302 | 23.8 | 20.0 | 24.1 | 16.1 | 24.8 | 21.7 |
| | DR7 | DRβ1*0701 | 26.2 | 11.1 | 1.0 | 15.0 | 16.6 | 14.0 |
| | DR8 | DRβ1*0802 | 5.5 | 10.9 | 25.0 | 10.7 | 23.3 | 15.1 |
| | DR9 | DRβ1*0901 | 3.6 | 4.7 | 24.5 | 19.9 | 6.7 | 11.9 |
| | DR10 | DRβ1*1001 | 3.6 | 1.4 | 1.2 | 3.4 | 3.0 | 2.5 |
| b. DRβ3/4/5 gene | DR51 | DRβ5*0101 | 22.0 | 16.5 | 32.2 | 23.9 | 21.9 | 23.3 |
| | DR53 | DRβ4*0101 | 54.2 | 55.2 | 39.7 | 53.2 | 55.4 | 51.4 |
| | DR52 | DRβ3*0101 | 48.9 | 21.1 | 59.6 | 50.9 | 48.6 | 45.8 |

TABLE 2
Binding of Known DR-Restricted Immunodominant Epitopes

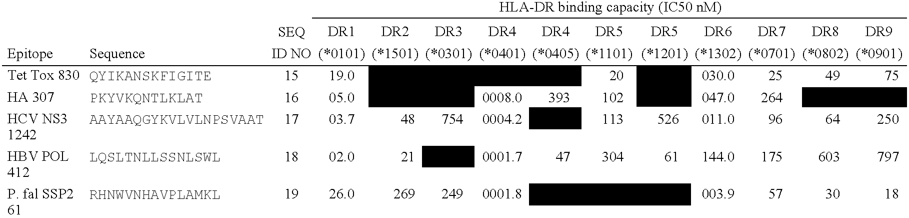

— indicates binding affinity ≧ 20,000 nM.
*Shading indicates IC50 > 1000 nM

TABLE 2
Binding of Known DR-Restricted Immunodominant Epitopes

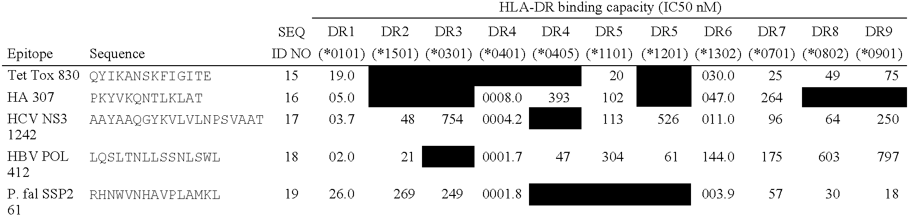

— indicates binding affinity ≧ 20,000 nM.
*Shading indicates IC50 > 1000 nM

TABLE 3
Protein Therapeutics Considered for
Analysis

| | Human Growth | Erythropoietin | | Interferon β | |
|---|---|---|---|---|---|
| PARAMETERS | Hormone (hGH) | (EPO) | Insulin | (IFN β) | Calcitonin |
| Disease | hGH deficiency/ | Anemia | Diabetes | Multiple | Paget's |
| Indication¹ | Metabolic disorders | | | sclerosis | disease/ |
| | | | | | Osteoporosis |
| Immunogenicity² | YES | YES | YES | YES | YES |
| Models/Markers³ | YES | YES | YES | YES | YES |
| Mouse Homology⁴ | 70 | 88 | 93 | 50 | 54 |
| (%) | | | | | |
| Size | 191 | 165 | 51 | 165 | 32 |
| (amino acids) | | | | | |
| Type of molecule | Analog | Self | Self | Analog | Non-self |
| Number of | 2 | 2 | 4 | 0 | 1 |
| Disulfide Bonds | | | | | |
| Delivery Method | Subcutaneous | Subcutaneous | Subcutaneous | Intramuscular | Subcutaneous |
| | | Intravenous | Pulmonary | | Nasal |
| Multimeric | None | None | YES | YES | None |
| Molecular Forms | Reported | Reported | | | Reported |

¹Potential target indications considered for technology validation are those not associated with immunosuppression (e.g., therapeutics for diseases such as cancer, rheumatoid arthritis and AIDS were excluded as candidates)
²Documented immunogenicity in scientific publication(s), package insert information, FDA documents/applications, and/or patent application(s)
³Existence of animal models for in vivo testing of functional activity; Availability of bioactivity markers to determine functional activity using in vitro assays
⁴Percentage of identical amino acid residues between human and mouse proteins

TABLE 4
Efficiency of Predictive Algorithms for
DRB1*0101 Binding

| Sensitivity of | Algorithm Efficiency (no. binders/no. peptides synthesized) | | | |
|---|---|---|---|---|
| Prediction Algorithm (%) | PIC | Propred | MHC Thread | SYFPEITHI |
| 100 | 51% (53/104) | 50% (53/105) | 51% (53/103) | 51% (53/104) |
| 90 | 45% (47/104) | 45% (47/105) | 53% (47/88) | 45% (47/104) |
| 75 | 78% (39/50) | 37% (39/105) | 61% (39/64) | 54% (39/71) |
| 50 | 84% (26/31) | 25% (26/105) | 55% (26/47) | 70% (26/37) |
| 34 | 82% (18/22) | 82% (18/22) | 51% (18/35) | 75% (18/24) |

TABLE 5A
EPO Binding

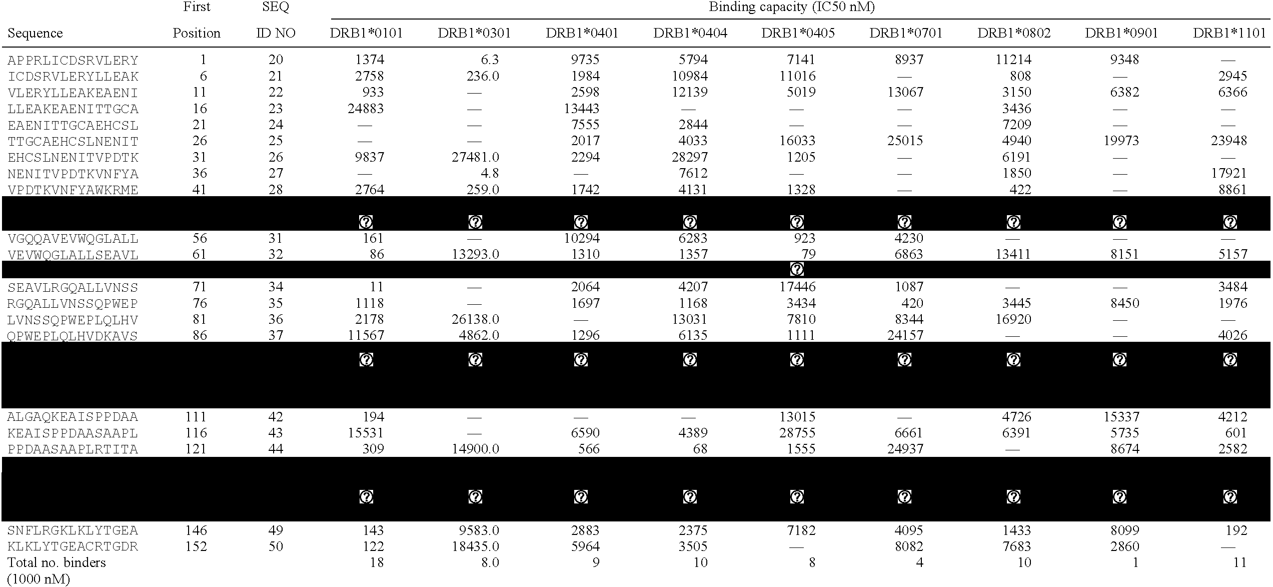

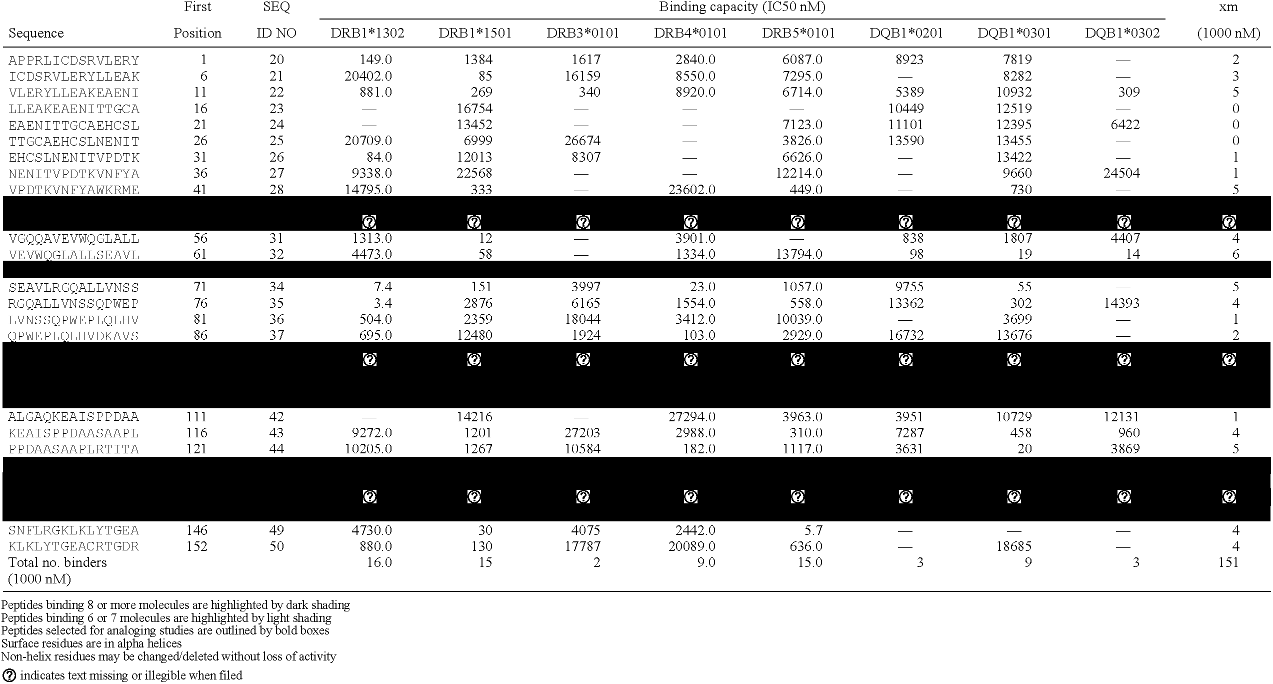

TABLE 5B
Salmon Calcitonin Binding

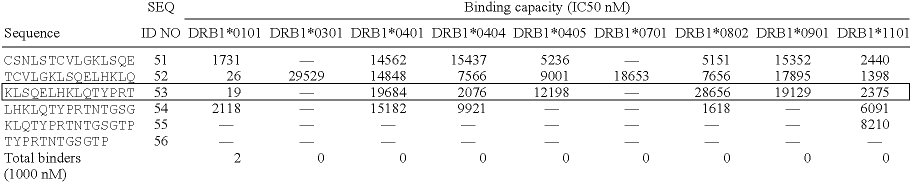

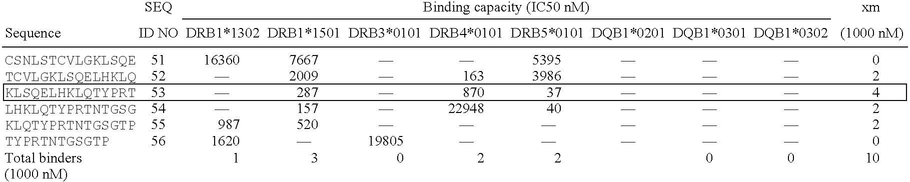

Peptides binding 8 or more molecules are highlighted by dark shading
Peptides binding 6 or 7 molecules are highlighted by light shading
Peptides selected for analoging studies are outlined by bold boxes

TABLE 5C
Human Growth Hormone Binding

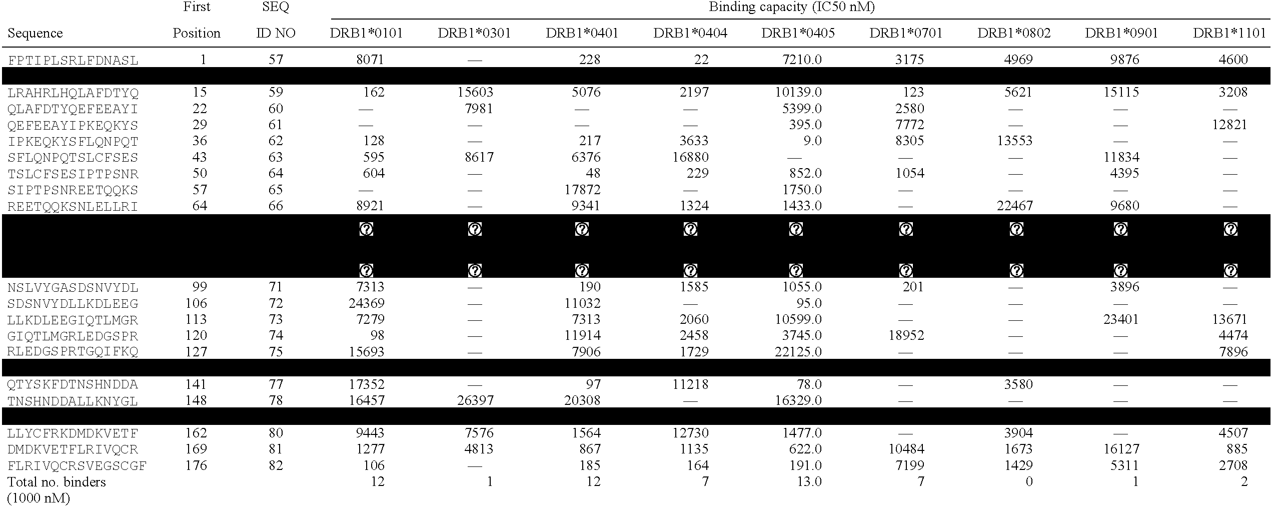

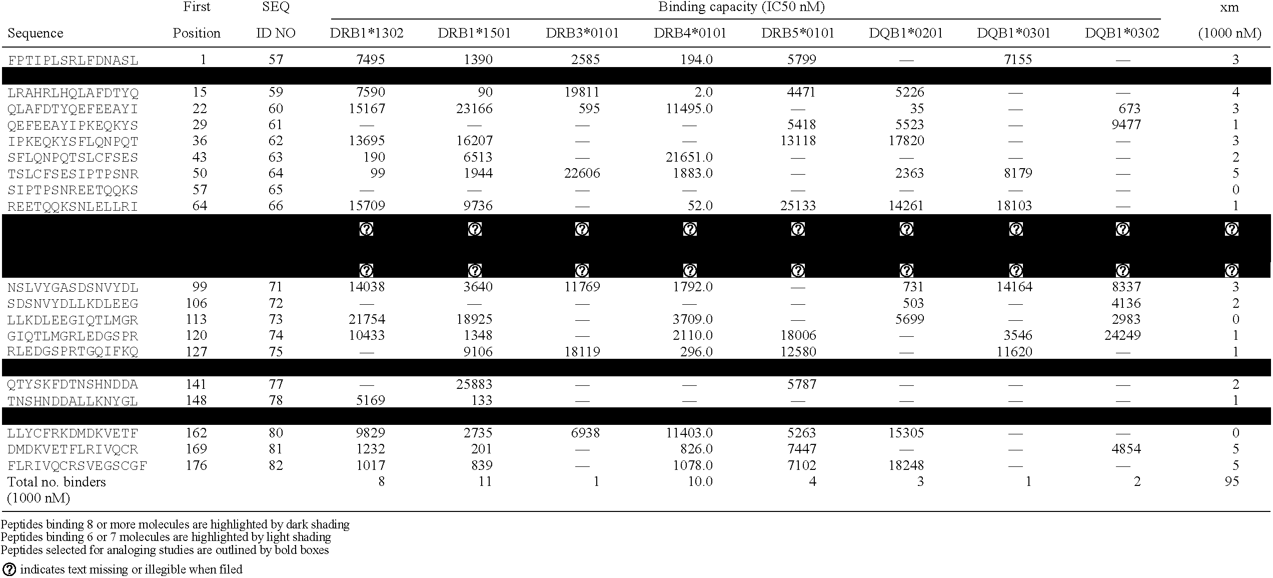

TABLE 5D
IFNb Binding

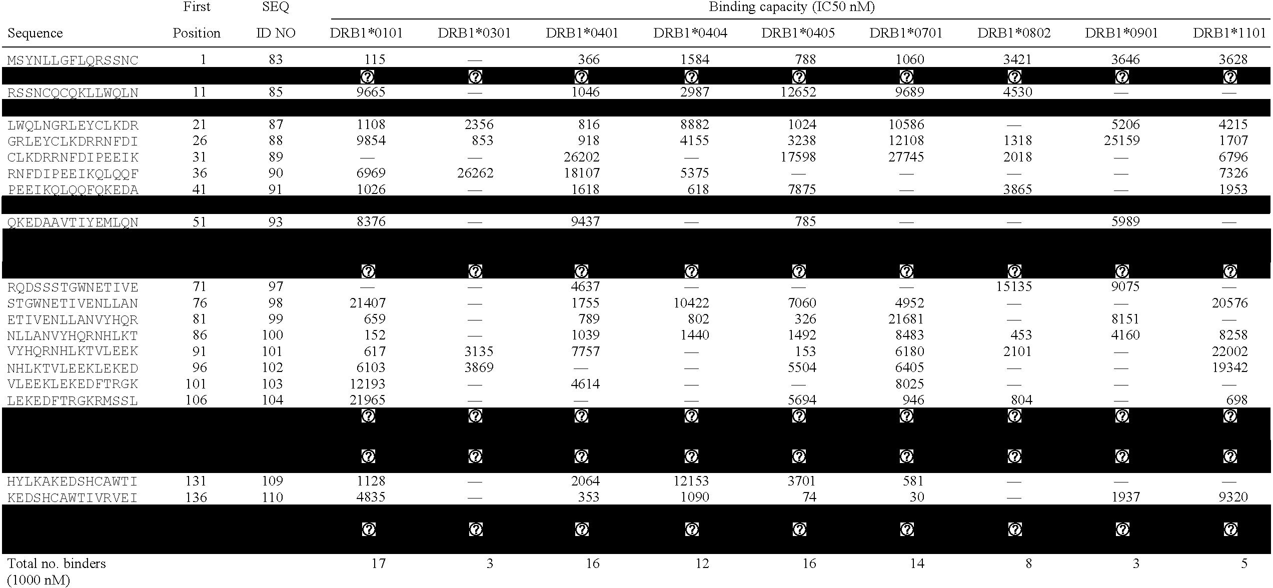

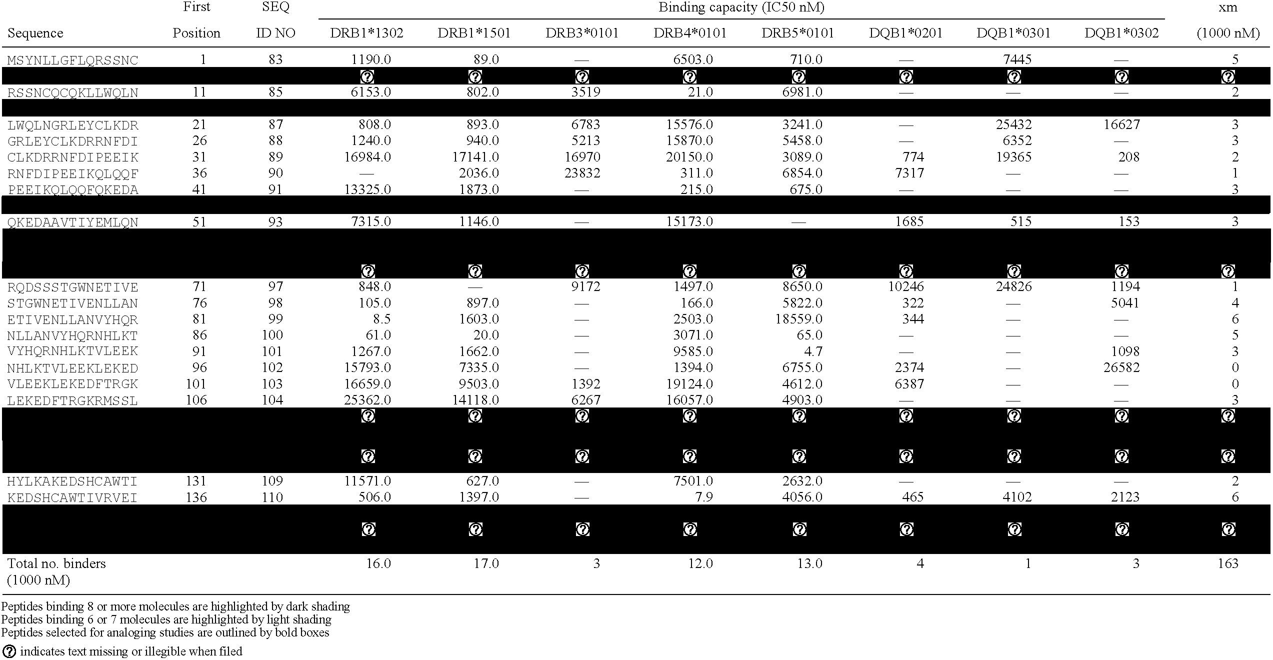

TABLE 5E
Insulin Binding

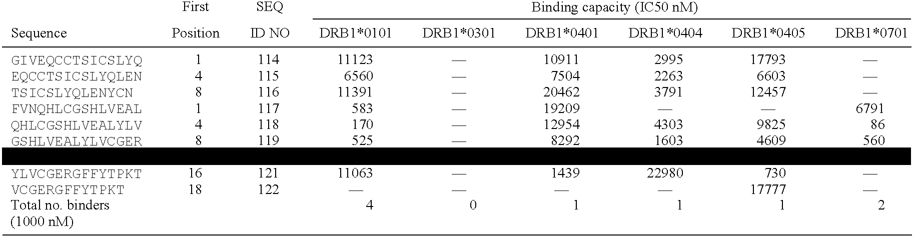

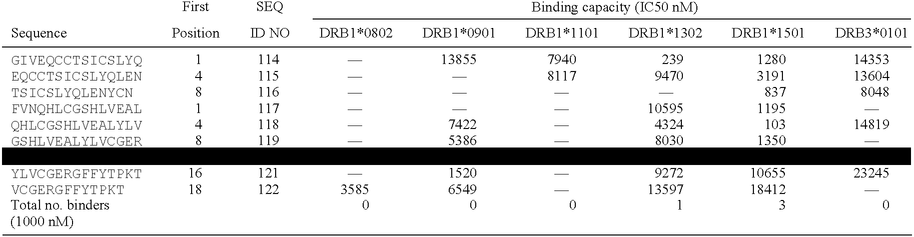

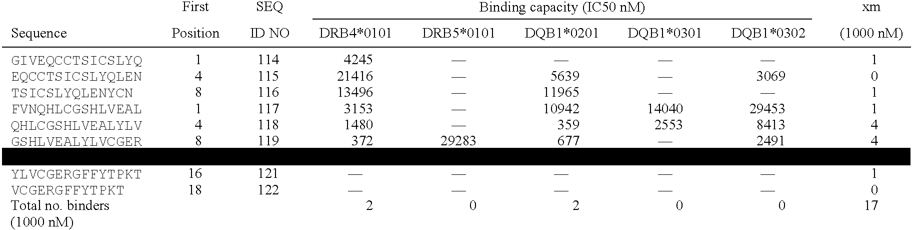

Peptides binding 8 or more molecules are highlighted by dark shading
Peptides binding 6 or 7 molecules are highlighted by light shading
Peptides selected for analoging studies are outlined by bold boxes

TABLE 6
Binding Capacity of Known HLA-DR and DQ Resticted Epitopes

| | SEQ | | | Binding capacity (IC₅₀ nM) | | |
|---|---|---|---|---|---|---|
| Sequence | ID NO | Source | Restriction | DRB1*0101 | DRB1*0301 | DRB1*0401 |
| APYHFDLSGHAFGSMAKKGE | 123 | Lol pl 101-120 | DQ3.1, 3.2 | — | — | 4.4 |
| AVLEDPYILLVSSKVS | 124 | MT 211-226 | DR1 | 12 | — | 182 |
| DALESIMTTKSVSFR | 125 | Rabies virus gp | DR7 | 385 | 10,607 | 20 |
| DIEKKIAKMEKASSVFNVVNS | 126 | Pf CSP 378-398 | DR1, DR4, DR5, DR7 | 17 | 16,432 | 97 |
| DNVLDHLTGRSC | 127 | B. Pertussis 30-41 | DR1 | 455 | — | — |
| DTPYLDITYHFVMQRLPL | 128 | Torp. Ach Rec. 195-212 | DQ1 | 385 | 68 | 160 |
| DYSYLQDSDPDSFQD | 129 | Tyrosinase 448-462 | DR4w4 | — | — | 153 |
| EFVVEFDLPGIKA | 130 | Lysozyme 18K 38-50 | DR4w4 | — | 1773 | 59 |
| ESWGAVWRIDTPDKLTGPFT | 131 | Lol pl 191-210 | DR52a | 2500 | 3177 | — |
| EVWREEAYHAADIKD | 132 | RAG 51-65 | DR1 | 135 | — | 727 |
| FNNFTVSFWLRVPKVSASHLEQY | 133 | TT 947-969 | DR5, DPw2, DPw4 | 417 | — | 93 |
| GDVVAVVDIKEKGKDXWIELK | 134 | Lol pf 171-190 | DR3 | — | 322 | — |
| GYKVLVLNPSVAAT | 135 | HCV.NS3.1248 | DR4, 5w11, 12, 6w19 | 1.4 | 921 | 7.5 |
| IVHATGFKQSSKALQRPVASDFEP | 136 | CML bcr-abl | DR5w11 | 1468 | ND | 1038 |
| LLPLLEKVIGAGKPL | 137 | MT 231-245 | DR2(ab?) | 1667 | — | — |
| NGQIGNDPNRDIL | 138 | TT 1272-1284 | DR52a, c | — | 1875 | — |
| NPVVHFFKNIVTPRTPPPSQ | 139 | Hu MBP 84-103 | DR2b, DR5 | 13 | 15,432 | 1.3 |
| PHHTALRQAILCWGELMTLA | 140 | HBV core 50-69 | DPw4 | 810 | — | 26 |
| PKYVKQNTLKLAT | 141 | Flu HA 307-319 | DR1, DR4, DR5, DR7 | 5.0 | — | 8.0 |
| PLGFFPDHQLDPAFGANSNNPDWDFNP | 142 | HBV Pre S1 21-47 | DQw1 | — | — | — |
| QNILLSNAPLGPQFP | 143 | Tyrosinase 56-70 | DR4w4 | 25 | 6811 | 198 |
| QYIKANSKFIGITE | 144 | TT 830-843 | 1, 2, 3/52a, 4, 5, 7 | 19 | 2320 | 1577 |
| RDTGILDSIGRFFGGDRGAP | 145 | Hu MBP 33-52 | DR5 | — | 1823 | — |
| VDAQGTLSKIFKLGGRDSRS | 146 | Hu MBP 144-163 | DR1, DQ, DP | 25 | 1383 | 1600 |
| WTTCQSIAFPSKTSASIGSL | 147 | PLP 180-199 | DR2a, DR2b | ND | 17,308 | 22 |
| YKTIAYDEEARR | 148 | MT 65 kD 3-13 | DR3 | — | 143 | 4000 |
| YLDPLIRGLLARPAKLQV | 149 | NP7 (TPO Y535-551) | DP2, DQ6 | 1.6 | — | 687 |
| YSGPLKAEIAQRLEDV | 150 | MAT (Y)17-31 | DR1 | 13 | 11,339 | 2567 |
| YTLLQAAPALDKLKLTGDEATGANI | 151 | M leprae 65 kD Y464 | DR2(ab?) | 2.1 | 2050 | 286 |
| Overall hit rate | | | | 17 | 4 | 17 |

| | Binding capacity (IC₅₀ nM) | | | | | | |
|---|---|---|---|---|---|---|---|
| Sequence | DRB1*0404 | DRB1*0405 | DRB1*0701 | DRB1*0802 | DRB1*0901 | DRB1*1101 | DRB1*1302 |
| APYHFDLSGHAFGSMAKKGE | — | 4195 | — | 2708 | 195 | 1000 | — |
| AVLEDPYILLVSSKVS | 538 | 188 | 73 | 9573 | 3444 | 6667 | 8808 |
| DALESIMTTKSVSFR | 50 | 566 | 64 | 1426 | 12,498 | 5000 | 313 |
| DIEKKIAKMEKASSVFNVVNS | 992 | 3386 | 2485 | — | — | 90 | >15,000 |
| DNVLDHLTGRSC | — | >10,000 | — | — | — | 10,000 | — |
| DTPYLDITYHFVMQRLPL | — | 204 | 391 | 555 | 928 | — | 397 |
| DYSYLQDSDPDSFQD | — | — | — | — | — | — | — |
| EFVVEFDLPGIKA | — | >2500 | — | 16,897 | >10,000 | — | >10,000 |
| ESWGAVWRIDTPDKLTGPFT | 333 | 1732 | — | 424 | 9223 | 444 | 3936 |
| EVWREEAYHAADIKD | 16,687 | >2500 | — | — | — | 10,000 | >7500 |
| FNNFTVSFWLRVPKVSASHLEQY | 4545 | 202 | 57 | 581 | 10,859 | 8.3 | 6371 |
| GDVVAVVDIKEKGKDXWIELK | — | >2500 | — | 10,871 | >5000 | — | >5000 |
| GYKVLVLNPSVAAT | 33 | 125 | 126 | 63 | 208 | 75 | 8.6 |
| IVHATGFKQSSKALQRPVASDFEP | ND | 441 | ND | 346 | 1014 | 17 | 89 |
| LLPLLEKVIGAGKPL | 12,500 | 5245 | — | 440 | — | 6687 | — |
| NGQIGNDPNRDIL | — | 4815 | — | — | >10,000 | — | 1040 |
| NPVVHFFKNIVTPRTPPPSQ | 36 | 10 | 87 | 60 | 2627 | 17 | 6298 |
| PHHTALRQAILCWGELMTLA | 1214 | 1698 | 1347 | 210 | 4589 | 602 | 9550 |
| PKYVKQNTLKLAT | — | 393 | 301 | 4017 | 11,752 | 102 | 47 |
| PLGFFPDHQLDPAFGANSNNPDWDFNP | — | 31 | — | — | 15,721 | — | >10,000 |
| QNILLSNAPLGPQFP | 143 | 1214 | 296 | 13,418 | 1286 | 15811 | 5.3 |
| QYIKANSKFIGITE | — | 2384 | 6.6 | 49 | 75 | 21 | 30 |
| RDTGILDSIGRFFGGDRGAP | — | 10,704 | — | 6325 | 8898 | 169 | 828 |
| VDAQGTLSKIFKLGGRDSRS | 314 | 1505 | 1171 | 173 | 6345 | 18 | >5000 |
| WTTCQSIAFPSKTSASIGSL | | 2549 | | 3051 | 1717 | — | 74 |
| YKTIAYDEEARR | — | >2500 | — | — | >10,000 | — | >10,000 |
| YLDPLIRGLLARPAKLQV | 44 | >2500 | — | 165 | — | 101 | >7500 |
| YSGPLKAEIAQRLEDV | — | >2500 | 208 | — | 18,047 | — | 771 |
| YTLLQAAPALDKLKLTGDEATGANI | 1111 | 108 | — | 160 | 1954 | 200 | >5000 |
| Overall hit rate | 9 | 10 | 10 | 12 | 4 | 14 | 10 |

| | Binding capacity (IC₅₀ nM) | | | | | No. of Alleles | | |
|---|---|---|---|---|---|---|---|---|
| Sequence | DRB1*1501 | DRB3*0101 | DRB4*0101 | DRB5*0101 | DQB1*0301 | Bound | Tested | Hit rate |
| APYHFDLSGHAFGSMAKKGE | — | 172 | — | 10,000 | 90 | 5 | 15 | 0.333 |
| AVLEDPYILLVSSKVS | 336 | 5000 | 4206 | 2500 | — | 6 | 15 | 0.400 |
| DALESIMTTKSVSFR | 6021 | — | 69 | — | — | 7 | 15 | 0.467 |
| DIEKKIAKMEKASSVFNVVNS | 1005 | — | — | 1812 | 272 | 5 | 15 | 0.333 |
| DNVLDHLTGRSC | — | 11,111 | — | — | — | 1 | 15 | 0.067 |
| DTPYLDITYHFVMQRLPL | 1406 | 800 | 261 | — | — | 10 | 15 | 0.687 |
| DYSYLQDSDPDSFQD | — | 6589 | — | — | ND | 1 | 14 | 0.071 |
| EFVVEFDLPGIKA | 142 | 14,286 | 4245 | — | — | 2 | 15 | 0.133 |
| ESWGAVWRIDTPDKLTGPFT | 1891 | 19 | 1117 | 2222 | 185 | 5 | 15 | 0.333 |
| EVWREEAYHAADIKD | 14,577 | — | 5308 | 6667 | — | 2 | 15 | 0.133 |
| FNNFTVSFWLRVPKVSASHLEQY | 351 | 2824 | 1677 | 2857 | 10,205 | 7 | 15 | 0.467 |
| GDVVAVVDIKEKGKDXWIELK | 15,889 | — | 2294 | 10,000 | — | 1 | 15 | 0.067 |
| GYKVLVLNPSVAAT | 103 | — | 135 | 3695 | — | 12 | 15 | 0.800 |
| IVHATGFKQSSKALQRPVASDFEP | 369 | ND | 763 | ND | 1000 | 7 | 10 | 0.700 |
| LLPLLEKVIGAGKPL | — | — | 1597 | 690 | 180 | 3 | 15 | 0.200 |
| NGQIGNDPNRDIL | — | 100 | — | — | — | 1 | 15 | 0.067 |
| NPVVHFFKNIVTPRTPPPSQ | 24 | ND | 353 | 153 | 199 | 11 | 14 | 0.786 |
| PHHTALRQAILCWGELMTLA | 21 | 575 | 215 | 218 | 576 | 9 | 15 | 0.600 |
| PKYVKQNTLKLAT | 16,384 | 10,000 | 1870 | 42 | — | 7 | 15 | 0.467 |
| PLGFFPDHQLDPAFGANSNNPDWDFNP | 1977 | 311 | 3142 | — | — | 2 | 15 | 0.133 |
| QNILLSNAPLGPQFP | 213 | — | 1105 | — | ND | 6 | 14 | 0.429 |
| QYIKANSKFIGITE | 15,274 | 2971 | 16,268 | 29 | — | 7 | 15 | 0.487 |
| RDTGILDSIGRFFGGDRGAP | 8296 | ND | 3324 | — | 2173 | 2 | 14 | 0.143 |
| VDAQGTLSKIFKLGGRDSRS | — | 769 | 2828 | 6667 | 188 | 6 | 15 | 0.400 |
| WTTCQSIAFPSKTSASIGSL | 556 | — | 336 | 400 | 7.4 | 6 | 14 | 0.429 |
| YKTIAYDEEARR | — | — | — | — | — | 1 | 15 | 0.067 |
| YLDPLIRGLLARPAKLQV | 149 | — | 341 | 476 | 10,208 | 8 | 15 | 0.533 |
| YSGPLKAEIAQRLEDV | — | 1333 | 4454 | 351 | — | 4 | 15 | 0.267 |
| YTLLQAAPALDKLKLTGDEATGANI | 1043 | 3849 | 2315 | 6.3 | — | 6 | 15 | 0.400 |
| Overall hit rate | 10 | 7 | 8 | 9 | 9 | 150 | 425 | 0.353 |

Of the 48 reported restriction events, 31 (65%) were associated with degenerate binding, as defined by 5/15 = 33% of alleles tested.

TABLE 7
Binding Capacity of Cross-Reactive Peptides

Erythropoietin

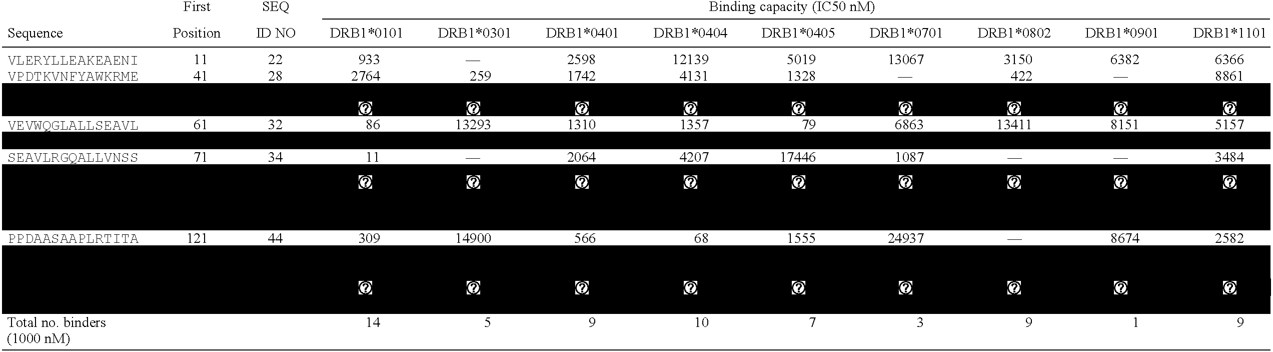

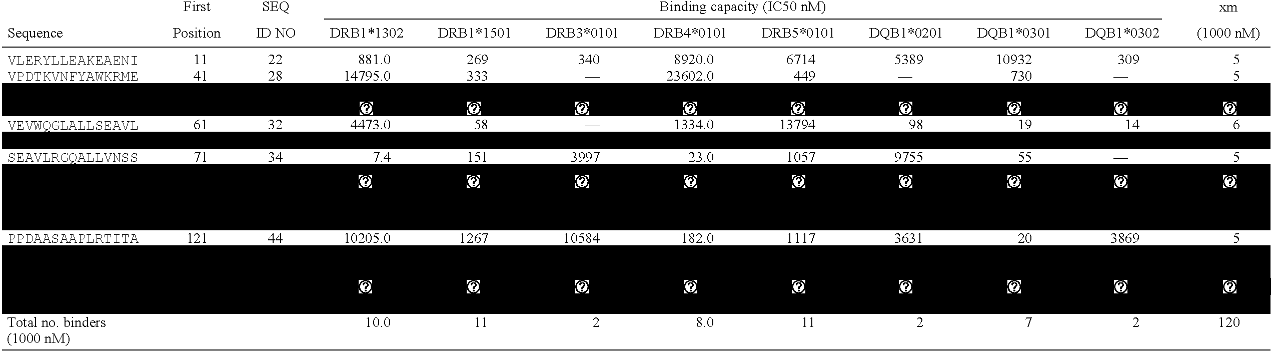

Human Growth Hormone

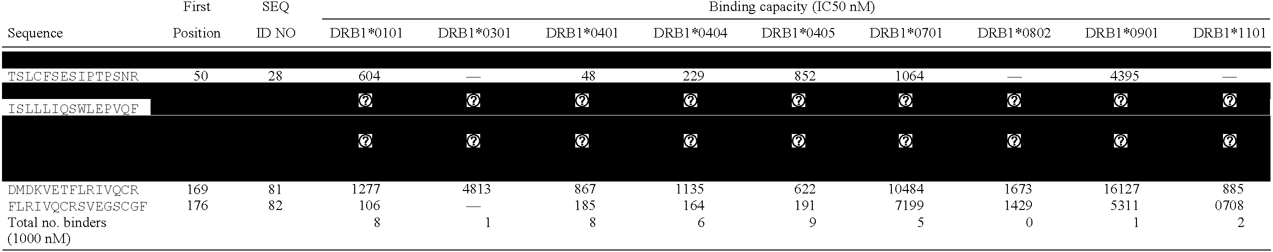

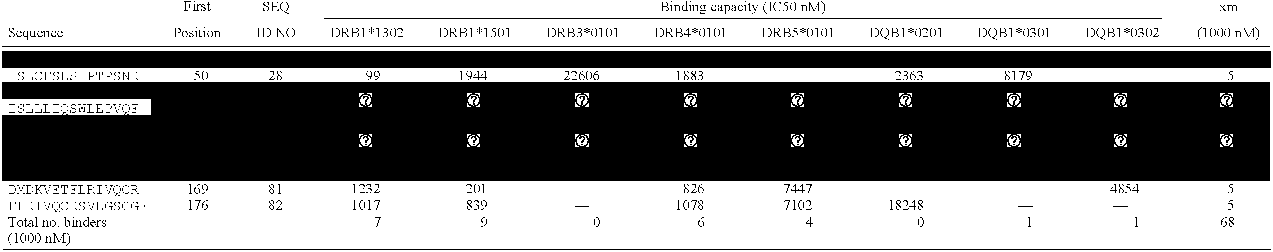

Interferon

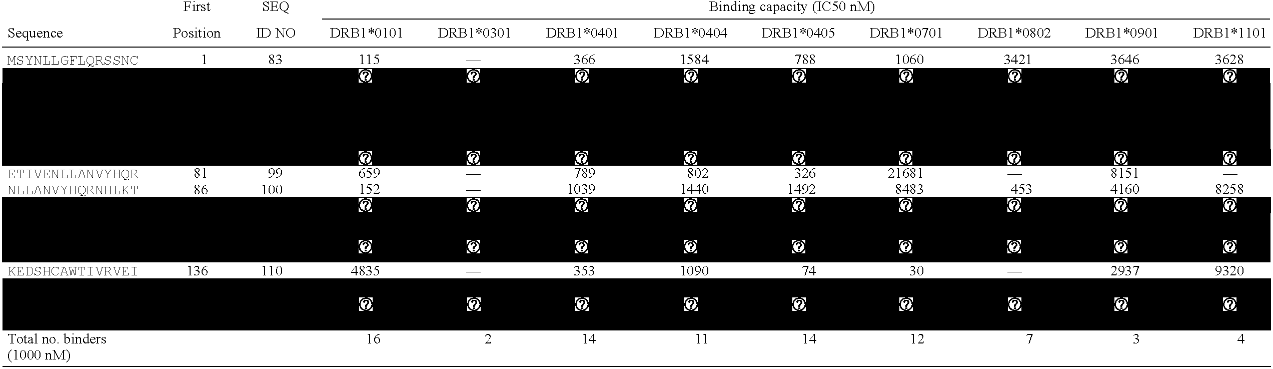

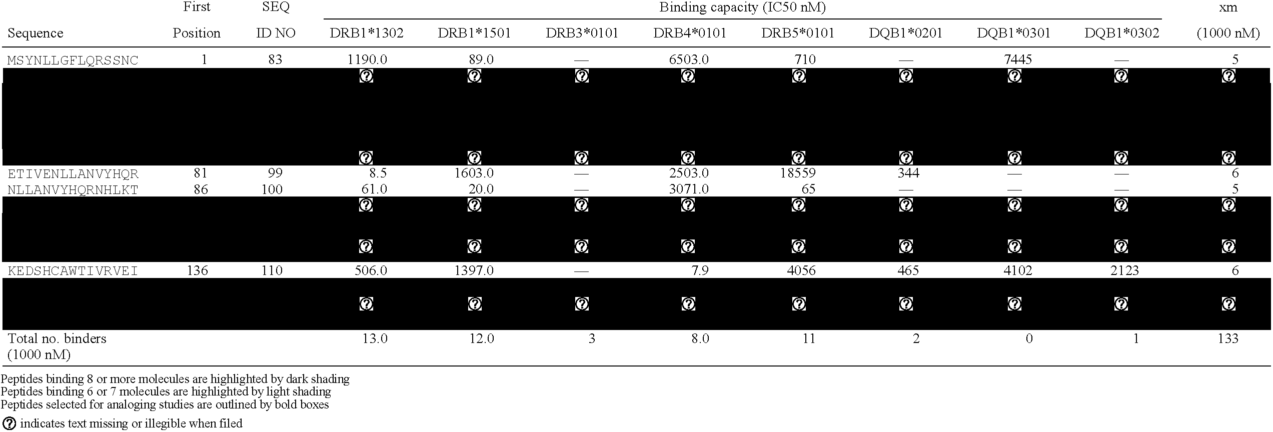

TABLE 8A
MHC Type of Donors Used in the Study

| Donor No | DRB1* | DRB3* | DRB4* | DRB5* | DQB1* |
|---|---|---|---|---|---|
| 1 | 1501, 0701 | neg | 0101 | 01011 | 03032 |
| | | | | | 0602 |
| 2 | 0101, 0405 | neg | 0101 | neg | 0302 |
| | | | | | 05011 |
| 3 | 1301, 0801 | 0101 | neg | neg | 0402 |
| | | | | | 0603 |
| 4 | 0301, 1101 | 0101 | neg | neg | 0201/2 |
| | | 02021 | | | 03011/9 |
| 5 | 0901, 1406 | 0101 | 0101 | neg | 03011/9 |
| | | | | | 03032 |
| 6 | 03011 | 0101 | 0101 | neg | 0201/2 |
| | 0701 | | | | |
| 7 | 0101 (0107) | neg | neg | neg | 05011 |

TABLE 8B
Immunogenicity of Candidate Proteins Containing Degenerate
MHC Binding Regions

| | Donors¹ that | | Percentage |
|---|---|---|---|
| | were tested | Immunogenicity² | of donors |
| | for immuno- | obtained | demonstrating |
| Protein | genicity | in donors | immunogenicity |
| hGH | 1, 3, 4, 5, 7 | None | 0 |
| Erythropoietin | 1, 2, 3, 4, 5, 6, 7 | 2, 3, 4, 5, 6 | 71 |

¹Refer to Table 8A for details of MHC typing
²T cell lines were obtained by in vitro induction of PBMC with peptide pulsed DC, followed by multiple stimulations with peptide pulsed PBMC

TABLE 9
Summary of Antigenic Peptides Derived From EPO

| | | | Binding^{a} | |
|---|---|---|---|---|
| | SEQ | PIC | IC50 | |
| EPO | ID NO | 125 nM | (1000 nM) | Antigenic^{b} |
| WKRMEVGQQAVEVWQ | 30 | + | + | + |
| GLALLSEAVLRGQAL | 33 | + | + | + |
| QPWEPLQLHVDKAVS | 37 | − | − | + |
| LQLHVDKAVSGLRSL | 38 | + | + | + |
| DKAVSGLRSLTTLLR | 39 | + | + | + |
| GLRSLTTLLRALGAQ | 40 | + | + | + |
| TTLLRALGAQKEAIS | 41 | + | + | + |
| RTITADTFRKLFRVY | 46 | − | + | + |
| DTFRKLFRVYSNFLR | 47 | + | + | + |
| | | 6/9 | 8/9 | 9 |
| TOTAL Accuracy | | 13/16 | 8/16 | 16 |
| | | (81%) | (50%) | |

^{a}Only peptides binding ≧6/15 molecules at IC50 ≦1000 nM were considered positive
^{b}Antigenicity was determined by screening for ELISPOT responses against T cell lines generated against whole protein

TABLE 10A
EPO 101 Analog Binding Results

| | SEQ | Binding capacity (IC50 nM) | | | | | | |
|---|---|---|---|---|---|---|---|---|
| Sequence | ID NO | DRB1*0101 | DRB1*0301 | DRB1*0401 | DRB1*0404 | DRB1*0405 | DRB1*0701 | DRB1*0802 |
| GLRSLTTLLRALGAQ | 40 | 8.5 | 2345 | 24 | 9.2 | 30 | 1217 | 14 |
| GARSLTTLLRALGAQ | 152 | 45 | 19468 | 1132 | 97 | 1298 | — | 16 |
| GDRSLTTLLRALGAQ | 153 | 73 | — | 982 | 121 | 8723 | — | 20 |
| GERSLTTLLRALGAQ | 154 | 31 | 25472 | 238 | 93 | 9187 | — | 33 |
| GGRSLTTLLRALGAQ | 155 | 12 | 25341 | 247 | 90 | 8016 | — | 648 |
| GHRSLTTLLRALGAQ | 156 | 72 | 13870 | 301 | 101 | 1252 | — | 48 |
| GKRSLTTLLRALGAQ | 157 | 12 | 11445 | 120 | 26 | 588 | 14346 | 7.8 |
| GNRSLTTLLRALGAQ | 158 | 25 | — | 215 | 48 | 3711 | — | 25 |
| GPRSLTTLLRALGAQ | 159 | 213 | — | 2631 | 689 | 20181 | — | 1012 |
| GQRSLTTLLRALGAQ | 160 | 24 | 27258 | 345 | 106 | 2839 | — | 40 |
| GRRSLTTLLRALGAQ | 161 | 14 | 14843 | 113 | 25 | 1012 | 3323 | 7.5 |
| GSRSLTTLLRALGAQ | 162 | 79 | 26127 | 567 | 130 | 1509 | — | 88 |
| GTRSLTTLLRALGAQ | 163 | 15 | 14195 | 127 | 76 | 1098 | 22800 | 444 |
| GLRSLTTLARALGAQ | 164 | 14 | 234 | 68 | 13 | 455 | 1647 | 7.1 |
| GLRSLTTLDRALGAQ | 165 | 20 | 272 | 73 | 68 | 158 | — | 15 |
| GLRSLTTLERALGAQ | 166 | 147 | 1446 | 64 | 64 | 1502 | — | 21 |
| GLRSLTTLGRALGAQ | 167 | 87 | 2043 | 41 | 8.1 | 221 | 26665 | 9.3 |
| GLRSLTTLHRALGAQ | 168 | 16 | 240 | 32 | 18 | 186 | 6070 | 13 |
| GLRSLTTLKRALGAQ | 169 | 74 | 1098 | 35 | 13 | 649 | — | 8.5 |
| GLRSLTTLNRALGAQ | 170 | 23 | 1438 | 28 | 69 | 1604 | 18125 | 12 |
| GLRSLTTLPRALGAQ | 171 | 14 | 1305 | 145 | 13 | 524 | 27278 | 7.3 |
| GLRSLTTLQRALGAQ | 172 | 83 | 1316 | 162 | 9.3 | 8776 | — | 48 |
| GLRSLTTLRRALGAQ | 173 | 69 | 667 | 300 | 12 | 706 | — | 7.7 |
| GLRSLTTLSRALGAQ | 174 | 89 | 928 | 186 | 13 | 719 | 4209 | 69 |
| GLRSLTTLTRALGAQ | 175 | 79 | 1972 | 394 | 29 | 2026 | 2710 | 30 |
| GLDSLTTLLRALGAQ | 176 | 90 | 15816 | 1597 | 421 | 1817 | — | 68 |
| GLGSLTTLLRALGAQ | 177 | 69 | 6328 | 244 | 98 | 2173 | — | 58 |
| GLRDLTTLLRALGAQ | 178 | 37 | 4464 | 474 | 132 | 194 | — | 15 |
| GLRGLTTLLRALGAQ | 179 | 13 | 1939 | 188 | 13 | 193 | — | 15 |
| GLRRLTTLLRALGAQ | 180 | 15 | 1485 | 1118 | 59 | 1231 | — | 116 |
| GLRSDTTLLRALGAQ | 181 | 44 | 11 | 15 | 110 | 6259 | 27321 | 402 |
| GLRSGTTLLRALGAQ | 182 | 25 | 127 | 237 | 121 | 1511 | 23866 | 70 |
| GLRSRTTLLRALGAQ | 183 | 13 | 115 | 145 | 68 | 1415 | 4156 | 74 |
| GLRSLDTLLRALGAQ | 184 | 21 | 303 | 143 | 14 | 1159 | — | 23 |
| GLRSLGTLLRALGAQ | 185 | 16 | 1977 | 161 | 13 | 154 | 25437 | 14 |
| GLRSLRTLLRALGAQ | 186 | 11 | 243 | 128 | 13 | 436 | 26737 | 12 |
| GLRSLTDLLRALGAQ | 187 | 162 | 2329 | 1061 | 628 | 8794 | — | 75 |
| GLRSLTGLLRALGAQ | 188 | 9.6 | 1697 | 2507 | 143 | 4031 | — | 12 |
| GLRSLTRLLRALGAQ | 189 | 3.6 | 121 | 309 | 143 | 1300 | 20699 | 11 |
| GLRSLTTDLRALGAQ | 190 | 21 | 17 | 107 | 67 | 1480 | — | 75 |
| GLRSLTTGLRALGAQ | 191 | 19 | 26 | 153 | 56 | 3476 | 4877 | 56 |
| GLRSLTTRLRALGAQ | 192 | 2.4 | 1043 | 211 | 70 | 1322 | 4368 | 49 |
| GLRSLTTLLDALGAQ | 193 | 14 | 12983 | 8.5 | 9.3 | 11 | 10347 | 240 |
| GLRSLTTLLGALGAQ | 194 | 1.3 | 14876 | 1.7 | 6.2 | 11 | 11116 | 61 |
| GLRSLTTLLRALGDQ | 195 | 11 | 199 | 147 | 5.8 | 404 | 9328 | 23 |
| GLRSLTTLLRALGGQ | 196 | 9.6 | 259 | 84 | 12 | 246 | 10054 | 20 |
| GLRSLTTLLRALGRQ | 197 | 10 | 240 | 14 | 9.6 | 129 | 11069 | 8.4 |
| Core regions: | | | | | | | | |
| GLRSLTTLLRALGAQ | 40 | Primary | | | | | | |
| GLRSLTTLLRALGAQ | 40 | Secondary core is slightly truncated (by 2 residues at the C-terminus) | | | | | | |
| | | from core present in subsequent peptide that scores very high in PIC and is degenerate. | | | | | | |
| | SEQ | Binding capacity (IC50 nM) | | | | | | |
| Sequence | ID NO | DRB1*0901 | DRB1*1101 | DRB1*1302 | DRB1*1501 | DRB3*0101 | DRB4*0101 | DRB5*0101 |
| GLRSLTTLLRALGAQ | 40 | 1136 | 3.7 | 871 | 11 | 12947 | 283 | 2.7 |
| GARSLTTLLRALGAQ | 152 | 1483 | 3.7 | 4628 | 282 | — | 221 | 71 |
| GDRSLTTLLRALGAQ | 153 | 344 | 2.7 | 6824 | | | 274 | 2.4 |
| GERSLTTLLRALGAQ | 154 | 2035 | 1.1 | 5670 | 214 | — | 465 | 20 |
| GGRSLTTLLRALGAQ | 155 | 1668 | 6.2 | 5504 | 280 | — | 484 | 74 |
| GHRSLTTLLRALGAQ | 156 | 251 | 1.6 | 6434 | | | 308 | 6.1 |
| GKRSLTTLLRALGAQ | 157 | 206 | 0.36 | 851 | | | 61 | 18 |
| GNRSLTTLLRALGAQ | 158 | 326 | 29 | 5253 | | | 281 | 822 |
| GPRSLTTLLRALGAQ | 159 | — | 47 | — | 935 | — | 1486 | 251 |
| GQRSLTTLLRALGAQ | 160 | 404 | 2.1 | 6049 | | | 265 | 27 |
| GRRSLTTLLRALGAQ | 161 | 168 | 3.5 | 1552 | | | 107 | 143 |
| GSRSLTTLLRALGAQ | 162 | 2615 | 4.5 | 5324 | 264 | — | 506 | 65 |
| GTRSLTTLLRALGAQ | 163 | 301 | 1.7 | 929 | | | 137 | 7.4 |
| GLRSLTTLARALGAQ | 164 | 397 | 0.93 | 610 | | | 105 | 4.2 |
| GLRSLTTLDRALGAQ | 165 | 3228 | 3.2 | 5140 | | | 23 | 9.5 |
| GLRSLTTLERALGAQ | 166 | 2500 | 3.1 | 8081 | | | 177 | 26 |
| GLRSLTTLGRALGAQ | 167 | 3693 | 0.79 | 2479 | | | 187 | 5.1 |
| GLRSLTTLHRALGAQ | 168 | 257 | 0.53 | 2983 | | | 174 | 3.8 |
| GLRSLTTLKRALGAQ | 169 | 1923 | 0.58 | 994 | | | 157 | 3.7 |
| GLRSLTTLNRALGAQ | 170 | 337 | 0.97 | 4.2 | | | 167 | 0.89 |
| GLRSLTTLPRALGAQ | 171 | 17410 | 0.62 | 38 | | | 112 | 2.8 |
| GLRSLTTLQRALGAQ | 172 | 2167 | 1.3 | 1077 | 281 | | 189 | 8.7 |
| GLRSLTTLRRALGAQ | 173 | 1550 | 0.79 | 2287 | | | 168 | 1.6 |
| GLRSLTTLSRALGAQ | 174 | 2234 | 1.4 | 440 | | | 180 | 21 |
| GLRSLTTLTRALGAQ | 175 | 1990 | 3.0 | 946 | | | 351 | 13 |
| GLDSLTTLLRALGAQ | 176 | 18314 | 4.8 | 5479 | 523 | — | 1246 | 149 |
| GLGSLTTLLRALGAQ | 177 | 2419 | 3.8 | 4685 | 320 | — | 744 | 638 |
| GLRDLTTLLRALGAQ | 178 | 2529 | 0.99 | 4358 | | | 204 | 5.8 |
| GLRGLTTLLRALGAQ | 179 | 2316 | 3.5 | 4858 | | | 244 | 50 |
| GLRRLTTLLRALGAQ | 180 | 3079 | 5.8 | 5257 | 147 | — | 201 | 10 |
| GLRSDTTLLRALGAQ | 181 | 1758 | 38 | 113 | 703 | 1442 | 284 | 177 |
| GLRSGTTLLRALGAQ | 182 | 264 | 5.0 | 377 | | | 212 | 114 |
| GLRSRTTLLRALGAQ | 183 | 224 | 49 | 437 | | | 243 | 170 |
| GLRSLDTLLRALGAQ | 184 | 12292 | 3.7 | 2499 | | | 421 | 731 |
| GLRSLGTLLRALGAQ | 185 | 2012 | 1.4 | 739 | | | 473 | 13 |
| GLRSLRTLLRALGAQ | 186 | 2332 | 3.7 | 1493 | | | 21 | 82 |
| GLRSLTDLLRALGAQ | 187 | 16387 | 17 | 10939 | 1143 | — | 642 | 1385 |
| GLRSLTGLLRALGAQ | 188 | 2639 | 4.6 | 697 | | | 481 | 51 |
| GLRSLTRLLRALGAQ | 189 | 1279 | 3.1 | 522 | | | 180 | 157 |
| GLRSLTTDLRALGAQ | 190 | 2885 | 5.8 | 49 | | | 89 | 416 |
| GLRSLTTGLRALGAQ | 191 | 13895 | 2.4 | 780 | | | 191 | 13 |
| GLRSLTTRLRALGAQ | 192 | 1884 | 0.79 | 391 | | | 280 | 13 |
| GLRSLTTLLDALGAQ | 193 | 277 | 10 | 953 | | | 154 | 804 |
| GLRSLTTLLGALGAQ | 194 | 1443 | 2.6 | 858 | | | 13 | 294 |
| GLRSLTTLLRALGDQ | 195 | 201 | 1.3 | 642 | | | 980 | 252 |
| GLRSLTTLLRALGGQ | 196 | 316 | 1.8 | 532 | | | 290 | 3.5 |
| GLRSLTTLLRALGRQ | 197 | 1527 | 1.8 | 825 | | | 94 | 6.5 |

| | | |
|---|---|---|
| Core regions: | | |
| GLRSLTTLLRALGAQ | 40 | Primary |
| GLRSLTTLLRALGAQ | 40 | Secondary core is slightly truncated (by 2 residues at the C-terminus) |
| | | from core present in subsequent peptide that scores very high in PIC and is degenerate. |

| | SEQ | Binding capacity (IC50 nM) | | | | | Comp | 10x | Selected |
|---|---|---|---|---|---|---|---|---|---|
| Sequence | ID NO | DQB1*0201 | DQB1*0301 | DQB1*0302 | Bound | Tested | Ratio | red. | candidate |
| GLRSLTTLLRALGAQ | 40 | 1583 | — | — | 10 | 17 | 1.000 | 0 | |
| GARSLTTLLRALGAQ | 152 | 4299 | — | — | 7 | 17 | 0.700 | 7 | ✓ |
| GDRSLTTLLRALGAQ | 153 | | | | 8 | 12 | 0.889 | 5 | |
| GERSLTTLLRALGAQ | 154 | 1581 | — | — | 8 | 17 | 0.600 | 6 | ✓ |
| GGRSLTTLLRALGAQ | 155 | 2553 | 4682 | — | 8 | 17 | 0.600 | 8 | ✓ |
| GHRSLTTLLRALGAQ | 156 | | | | 8 | 12 | 0.889 | 4 | |
| GKRSLTTLLRALGAQ | 157 | | | | 10 | 12 | 1.111 | 2 | |
| GNRSLTTLLRALGAQ | 158 | | | | 8 | 12 | 0.889 | 4 | |
| GPRSLTTLLRALGAQ | 159 | 13455 | — | — | 5 | 17 | 0.600 | 13 | ✓ |
| GQRSLTTLLRALGAQ | 160 | | | | 8 | 12 | 0.889 | 5 | |
| GRRSLTTLLRALGAQ | 161 | | | | 8 | 12 | 0.889 | 2 | |
| GSRSLTTLLRALGAQ | 162 | 1502 | — | — | 8 | 17 | 0.600 | 8 | ✓ |
| GTRSLTTLLRALGAQ | 163 | | | | 9 | 12 | 1.000 | 3 | |
| GLRSLTTLARALGAQ | 164 | | | | 11 | 12 | 1.222 | 1 | |
| GLRSLTTLDRALGAQ | 165 | | | | 9 | 12 | 1.000 | 1 | |
| GLRSLTTLERALGAQ | 166 | | | | 7 | 12 | 0.778 | 4 | |
| GLRSLTTLGRALGAQ | 167 | | | | 8 | 12 | 0.889 | 2 | |
| GLRSLTTLHRALGAQ | 168 | | | | 10 | 12 | 1.111 | 0 | |
| GLRSLTTLKRALGAQ | 169 | | | | 9 | 12 | 1.000 | 2 | |
| GLRSLTTLNRALGAQ | 170 | | | | 9 | 12 | 1.000 | 2 | |
| GLRSLTTLPRALGAQ | 171 | | | | 9 | 12 | 1.000 | 3 | |
| GLRSLTTLQRALGAQ | 172 | | | | 8 | 13 | 0.821 | 3 | |
| GLRSLTTLRRALGAQ | 173 | | | | 9 | 12 | 1.000 | 3 | |
| GLRSLTTLSRALGAQ | 174 | | | | 10 | 12 | 1.111 | 2 | |
| GLRSLTTLTRALGAQ | 175 | | | | 8 | 12 | 0.889 | 2 | |
| GLDSLTTLLRALGAQ | 176 | 519 | 8380 | — | 7 | 17 | 0.700 | 9 | ✓ |
| GLGSLTTLLRALGAQ | 177 | 1112 | — | — | 8 | 17 | 0.600 | 7 | ✓ |
| GLRDLTTLLRALGAQ | 178 | | | | 8 | 12 | 0.889 | 3 | |
| GLRGLTTLLRALGAQ | 179 | | | | 8 | 12 | 0.889 | 2 | |
| GLRRLTTLLRALGAQ | 180 | 536 | — | — | 8 | 17 | 0.600 | 5 | ✓ |
| GLRSDTTLLRALGAQ | 181 | 420 | — | — | 11 | 17 | 1.100 | 7 | ✓ |
| GLRSGTTLLRALGAQ | 182 | | | | 10 | 12 | 1.111 | 4 | |
| GLRSRTTLLRALGAQ | 183 | | | | 10 | 12 | 1.111 | 3 | |
| GLRSLDTLLRALGAQ | 184 | | | | 8 | 12 | 0.889 | 4 | |
| GLRSLGTLLRALGAQ | 185 | | | | 9 | 12 | 1.000 | 1 | |
| GLRSLRTLLRALGAQ | 186 | | | | 9 | 12 | 1.000 | 3 | |
| GLRSLTDLLRALGAQ | 187 | 1225 | 8059 | — | 5 | 17 | 0.600 | 10 | ✓ |
| GLRSLTGLLRALGAQ | 188 | | | | 7 | 12 | 0.778 | 5 | |
| GLRSLTRLLRALGAQ | 189 | | | | 9 | 12 | 1.000 | 5 | |
| GLRSLTTDLRALGAQ | 190 | | | | 9 | 12 | 1.000 | 3 | |
| GLRSLTTGLRALGAQ | 191 | | | | 9 | 12 | 1.000 | 2 | |
| GLRSLTTRLRALGAQ | 192 | | | | 8 | 12 | 0.889 | 1 | |
| GLRSLTTLLDALGAQ | 193 | | | | 10 | 12 | 1.111 | 2 | |
| GLRSLTTLLGALGAQ | 194 | | | | 9 | 12 | 1.000 | 1 | |
| GLRSLTTLLRALGDQ | 195 | | | | 11 | 12 | 1.222 | 2 | |
| GLRSLTTLLRALGGQ | 196 | | | | 11 | 12 | 1.222 | 0 | |
| GLRSLTTLLRALGRQ | 197 | | | | 10 | 12 | 1.111 | 0 | |

| | | |
|---|---|---|
| Core regions: | | |
| GLRSLTTLLRALGAQ | 40 | Primary |
| GLRSLTTLLRALGAQ | 40 | Secondary core is slightly truncated (by 2 residues at the C-terminus) |
| | | from core present in subsequent peptide that scores very high in PIC and is degenerate. |

TABLE 10B
EPO 136 Analog Binding Results

| | SEQ | Binding capacity (IC50 nM) | | | | | | |
|---|---|---|---|---|---|---|---|---|
| Sequence | ID NO | DRB1*0101 | DRB1*0301 | DRB1*0401 | DRB1*0404 | DRB1*0405 | DRB1*0701 | DRB1*0802 |
| DTFRKLFRVYSNFLR | 47 | 12 | 8946 | 70 | 104 | 93 | 16 | 39 |
| DTFRKAFRVYSNFLR | 198 | 68 | 1414 | 25 | 486 | 48 | 737 | 103 |
| DTFRKDFRVYSNFLR | 199 | 148 | 233 | 193 | 1605 | 694 | 12426 | 753 |
| DTFRKEFRVYSNFLR | 200 | 80 | 1649 | 116 | 756 | 110 | 6290 | 119 |
| DTFRKGFRVYSNFLR | 201 | 103 | 3343 | 242 | 151 | 131 | 1833 | 121 |
| DTFRKHFRVYSNFLR | 202 | 80 | 3274 | 32 | 569 | 30 | 239 | 60 |
| DTFRKKFRVYSNFLR | 203 | 46 | 1696 | 16 | 351 | 45 | 1006 | 17 |
| DTFRKNFRVYSNFLR | 204 | 62 | 1929 | 91 | 922 | 75 | 1153 | 30 |
| DTFRKPFRVYSNFLR | 205 | 16 | 3602 | 48 | 641 | 69 | 820 | 116 |
| DTFRKQFRVYSNFLR | 206 | 15 | 1751 | 18 | 270 | 15 | 3004 | 81 |
| DTFRKRFRVYSNFLR | 207 | 49 | 1922 | 15 | 726 | 20 | 1014 | 28 |
| DTFRKSFRVYSNFLR | 208 | 88 | 1004 | 22 | 1002 | 27 | 5102 | 12 |
| DTFRKTFRVYSNFLR | 209 | 100 | 2796 | 35 | 1203 | 16 | 138 | 18 |
| DTARKLFRVYSNFLR | 210 | 15 | 193 | 32 | 165 | 24 | 76 | 13 |
| DTDRKLFRVYSNFLR | 211 | 20 | 103 | 27 | 157 | 8.9 | 143 | 17 |
| DTERKLFRVYSNFLR | 212 | 19 | 1224 | 99 | 134 | 13 | 172 | 77 |
| DTGRKLFRVYSNFLR | 213 | 18 | 1279 | 46 | 175 | 39 | 312 | 53 |
| DTHRKLFRVYSNFLR | 214 | 17 | 2632 | 105 | 170 | 63 | 533 | 43 |
| DTKRKLFRVYSNFLR | 215 | 13 | 6487 | 106 | 127 | 39 | 142 | 16 |
| DTNRKLFRVYSNFLR | 216 | 16 | 2038 | 43 | 202 | 11 | 118 | 54 |
| DTPRKLFRVYSNFLR | 217 | 13 | 1041 | 40 | 128 | 6.9 | 87 | 65 |
| DTQRKLFRVYSNFLR | 218 | 11 | 2407 | 116 | 109 | 30 | 125 | 46 |
| DTRRKLFRVYSNFLR | 219 | 9.7 | 1054 | 42 | 132 | 40 | 147 | 74 |
| DTSRKLFRVYSNFLR | 220 | 33 | 1139 | 17 | 102 | 33 | 128 | 101 |
| DTTRKLFRVYSNFLR | 221 | 37 | 5554 | 17 | 113 | 41 | 94 | 106 |
| DTFRKLDRVYSNFLR | 222 | 23 | 2811 | 190 | 1727 | 288 | 97 | 87 |
| DTFRKLGRVYSNFLR | 223 | 15 | 1377 | 351 | 622 | 106 | 228 | 33 |
| DTFRKLRRVYSNFLR | 224 | 8.5 | 1597 | 44 | 89 | 43 | 102 | 12 |
| DTFRKLFDVYSNFLR | 225 | 98 | 25338 | 295 | 1914 | 325 | 954 | 1528 |
| DTFRKLFGVYSNFLR | 226 | 5.4 | — | — | 202 | 209 | 630 | 142 |
| DTFRKLFRDYSNFLR | 227 | 196 | 120 | — | 2847 | 966 | 17718 | 78 |
| DTFRKLFRGYSNFLR | 228 | 11 | 28797 | — | 2542 | 68 | 1023 | 18 |
| DTFRKLFRRYSNFLR | 229 | 76 | 4118 | — | 2500 | 18 | 857 | 9.7 |
| DTFRKLFRVDSNFLR | 230 | 69 | 52 | — | 97 | 24 | 293 | 38 |
| DTFRKLFRVGSNFLR | 231 | 10 | 1630 | — | 141 | 58 | 100 | 12 |
| DTFRKLFRVRSNFLR | 232 | 7.7 | 1161 | — | 118 | 46 | 85 | 8.2 |
| DTFRKLFRVYDNFLR | 233 | 140 | 15696 | 235 | 1336 | 75 | 6870 | 84 |
| DTFRKLFRVYGNFLR | 234 | 13 | 10530 | 138 | 1840 | 29 | 213 | 17 |
| DTFRKLFRVYRNFLR | 235 | 54 | 192 | 21 | 918 | 11 | 1928 | 13 |
| DTFRKLFRVYSDFLR | 236 | 144 | 10444 | 888 | 6216 | 243 | 988 | 70 |
| DTFRKLFRVYSGFLR | 237 | 86 | 22053 | 233 | 1292 | 145 | 136 | 16 |
| DTFRKLFRVYSRFLR | 238 | 56 | 2317 | 215 | 2558 | 127 | 77 | 8.1 |
| DTFRKLFRVYSNDLR | 239 | 85 | 11477 | 24 | 812 | 487 | 817 | 75 |
| DTFRKLFRVYSNGLR | 240 | 28 | 15865 | 79 | 561 | 63 | 288 | 15 |
| DTFRKLFRVYSNRLR | 241 | 18 | 6576 | 124 | 160 | 136 | 388 | 47 |
| DTFRKLFRVYSNFDR | 242 | 79 | 17292 | 213 | 1083 | 74 | 1411 | 13 |
| DTFRKLFRVYSNFGR | 243 | 90 | 17127 | 50 | 70 | 78 | 3519 | 21 |
| DTFRKLFRVYSNFRR | 244 | 70 | 1321 | 29 | 316 | 20 | 1655 | 10 |
| Core regions: | | | | | | | | |
| DTFRKLFRVYSNFLR | 47 | Primary | | | | | | |
| DTFRKLFRVYSNFLR | 47 | Secondary core does not fit PIC motif, but is compatible with EPITOPE. | | | | | | |
| | SEQ | Binding capacity (IC50 nM) | | | | | | |
| Sequence | ID NO | DRB1*0901 | DRB1*1101 | RB1*1302 | DRB1*1501 | DRB3*0101 | DRB4*0101 | DRB5*0101 |
| DTFRKLFRVYSNFLR | 47 | 307 | 11 | 10 | 1.7 | | 1029 | 26 |
| DTFRKAFRVYSNFLR | 198 | 212 | 5.3 | 123 | | | 242 | 80 |
| DTFRKDFRVYSNFLR | 199 | 1999 | 56 | 299 | 4.0 | 1669 | 11564 | 878 |
| DTFRKEFRVYSNFLR | 200 | 219 | 17 | 134 | | | 1314 | 19 |
| DTFRKGFRVYSNFLR | 201 | 250 | 10 | 153 | | | 1797 | 25 |
| DTFRKHFRVYSNFLR | 202 | 176 | 9.8 | 116 | | | 1189 | 144 |
| DTFRKKFRVYSNFLR | 203 | 233 | 3.7 | 64 | | | 260 | 96 |
| DTFRKNFRVYSNFLR | 204 | 310 | 5.0 | 29 | | | 1088 | 33 |
| DTFRKPFRVYSNFLR | 205 | 188 | 6.2 | 87 | | | 173 | 92 |
| DTFRKQFRVYSNFLR | 206 | 216 | 9.1 | 115 | | | 56 | 4.9 |
| DTFRKRFRVYSNFLR | 207 | 259 | 9.4 | 85 | | | 554 | 77 |
| DTFRKSFRVYSNFLR | 208 | 159 | 3.2 | 144 | | | 452 | 6.2 |
| DTFRKTFRVYSNFLR | 209 | 231 | 15 | 156 | | | 1351 | 33 |
| DTARKLFRVYSNFLR | 210 | 114 | 8.8 | 5.4 | | | 400 | 22 |
| DTDRKLFRVYSNFLR | 211 | 185 | 26 | 7.6 | | | 1131 | 13 |
| DTERKLFRVYSNFLR | 212 | 176 | 171 | 37 | | | 1349 | 31 |
| DTGRKLFRVYSNFLR | 213 | 161 | 26 | 7.5 | | | 1125 | 12 |
| DTHRKLFRVYSNFLR | 214 | 126 | 11 | 8.2 | | | 994 | 65 |
| DTKRKLFRVYSNFLR | 215 | 105 | 19 | 4.9 | | | 738 | 20 |
| DTNRKLFRVYSNFLR | 216 | 157 | 14 | 6.5 | | | 1197 | 20 |
| DTPRKLFRVYSNFLR | 217 | 28 | 15 | 4.5 | | | 418 | 19 |
| DTQRKLFRVYSNFLR | 218 | 114 | 9.2 | 4.8 | | | 522 | 20 |
| DTRRKLFRVYSNFLR | 219 | 58 | 5.4 | 4.4 | | | 405 | 15 |
| DTSRKLFRVYSNFLR | 220 | 191 | 12 | 9.3 | | | 1053 | 39 |
| DTTRKLFRVYSNFLR | 221 | 151 | 7.5 | 13 | | | 1101 | 11 |
| DTFRKLDRVYSNFLR | 222 | 2406 | 15 | 93 | | | 242 | 119 |
| DTFRKLGRVYSNFLR | 223 | 2019 | 1.7 | 35 | | | 1681 | 324 |
| DTFRKLRRVYSNFLR | 224 | 209 | 5.4 | 3.6 | | | 225 | 16 |
| DTFRKLFDVYSNFLR | 225 | 3753 | 118 | 740 | 375 | — | 10442 | 912 |
| DTFRKLFGVYSNFLR | 226 | 261 | 25 | 467 | 96 | — | — | 677 |
| DTFRKLFRDYSNFLR | 227 | 351 | 28 | 21 | 51 | 68 | — | 2692 |
| DTFRKLFRGYSNFLR | 228 | 43 | 1.7 | 23 | 156 | — | — | 57 |
| DTFRKLFRRYSNFLR | 229 | 221 | 4.5 | 136 | 2.0 | — | — | 9.1 |
| DTFRKLFRVDSNFLR | 230 | 216 | 3.7 | 57 | | | — | 61 |
| DTFRKLFRVGSNFLR | 231 | 154 | 2.3 | 57 | | | — | 8.2 |
| DTFRKLFRVRSNFLR | 232 | 141 | 0.91 | 3.1 | | | — | 7.3 |
| DTFRKLFRVYDNFLR | 233 | 2522 | 4.2 | 5797 | 24 | — | 2415 | 79 |
| DTFRKLFRVYGNFLR | 234 | 1237 | 3.0 | 13 | | | 2365 | 9.2 |
| DTFRKLFRVYRNFLR | 235 | 1695 | 2.8 | 103 | | | 629 | 21 |
| DTFRKLFRVYSDFLR | 236 | 328 | 4.2 | 3382 | 25 | 1118 | 308 | 14 |
| DTFRKLFRVYSGFLR | 237 | 316 | 3.5 | 412 | | | 1483 | 12 |
| DTFRKLFRVYSRFLR | 238 | 245 | 0.81 | 100 | | | 1403 | 8.8 |
| DTFRKLFRVYSNDLR | 239 | 1635 | 3.6 | 66 | | | 1395 | 209 |
| DTFRKLFRVYSNGLR | 240 | 299 | 2.2 | 18 | | | 2461 | 64 |
| DTFRKLFRVYSNRLR | 241 | 256 | 3.3 | 5.1 | | | 1757 | 18 |
| DTFRKLFRVYSNFDR | 242 | 278 | 3.6 | 523 | | | 1935 | 35 |
| DTFRKLFRVYSNFGR | 243 | 1629 | 2.8 | 163 | | | 1682 | 24 |
| DTFRKLFRVYSNFRR | 244 | 1337 | 0.64 | 148 | | | 1185 | 12 |

| | | |
|---|---|---|
| Core regions: | | |
| DTFRKLFRVYSNFLR | 47 | Primary |
| DTFRKLFRVYSNFLR | 47 | Secondary core does not fit PIC motif, but is compatible with EPITOPE. |

| | SEQ | Binding capacity (IC50 nM) | | | | | | 10x | Selected |
|---|---|---|---|---|---|---|---|---|---|
| Sequence | ID NO | DQB1*0201 | DQB1*0301 | DQB1*0302 | Bound | Tested | | red. | candidate |
| DTFRKLFRVYSNFLR | 47 | 8188 | 2243 | — | 11 | 17 | 1.000 | 0 | |
| DTFRKAFRVYSNFLR | 198 | | | | 11 | 12 | 1.100 | 2 | |
| DTFRKDFRVYSNFLR | 199 | 4708 | — | — | 9 | 17 | 0.818 | 8 | ✓ |
| DTFRKEFRVYSNFLR | 200 | | | | 9 | 12 | 0.900 | 2 | |
| DTFRKGFRVYSNFLR | 201 | | | | 9 | 12 | 0.900 | 2 | |
| DTFRKHFRVYSNFLR | 202 | | | | 10 | 12 | 1.000 | 2 | |
| DTFRKKFRVYSNFLR | 203 | | | | 10 | 12 | 1.000 | 1 | |
| DTFRKNFRVYSNFLR | 204 | | | | 9 | 12 | 0.900 | 1 | |
| DTFRKPFRVYSNFLR | 205 | | | | 11 | 12 | 1.100 | 1 | |
| DTFRKQFRVYSNFLR | 206 | | | | 10 | 12 | 1.000 | 2 | |
| DTFRKRFRVYSNFLR | 207 | | | | 10 | 12 | 1.000 | 1 | |
| DTFRKSFRVYSNFLR | 208 | | | | 9 | 12 | 0.900 | 2 | |
| DTFRKTFRVYSNFLR | 209 | | | | 9 | 12 | 0.900 | 2 | |
| DTARKLFRVYSNFLR | 210 | | | | 12 | 12 | 1.200 | 0 | |
| DTDRKLFRVYSNFLR | 211 | | | | 11 | 12 | 1.100 | 0 | |
| DTERKLFRVYSNFLR | 212 | | | | 10 | 12 | 1.000 | 2 | |
| DTGRKLFRVYSNFLR | 213 | | | | 10 | 12 | 1.000 | 1 | |
| DTHRKLFRVYSNFLR | 214 | | | | 11 | 12 | 1.100 | 1 | |
| DTKRKLFRVYSNFLR | 215 | | | | 11 | 12 | 1.100 | 0 | |
| DTNRKLFRVYSNFLR | 216 | | | | 10 | 12 | 1.000 | 0 | |
| DTPRKLFRVYSNFLR | 217 | | | | 11 | 12 | 1.100 | 0 | |
| DTQRKLFRVYSNFLR | 218 | | | | 11 | 12 | 1.100 | 0 | |
| DTRRKLFRVYSNFLR | 219 | | | | 11 | 12 | 1.100 | 0 | |
| DTSRKLFRVYSNFLR | 220 | | | | 10 | 12 | 1.000 | 0 | |
| DTTRKLFRVYSNFLR | 221 | | | | 10 | 12 | 1.000 | 0 | |
| DTFRKLDRVYSNFLR | 222 | | | | 9 | 12 | 0.900 | 1 | |
| DTFRKLGRVYSNFLR | 223 | | | | 9 | 12 | 0.900 | 2 | |
| DTFRKLRRVYSNFLR | 224 | | | | 11 | 12 | 1.100 | 0 | |
| DTFRKLFDVYSNFLR | 225 | 6241 | 26700 | — | 8 | 17 | 0.727 | 10 | ✓ |
| DTFRKLFGVYSNFLR | 226 | 560 | 8491 | — | 11 | 17 | 1.000 | 7 | ✓ |
| DTFRKLFRDYSNFLR | 227 | — | — | — | 9 | 17 | 0.818 | 10 | ✓ |
| DTFRKLFRGYSNFLR | 228 | 8214 | 15220 | — | 8 | 17 | 0.727 | 5 | ✓ |
| DTFRKLFRRYSNFLR | 229 | — | — | — | 9 | 17 | 0.818 | 7 | ✓ |
| DTFRKLFRVDSNFLR | 230 | | | | 10 | 12 | 1.000 | 3 | |
| DTFRKLFRVGSNFLR | 231 | | | | 9 | 12 | 0.900 | 2 | |
| DTFRKLFRVRSNFLR | 232 | | | | 9 | 12 | 0.900 | 2 | |
| DTFRKLFRVYDNFLR | 233 | 14286 | 5765 | — | 7 | 17 | 0.636 | 5 | ✓ |
| DTFRKLFRVYGNFLR | 234 | | | | 8 | 12 | 0.800 | 2 | |
| DTFRKLFRVYRNFLR | 235 | | | | 10 | 12 | 1.000 | 1 | |
| DTFRKLFRVYSDFLR | 236 | 328 | 19239 | — | 11 | 17 | 1.000 | 6 | ✓ |
| DTFRKLFRVYSGFLR | 237 | | | | 9 | 12 | 0.900 | 2 | |
| DTFRKLFRVYSRFLR | 238 | | | | 9 | 12 | 0.900 | 1 | |
| DTFRKLFRVYSNDLR | 239 | | | | 9 | 12 | 0.900 | 1 | |
| DTFRKLFRVYSNGLR | 240 | | | | 10 | 12 | 1.000 | 1 | |
| DTFRKLFRVYSNRLR | 241 | | | | 10 | 12 | 1.000 | 1 | |
| DTFRKLFRVYSNFDR | 242 | | | | 8 | 12 | 0.800 | 3 | |
| DTFRKLFRVYSNFGR | 243 | | | | 8 | 12 | 0.800 | 2 | |
| DTFRKLFRVYSNFRR | 244 | | | | 8 | 12 | 0.800 | 2 | |

| | | |
|---|---|---|
| Core regions: | | |
| DTFRKLFRVYSNFLR | 47 | Primary |
| DTFRKLFRVYSNFLR | 47 | Secondary core does not fit PIC motif, but is compatible with EPITOPE. |

TABLE 11A
Binding Analysis of EPO 101 Analogs with Reduced Binding Capacity

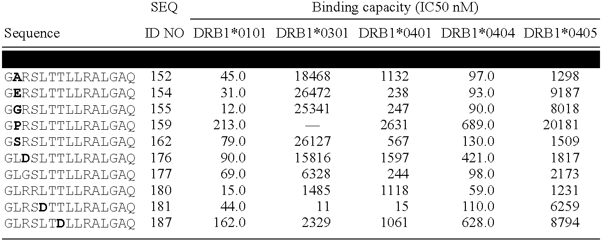

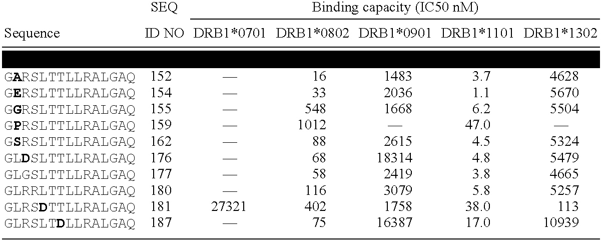

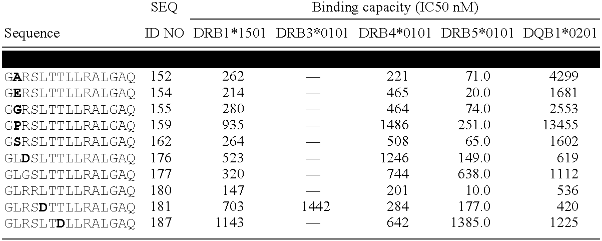

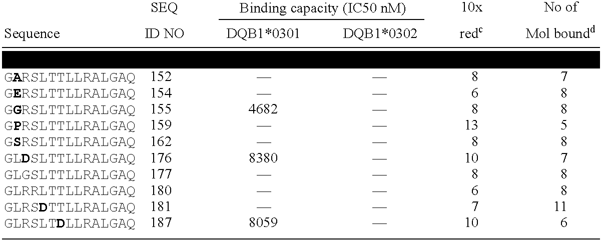

^{a}Wild type peptides indicated by shaded regions
^{b}indicates no binding observed
^{c}indicates the number of MHC molecules to which binding capacity was reduced by ten-fold or more
^{d}number of MHC molecules to which peptide binds at IC₅₀ ≦ 1000 nM

TABLE 11B
Binding Analysis of EPO 136 Analogs with Reduced Binding Capacity

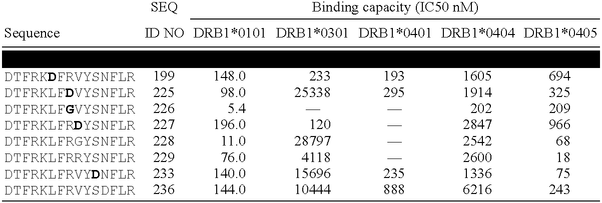

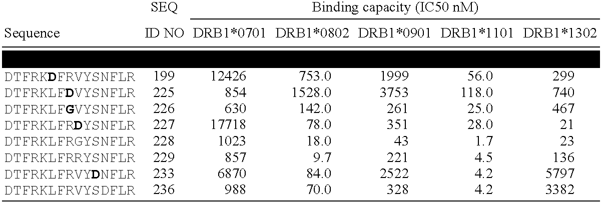

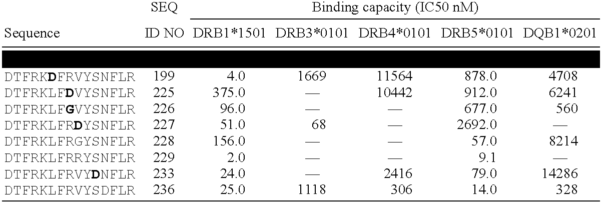

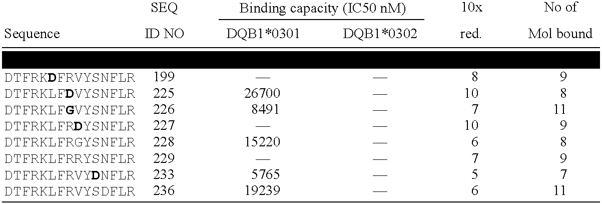

^{a}Wild type peptides indicated by shaded regions

TABLE 12
Antigenicity of EPO Analogs with Reduced Binding Capacity

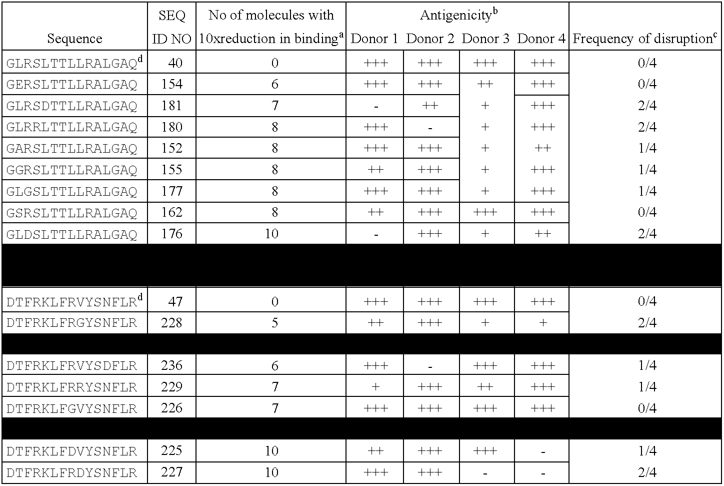

Shaded regions indicate analogs with potential for ImmunoStealth ™ modification
Scale: +++ (70-90%), ++ (40-70%), + (10-40%), − (<10%) of the wildtype peptide response
^{a}indicates the number of MHC molecules in which binding capacity was reduced by ten-fold or more
^{b}Antigenicity was measured in an ELISPOT assay against T cell lines generated against whole protein
^{c}Frequency of donors in which no antigenic response was detected ( − and + were treated as a disruption)
^{d}Wildtype sequences

TABLE 13A
Effect of Modifying Erythropoietin Receptor Contact Residues in
Erythropoietin Analogs

| In Receptor Contact Sites | | | | Not in Receptor Contact Sites | | | |
|---|---|---|---|---|---|---|---|
| | | % | | | | % | |
| | | substitutions | | | | substitutions | |
| | SEQ | with | | | SEQ | with | |
| | ID | decreased | | | ID | decreased | |
| Amino Acid | NO | bioactivity | n | Amino Acid | NO | bioactivity | n |
| GLRSLTTLLRALGAQ | 40 | 83 | 6 | GLRSLTTLLRALGAQ | 40 | 0 | 2 |
| GLRSLTTLLRALGAQ | 40 | 100 | 2 | GLRSLTTLLRALGAQ | 40 | N.D. | — |
| GLRSLTTLLRALGAQ | 40 | 100 | 2 | GLRSLTTLLRALGAQ | 40 | 50 | 2 |
| GLRSLTTLLRALGAQ | 40 | 50 | 2 | GLRSLTTLLRALGAQ | 40 | 0 | 1 |
| DTFRKLFRVYSNFLR | 47 | 0 | 4 | GLRSLTTLLRALGAQ | 40 | 0 | 3 |
| DTFRKLFRVYSNFLR | 47 | 50 | 2 | DTFRKLFRVYSNFLR | 47 | 0 | 1 |
| DTFRKLFRVYSNFLR | 47 | 100 | 2 | DTFRKLFRVYSNFLR | 47 | 0 | 1 |
| DTFRKLFRVYSNFLR | 47 | 100 | 3 | DTFRKLFRVYSNFLR | 47 | N.D. | — |
| | | | | DTFRKLFRVYSNFLR | 47 | 0 | 4 |
| | | | | DTFRKLFRVYSNFLR | 47 | 0 | 2 |
| Total | | | 23 | | | | 16 |

n = number of substitutions tested;
N.D. no data;
Substitution of residues (one-letter code) located in the erythropoietin receptor contact sites are more likely to result in decreased bioactivity (70% of the substitutions in the receptor contact sites).
Few amino acid substitutions outside of the erythropoietin receptor contact areas result in decreased bioactivity (6%).

TABLE 13B
Predicted Impact of Epitope Analogs on Bioactivity

| | | | Predicted |
|---|---|---|---|
| | | Receptor | Bioactivity |
| Substitution | SEQ ID NO | Contact (+/−) | (+/−) |
| GARSLTTLLRALGAQ | 152 | − | + |
| GERSLTTLLRALGAQ | 154 | − | + |
| GGRSLTTLLRALGAQ | 155 | − | + |
| GPRSLTTLLRALGAQ | 159 | − | +/− |
| GSRSLTTLLRALGAQ | 162 | − | + |
| GLDSLTTLLRALGAQ | 176 | + | − |
| GLGSLTTLLRALGAQ | 177 | + | − |
| GLRRLTTLLRALGAQ | 180 | + | − |
| GLRSDTTLLRALGAQ | 181 | − | +/− |
| GLRSLTDLLRALGAQ | 187 | − | + |
| DTFRKDFRVYSNFLR | 199 | − | + |
| DTFRKLFDVYSNFLR | 225 | + | + |
| DTFRKLFGVYSNFLR | 226 | + | + |
| DTFRKLFRDYSNFLR | 227 | − | + |
| DTFRKLFRGYSNFLR | 228 | − | + |
| DTFRKLFRRYSNFLR | 229 | − | + |
| DTFRKLFRVYDNFLR | 233 | − | + |
| DTFRKLFRVYSDFLR | 236 | + | − |

+ for receptor contact indicates that an amino acid is located within an erythropoietin receptor contact site
+ for predicted bioactivity suggests that the bioactivity of this substitution is expected to be equivalent to wild-type erythropoietin

TABLE 14A
Summary of Double versus Single Analogs of the EPO 108 Degenerate Region

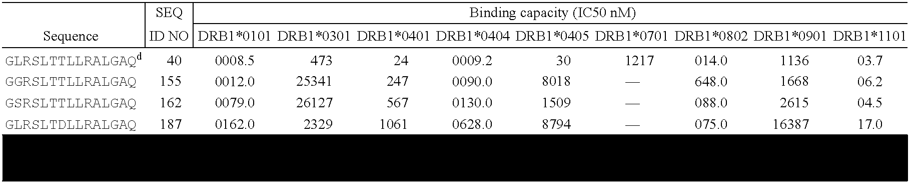

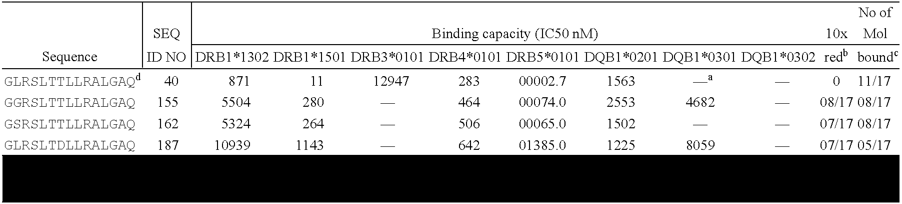

TABLE 14B
Summary of Double versus Single Analogs of the EPO 136 Degenerate Region

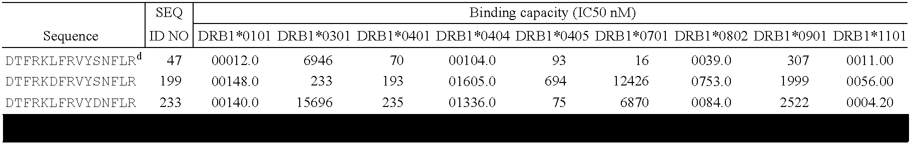

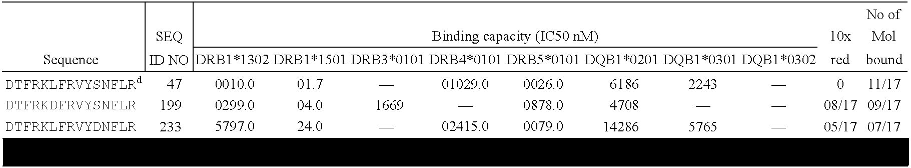

All substitutions are indicated in bold; Double analogs with substantially reduced binding are indicated by shaded regions
^{a}— indicates no binding observed
^{b}indicates the number of MHC molecules to which binding capacity was reduced by tenfold or more
^{c}number of MHC molecules to which peptide binds at IC₅₀ ≦ 1000 nM
^{d}Wildtype epitope sequences

TABLE 14C
Antigenicity of Double Analogs Compared to the Wildtype Peptides

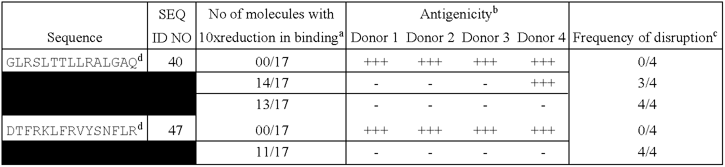

All substitutions are indicated in bold; Double analogs are indicated by shaded regions
^{a}indicates the number of MHC molecules in which binding capacity was reduced by tenfold or more
^{b}Antigenicity was measured in an ELISPOT assay against T cell lines generated against whole protein
^{c}Frequency of donors in which no antigenic response was disrupted ( − and + are treated as a disruption)
^{d}Wildtype epitope sequences

1-26. (canceled)

27. A composition of matter comprising:
a) an isolated or purified modified erythropoietin construct (MEC) selected from the group consisting of: SEQ ID NO: 10, SEQ ID NO: 11, SEQ ID NO: 12, SEQ ID NO: 13, SEQ ID NO: 14, and truncated modified erythropoietin constructs of lengths X to Y, wherein X is an integer selected from 1, 2, 3, 4, 5, or 6 and Y is an integer selected from 188, 189, 190, 191, 192, or 193; or
b) an isolated or purified modified erythropoietin construct according to a), further comprising a heterologous polypeptide sequence; or
c) an isolated MEC of a) or b), and a carrier or diluent; or
d) an isolated, purified, or recombinant nucleic acid encoding a MEC according to a) or b); or
e) an isolated, purified, or recombinant nucleic acid according to d), further comprising regulatory elements, vector elements, or other nucleic acid elements; or
f) a host cell transformed with an isolated nucleic acid according to d) or e); or
g) an isolated, purified or recombinant polynucleotide sequence comprising a sequence encoding a polypeptide sequence selected from the group consisting of SEQ ID NO: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244; or
h) an isolated, purified or recombinant polynucleotide sequence comprising a complementary polynucleotide sequence to a polynucleotide sequence encoding a polypeptide sequence selected from the group consisting of SEQ ID NO: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244; or
i) an isolated, purified or recombinant polynucleotide sequence comprising a polynucleotide sequence having at least about 20% to 99.99% identity to a polynucleotide sequence of g) or h); or
j) a fragment of a polynucleotide sequence according to g) or h); or
k) a peptide, polypeptide, protein, or antibody having reduced immunogenicity as compared to the naturally occurring form of the peptide, polypeptide, or protein while retaining at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of the biological activity of the unmodified or naturally occurring molecule and said reduced immunogenicity is the result of reduced binding to MHC Class II molecules; or
l) a peptide, polypeptide, or protein according to k), wherein the peptide, polypeptide, or protein is a therapeutic peptide, polypeptide, protein or antibody used in the diagnosis or treatment of diseases, conditions, or disorders; or
m) a peptide, polypeptide, protein, or antibody according to c) or k), wherein the peptide, polypeptide, or protein comprises all, or a portion of, IL-1, IL-2, IL-3, IL-4, IL-5, IL-6, IL-7, IL-8, IL-9, IL-10, IL-11, IL-15, IL-16, IL-18, IL-19, IL-23, IL-24, erythropoietin (EPO), insulin, human growth hormone, calcitonin, Factor VIII, G-CSF, M-CSF, GM-CSF platelet derived growth factor (PDGF), MSF, FLT-3 ligand, EGF, fibroblast growth factor (FGF); human insulin alpha, human insulin beta, insulin-like growth factors; vascular endothelial growth factor (VEGF; interferons; leukemia inhibitory factor (LIF); ciliary neurotrophic factor (CNTF); oncostatin M; stem cell factor (SCF); transforming growth factors; chemokines, or antibodies selected from the group consisting of REMICADE® (Infliximab); REOPRO® (Abciximab); SIMULECT® (Basiliximab); ZENAPAX® (Daclizumab); HERCEPTIN® (Trastuzumab); SYNAGIS® (Palivizumab); and XOLAIR® (Omalizumab); or
n) a peptide, polypeptide, protein, or antibody according to m), wherein the protein is EPO and comprises a sequence selected from the group consisting of SEQ ID NOs: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244; or
o) an isolated, purified, or recombinant erythropoietin (EPO) polypeptide containing a substituted peptide segment, wherein said substituted peptide segment is located at positions G101-Q115 (SEQ ID NO: 40) or D136-R150 (SEQ ID NO: 47), and said substituted peptide segment contains at least one amino acid substitution; or
p) an isolated protein of n), comprising an amino acid sequence selected from the group consisting of: SEQ ID NO:10; SEQ ID NO:11; SEQ ID NO:12; SEQ ID NO:13; SEQ ID NO:14; SEQ ID NO:152; SEQ ID NO:154; SEQ ID NO:155; SEQ ID NO:159; SEQ ID NO:162; SEQ ID NO:181; SEQ ID NO:187; SEQ ID NO:199; SEQ ID NO:225; SEQ ID NO:226; SEQ ID NO:227; SEQ ID NO:228; SEQ ID NO:229; and SEQ ID NO:233; SEQ ID NO:245; SEQ ID NO:246; and SEQ ID NO:247; or
q) a peptide, polypeptide, protein, or antibody according to k), l), m), n), o), or p), and a carrier or pharmaceutically acceptable excipient; or
r) an isolated, purified, or recombinant polynucleotide encoding a peptide, polypeptide, protein and/or antibody according to k), l), m), n), o), or p); or
s) an isolated, purified, or recombinant polynucleotide according to r), further comprising regulatory elements selected from promoters, enhancers, termination sequences, and combinations thereof; or
t) a vector comprising an isolated, purified, or recombinant polynucleotide according to r) or s); or
u) a host cell comprising an isolated, purified, or recombinant polynucleotide according to r), s), or t); or
v) an isolated, purified, or recombinant EPO polypeptide according to o), wherein said EPO polypeptide contains substituted peptide segments at positions G101-Q115 (SEQ ID NO: 40) and D136-R150 (SEQ ID NO: 47); or
w) an isolated, purified, or recombinant EPO polypeptide of o) or v), wherein said peptide segments are selected from those peptides set forth in Tables 10A, 10B, 11A, 11B, 12, 13A, 13B, 14A, 14B, or 14C; or
x) an isolated, purified, or recombinant EPO polypeptide of claims o) or v), wherein said peptide segment is selected from the group consisting of: SEQ ID NO:152; SEQ ID NO:154; SEQ ID NO:155; SEQ ID NO:159; SEQ ID NO:162; SEQ ID NO:181; SEQ ID NO:187; SEQ ID NO:199; SEQ ID NO:225; SEQ ID NO:226; SEQ ID NO:227; SEQ ID NO:228; SEQ ID NO:229; and SEQ ID NO:233; SEQ ID NO:245; SEQ ID NO:246; and SEQ ID NO:247.

28. A method of antagonizing an EPO receptor or a method of treating diseases or conditions associated with over-activation of the EPO receptor, comprising administering a composition comprising an EPO variant in amounts sufficient to: 1) block the binding of naturally occurring EPO to its receptor; or 2) reduce the activation levels of the EPO receptor, wherein the EPO variant comprises an amino acid sequence selected from the group consisting of SEQ ID NO: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, and 244, and wherein the EPO variant has the ability to bind to the EPO receptor and fails to activate the EPO receptor.

29. A method of producing a recombinant peptide, polypeptide, protein or antibody comprising the culturing of a host cell according to claim 27 under conditions that allow for the expression of a recombinant peptide, polypeptide, protein or antibody.

30. The method according to claim 29, further comprising the isolation of the recombinant peptide, polypeptide, protein and/or antibody from the host cell or culture system.

31. A method for reducing a helper T lymphocyte (HTL) response against a candidate protein comprising: a) selecting a protein; b) analyzing the amino acid sequence of the protein for potential HTL epitopes; and c) modifying the amino acid sequence of the protein by removing the potential HTL epitope and thereby generating an analog protein.

32. The method according to claim 31, further comprising conducting in vitro antigenicity analysis of said candidate protein using helper T-cells.

33. An isolated, purified, or recombinant polynucleotide comprising a nucleic acid sequence encoding a polypeptide comprising an amino acid sequence selected from the group consisting of SEQ ID NO: 10, 11, 12, 13, 14, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 218, 219, 220, 221, 222, 223, 224, 225, 226, 227, 228, 229, 230, 231, 232, 233, 234, 235, 236, 238, 239, 240, 241, 242, 243, 244, 245, 246, and 247.
